(19) United States
(12) Patent Application Publication
(10) Pub. No.: US 2025/0281746 A1
(43) Pub. Date: Sep. 11, 2025

(54) ELECTRICAL TECHNIQUES FOR PREDICTIVE METHODS AND RELATED TREATMENTS OF A COCHLEA

(71) Applicant: THE UNIVERSITY OF MELBOURNE, Parkville, VIC (AU)

(72) Inventors: Daniel SMYTH, Macquarie University (AU); Robert Daniel GAY, Macquarie University (AU); Ya Lang ENKE, Macquarie University (AU); Stephen O'LEARY, Parkville (AU); Tayla RAZMOVSKI, Parkville (AU); Christofer William BESTER, Parkville (AU); Aaron COLLINS, Parkville (AU)

(21) Appl. No.: 18/859,802

(22) PCT Filed: Apr. 25, 2023
(86) PCT No.: PCT/IB2023/054243

§ 371 Date: Oct. 24, 2024

Related U.S. Application Data
(60) Provisional application No. 63/334,644, filed on Apr. 25, 2022.
(60) Provisional application No. 63/337,924, filed on May 3, 2022.
(60) Provisional application No. 63/455,470, filed on Mar. 29, 2023.

(51) Int. Cl. A61N 1/36; A61B 5/00; A61B 5/0538; A61N 1/05

(52) U.S. Cl. CPC A61N 1/36038; A61B 5/0538; A61B 5/4023; A61B 5/4836; A61N 1/0541

(57) ABSTRACT

A method, comprising obtaining data based on impedance values within a cochlea of a human at a first temporal location and at a second temporal location after the first temporal location, evaluating the obtained data and implementing a treatment based on the evaluation. In an embodiment, the treatment implemented is a steroid treatment.

### CROSS-REFERENCE TO RELATED APPLICATIONS

This application claims priority to U.S. Provisional Application No. 63/334,644, entitled ELECTRICAL TECHNIQUES FOR PREDICTIVE METHODS AND RELATED TREATMENTS OF A COCHLEA, filed on Apr. 25, 2022, naming Stephen O'LEARY as an inventor. This application also claims priority to U.S. Provisional Application No. 63/337,924, entitled ELECTRICAL TECHNIQUES FOR PREDICTIVE METHODS AND RELATED TREATMENTS OF A COCHLEA, filed on May 3, 2022, naming Stephen O'LEARY as an inventor. This application also claims priority to U.S. Provisional Application No. 63/455,470, entitled ELECTRICAL TECHNIQUES FOR PREDICTIVE METHODS AND RELATED TREATMENTS OF A COCHLEA, filed on Mar. 29, 2023, naming Daniel SMYTH as an inventor. The entire contents of each application being incorporated herein by reference in their entirety.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In accordance with an exemplary embodiment, there is a method, comprising, obtaining data based on impedance values within a cochlea of a human at a first temporal location and at a second temporal location after the first temporal location, evaluating the obtained data, and implementing a treatment based on the evaluation.

In an embodiment, there is a method including obtaining first data based on first impedance values obtained with a cochlear implant electrode array located in a cochlea of a human, wherein the impedance values are values for impedances existing within the cochlea within 6 hours of the electrode array first entering the cochlea, obtaining second data based on second impedance values obtained with the cochlear implant electrode array still located in the cochlea of the human, wherein the second impedance values are for impedances existing within the cochlea after at least 12 hours of the electrode array first entering the cochlea, and implementing a treatment and/or increasing an aggressiveness of a treatment only if the second impedance values are greater than the first impedance values.

In an embodiment, there is a method including detecting a near-term physiological change resulting from implantation of a cochlear implant electrode array in a cochlea and/or detecting a near-term latent variable change that is impacted by a physiological change resulting from the implantation of the cochlear implant electrode array using the cochlear implanted electrode array, evaluating a percentage change of the detected near-term physiological change and/or the detected near-term latent variable change and controlling a component of the cochlear implant and/or another implanted component implanted in the human based on the evaluation.

An embodiment includes a non-transitory computer readable medium having recorded thereon, a computer program for executing at least a portion of a method, the computer program including code for statistically analyzing first data based on four-point impedance data obtained using an implanted cochlear implant electrode array in a cochlea during different temporal periods and code for outputting an indication and/or warning.

In an embodiment, there is a method including obtaining a first set of intra-cochlea measurements, wherein the first set of intra-cochlea measurements represent four-point impedance measurements taken by a cochlear implant on the day of implantation, obtaining a second set of intra-cochlea measurements, wherein the second set of intra-cochlea measurements represent four-point impedance measurements taken by the cochlear implant on the day following implantation, comparing data based on the first set to data based on the second set and developing a prognosis based on the comparison and/or developing a performance evaluation based on the comparison.

In an embodiment, there is a method including obtaining a first set of intra-cochlea measurements, wherein the first set of intra-cochlea measurements represent four-point impedance measurements taken by a cochlear implant on the day of implantation, obtaining a second set of intra-cochlea measurements, wherein the second set of intra-cochlea measurements represent four-point impedance measurements taken by the cochlear implant on the day following implantation and determining a fitting profile for the cochlear implant from a comparison of the first set of intra-cochlea measurements with the second set of intra-cochlea measurements.

In an embodiment, there is a system comprising an implantable portion of a cochlear implant electrode array including back telemetry circuity and a plurality of electrodes in signal communication directly or indirectly with the back telemetry circuitry and an external device configured to be in signal communication with the implantable portion and configured to receive data from the implantable portion via the back telemetry circuity, wherein the implantable component is configured to implement four point impedance measurements and provide the results of the measurements to the external device via the back telemetry circuity, and the system is configured to evaluate the results and provide an indication that there is an impedance increase relative to a prior measurement that is predictive of a future deleterious development within a cochlea in which the electrode array is implanted.

### BRIEF DESCRIPTION OF THE DRAWINGS

Embodiments are described below with reference to the attached drawings, in which:

FIG. 1A is a perspective view of an exemplary hearing prosthesis in which at least some of the teachings detailed herein are applicable;

FIG. 1B depicts a side view of the cochlear implant 100 outside of the recipient;

FIG. 1C depicts a vestibular implant;

FIGS. 2A and 2B are side views of an embodiment of an insertion guide for implanting a cochlear implant electrode assembly such as the electrode assembly illustrated in FIG. 1;

FIGS. 3A and 3B are side and perspective views of an electrode assembly extended out of an embodiment of an insertion sheath of the insertion guide illustrated in FIG. 2;

FIGS. 4A-4E are simplified side views depicting the position and orientation of a cochlear implant electrode assembly insertion guide tube relative to the cochlea at each of a series of successive moments during an exemplary implantation of the electrode assembly into the cochlea;

FIGS. 5-9 are exemplary system components of an exemplary embodiment;

FIG. 10 depicts an exemplary four point impedance measurement;

FIG. 11 presents exemplary data according to some embodiments;

FIGS. 12-14 and 20-25 present exemplary algorithms for exemplary methods; and

FIGS. 15-19 present exemplary data according to some embodiments.

### DETAILED DESCRIPTION

Merely for ease of description, the techniques presented herein are described herein with reference by way of background to an illustrative medical device, namely a cochlear implant. However, it is to be appreciated that the techniques presented herein may also be used with a variety of other medical devices that, while providing a wide range of therapeutic benefits to recipients, patients, or other users, may benefit from setting changes based on the location of the medical device. For example, the techniques presented herein may be used to determine the viability of various types of prostheses, such as, for example, a vestibular implant, with respect to a particular human being. Any reference to one of the above-noted sensory prostheses corresponds to an alternate disclosure using one of the other above-noted sensory prostheses unless otherwise noted, providing that the art enables such.

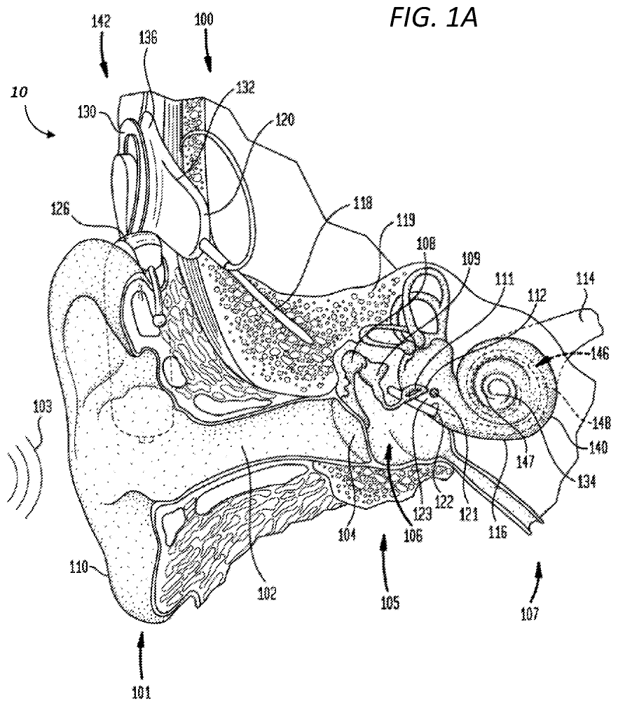

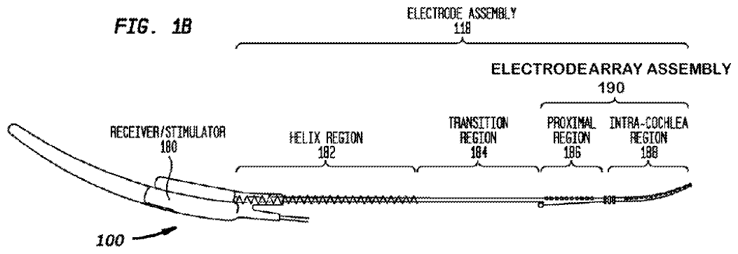

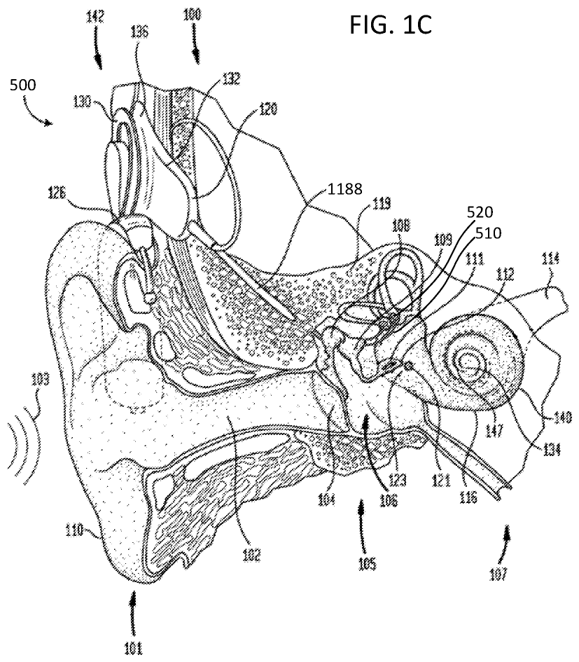

FIG. 1A is a perspective view of a cochlear implant, referred to as cochlear implant 100, implanted in a recipient, to which some embodiments detailed herein and/or variations thereof are applicable. The cochlear implant 100 is part of a system 10 that can include external components in some embodiments, as will be detailed below. It is noted that the teachings detailed herein are also applicable to so-called multi-mode devices. In an exemplary embodiment, these multi-mode devices apply both electrical stimulation and acoustic stimulation to the recipient. In an exemplary embodiment, these multi-mode devices evoke a hearing percept via electrical hearing and bone conduction hearing. Accordingly, any disclosure herein with regard to one of these types of hearing prostheses corresponds to a disclosure of another of these types of hearing prostheses or any medical device for that matter, unless otherwise specified, or unless the disclosure thereof is incompatible with a given device based on the current state of technology.

In view of the above, it is to be understood that at least some embodiments detailed herein and/or variations thereof are directed towards a body-worn sensory supplement medical device (e.g., the hearing prosthesis of FIG. 1A, which supplements the hearing sense, even in instances when there are no natural hearing capabilities, for example, due to degeneration of previous natural hearing capability or to the lack of any natural hearing capability, for example, from birth).

The recipient has an outer ear 101, a middle ear 105, and an inner ear 107. Components of outer ear 101, middle ear 105, and inner ear 107 are described below, followed by a description of cochlear implant 100.

In a fully functional ear, outer ear 101 comprises an auricle 110 and an ear canal 102. An acoustic pressure or sound wave 103 is collected by auricle 110 and channeled into and through ear canal 102. Disposed across the distal end of ear channel 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109, and the stapes 111. Bones 108, 109, and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound.

As shown, cochlear implant 100 comprises one or more components which are temporarily or permanently implanted in the recipient. Cochlear implant 100 is shown in FIG. 1 with an external device 142, that is part of system 10 (along with cochlear implant 100), which, as described below, is configured to provide power to the cochlear implant, where the implanted cochlear implant includes a battery that is rechargeable via the transcutaneous link.

In the illustrative arrangement of FIG. 1A, external device 142 can comprise a power source (not shown) disposed in a Behind-The-Ear (BTE) unit 126. External device 142 also includes components of a transcutaneous energy transfer link, referred to as an external energy transfer assembly. The transcutaneous energy transfer link is used to transfer power and/or data to cochlear implant 100. Various types of energy transfer, such as infrared (IR), electromagnetic, capacitive and inductive transfer, may be used to transfer the power and/or data from external device 142 to cochlear implant 100. In the illustrative embodiments of FIG. 1, the external energy transfer assembly comprises an external coil 130 that forms part of an inductive radio frequency (RF) communication link. External coil 130 is typically a wire antenna coil comprised of multiple turns of electrically insulated single-strand or multi-strand platinum or gold wire. External device 142 also includes a magnet (not shown) positioned within the turns of wire of external coil 130. It should be appreciated that the external device shown in FIG. 1 is merely illustrative, and other external devices may be used with embodiments of the present invention.

Cochlear implant 100 comprises an internal energy transfer assembly 132 which can be positioned in a recess of the temporal bone adjacent auricle 110 of the recipient. As detailed below, internal energy transfer assembly 132 is a component of the transcutaneous energy transfer link and receives power and/or data from external device 142. In the illustrative embodiment, the energy transfer link comprises an inductive RF link, and internal energy transfer assembly 132 comprises a primary internal coil 136. Internal coil 136 is typically a wire antenna coil comprised of multiple turns of electrically insulated single-strand or multi-strand platinum or gold wire.

Cochlear implant 100 further comprises a main implantable component 120 and an elongate electrode assembly 118. In some embodiments, internal energy transfer assembly 132 and main implantable component 120 are hermetically sealed within a biocompatible housing. In some embodiments, main implantable component 120 includes an implantable microphone assembly (not shown) and a sound processing unit (not shown) to convert the sound signals received by the implantable microphone in internal energy transfer assembly 132 to data signals. That said, in some alternative embodiments, the implantable microphone assembly can be located in a separate implantable component (e.g., that has its own housing assembly, etc.) that is in signal communication with the main implantable component 120 (e.g., via leads or the like between the separate implantable component and the main implantable component 120). In at least some embodiments, the teachings detailed herein and/or variations thereof can be utilized with any type of implantable microphone arrangement.

Main implantable component 120 further includes a stimulator unit (also not shown) which generates electrical stimulation signals based on the data signals. The electrical stimulation signals are delivered to the recipient via elongate electrode assembly 118.

Elongate electrode assembly 118 has a proximal end connected to main implantable component 120, and a distal end implanted in cochlea 140. Electrode assembly 118 extends from main implantable component 120 to cochlea 140 through mastoid bone 119. In some embodiments, electrode assembly 118 may be implanted at least in basal region 116, and sometimes further. For example, electrode assembly 118 may extend towards apical end of cochlea 140, referred to as cochlea apex 134. In certain circumstances, electrode assembly 118 may be inserted into cochlea 140 via a cochleostomy 122. In other circumstances, a cochleostomy may be formed through round window 121, oval window 112, the promontory 123 or through an apical turn 147 of cochlea 140.

Electrode assembly 118 comprises a longitudinally aligned and distally extending array 146 of electrodes 148, disposed along a length thereof. As noted, a stimulator unit generates stimulation signals which are applied by electrodes 148 to cochlea 140, thereby stimulating auditory nerve 114.

FIG. 1B is a side view of a cochlear implant 100 without the other components of system 10 (e.g., the external components). Cochlear implant 100 comprises a receiver/stimulator 180 and an electrode assembly or lead 118. Electrode assembly 118 includes a helix region 182, a transition region 184, a proximal region 186, and an intra-cochlear region 188. Proximal region 186 and intra-cochlear region 188 form an electrode array assembly 190. In an exemplary embodiment, proximal region 186 is located in the middle-ear cavity of the recipient after implantation of the intra-cochlear region 188 into the cochlea. Thus, proximal region 186 corresponds to a middle-ear cavity sub-section of the electrode array assembly 190. Electrode array assembly 190, and in particular, intra-cochlear region 188 of electrode array assembly 190, supports a plurality of electrode contacts 148. These electrode contacts 148 are each connected to a respective conductive pathway, such as wires, PCB traces, etc. (not shown) which are connected through lead 118 to receiver/stimulator 180, through which respective stimulating electrical signals for each electrode contact 148 travel.

Electrode array 146 may be inserted into cochlea 140 with the use of an insertion guide. It is noted that while the embodiments detailed herein are described in terms of utilizing an insertion guide or other type of tool to guide the array into the cochlea, in some alternate insertion embodiments, a tool is not utilized. Instead, the surgeon utilizes his or her fingertips or the like to insert the electrode array into the cochlea. That said, in some embodiments, alternate types of tools can be utilized other than and/or in addition to insertion guides. By way of example only and not by way of limitation, surgical tweezers like can be utilized. Any device, system, and/or method of inserting the electrode array into the cochlea can be utilized according to at least some exemplary embodiments.

FIG. 1C depicts an exemplary vestibular implant 500 according to one example. Some specific features are described utilizing the above-noted cochlear implant of FIG. 1A in the context of a vestibular implant. In this regard, some features of a cochlear implant are utilized with vestibular implants. In the interest of textual and pictorial economy, various elements of the vestibular implant that generally correspond to the elements of the cochlear implant above are referenced utilizing the same numerals. Still, it is noted that some features of the vestibular implant 500 will be different from that of the cochlear implant above. By way of example only and not by way of limitation, there may not be a microphone on the behind-the-ear device 126. Alternatively, sensors that have utilitarian value in the vestibular implant can be contained in the BTE device 126. By way of example only and not by way of limitation, motion sensors can be located in BTE device 126. There also may not be a sound processor in the BTE device. Conversely, other types of processors, such as those that process data obtained from the sensors, will be present in the BTE device 126. Power sources, such as a battery, will also be included in the BTE device 126. Consistent with the BTE device of the cochlear implant of FIG. 1, a transmitter/transceiver will be located in the BTE device or otherwise in signal communication therewith.

The implantable component includes a receiver stimulator in a manner concomitant with the above cochlear implant. Here, vestibular stimulator comprises a main implantable component 120 and an elongate electrode assembly 1188 (where the elongate electrode assembly 1188 has some different features from the elongate electrode assembly 118 of the cochlear implant, some of which will be described shortly). In some embodiments, internal energy transfer assembly 132 and main implantable component 120 are hermetically sealed within a biocompatible housing. In some embodiments, main implantable component 120 includes a processing unit (not shown) to convert data obtained by sensors, which could be on board sensors implanted in the recipient, into data signals.

Main implantable component 120 further includes a stimulator unit (also not shown) which generates electrical stimulation signals based on the data signals. The electrical stimulation signals are delivered to the recipient via elongate electrode assembly 1188.

It is briefly noted that while the embodiment shown in FIG. 1C represents a partially implantable vestibular implant, embodiments can include a totally implantable vestibular implant, such as, where, for example, the motion sensors are located in the implantable portion, in a manner analogous to a cochlear implant.

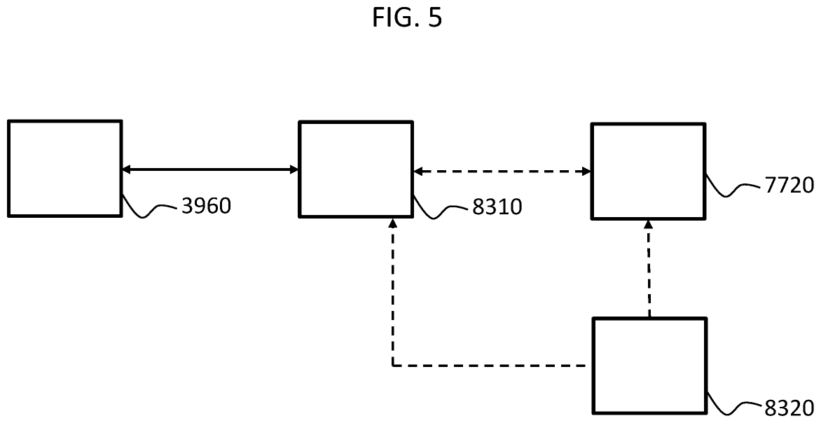

Elongate electrode assembly 1188 has a proximal end connected to main implantable component 120, and extends through a hole in the mastoid 119, in a manner analogous to the elongate electrode assembly 118 of the cochlear implant, and includes a distal end that extends to the inner ear. In some embodiments, the distal portion of the electrode assembly 1188 includes a plurality of leads 510 that branch out away from the main body of the electrode assembly 118 to electrodes 520. Electrodes 520 can be placed at the base of the semicircular ducts as shown in FIG. 5. In an exemplary embodiment, one or more of these electrodes are placed in the vicinity of the vestibular nerve branches innervating the semicircular canals. In some embodiments, the electrodes are located external to the inner ear, while in other embodiments, the electrodes are inserted into the inner ear. Note also while this embodiment does not include an electrode array located in the cochlea, in other embodiments, one or more electrodes are located in the cochlea in a manner analogous to that of a cochlear implant.

Embodiments can include sensory prostheses that use some form of beamforming to improve, for example, hearing performance in some situations. Some embodiments utilize an adaptive beamforming. This can be used to enhance the signal coming from specific directions (such as speech from the front), and/or suppress the sound coming from other directions (such as noise from the rear or sides), which is often unwanted or otherwise can obscure the desired source/noise (such as from the front, which can be a person speaking to the recipient of the prostheses). In some embodiments, this is achieved by analyzing the signal as received from the microphones, for devices that have multiple microphones.

The teachings detailed herein are directed towards identifying phenomenon inside a cochlea (or inside a semi-circular canal/vestibula—the teachings herein can be applicable to an electrode array or an electrode that has been implanted in a semi-circular canal in some scenarios—any disclosure of a cochlea, a cochlear implant, or a cochlear implant electrode array corresponds to an alternate disclosure of a semi-circular canal/vestibula, a vestibular implant or a vestibular implant electrode/electrode array—these are not the same—we are simply using this manner to describe the features in the interests of textual economy). Some embodiments can include utilizing imaging (e.g., CT scan, X-ray, etc.), which require the patient to be exposed to radiation during the process of obtaining medical images, as well as the need for medical equipment in the operating room to provide and otherwise obtain the imaging, as well as a subsequent analysis by an expert to assess the correct insertion of the electrode holder. Some embodiments of the teachings detailed herein utilize such, while other embodiments specifically do not utilize such, but instead utilize other methods to evaluate or otherwise obtain information indicative of a given electrode array insertion scenario. Some embodiments include the action of measuring neuronal activation after stimulation. This exemplary embodiment can require subjective expert analysis and/or can also be dependent on having a good/acceptable neural response. However, in some instances, such is not always obtainable. Again, as with the aforementioned imaging, some embodiments herein utilize such while other embodiments specifically do not utilize such methods. In at least some exemplary embodiments, methods of determining an insertion scenario can utilize voltage measurements in the cochlea. In an exemplary embodiment of such embodiments, the interpretation of the obtained voltage measurements still requires subjective analysis by an expert. In addition, these measurements can be rendered more difficult to interpret than otherwise might be the case by the presence of so-called air bubbles, open electrodes, shorted electrodes, and/or electrode extrusion. Some embodiments of the teachings detailed herein utilize the aforementioned voltage measurements coupled with expert analysis, while in other embodiments some of the teachings detailed herein specifically avoid utilization of expert analysis to obtain or otherwise analyze and electrode array insertion scenario.

Some embodiments include obtaining voltage measurements from inside and/or outside the cochlea and analyzing them in, by way of example only and not by way of limitation, an automated manner, by comparing the voltage measurements to statistical data.

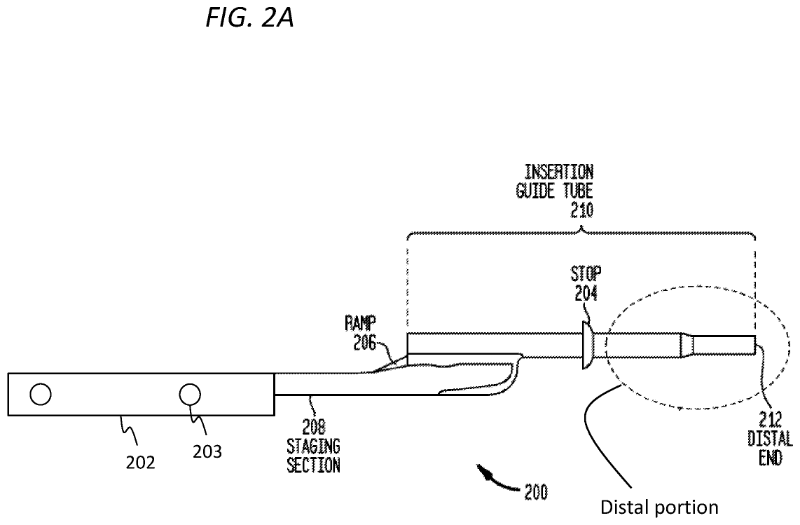

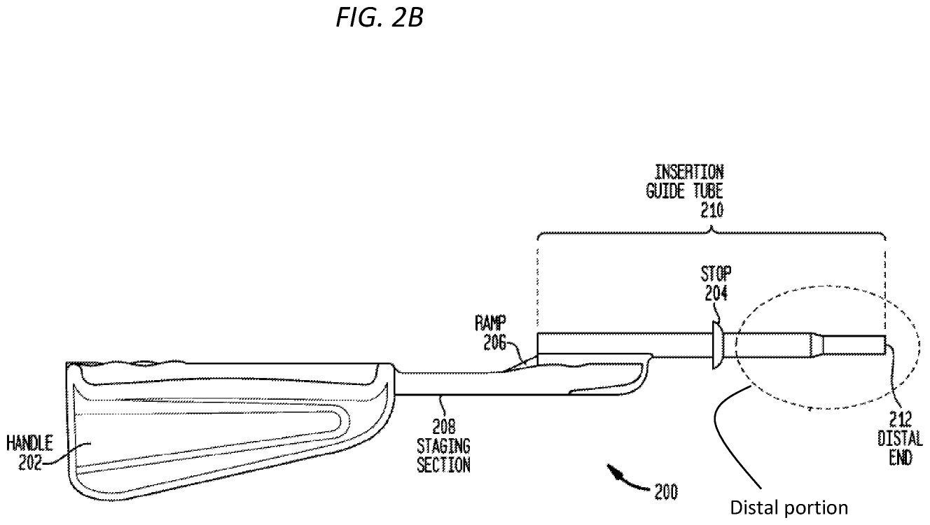

FIG. 2A presents a side view of an embodiment of an insertion guide for implanting an elongate electrode assembly generally represented by electrode assembly 145 (corresponding to assembly 190 of FIG. 1B) into a mammalian cochlea, represented by cochlea 140. The illustrative insertion guide, referred to herein as insertion guide 200, includes an elongate insertion guide tube 210 configured to be inserted into cochlea 140 and having a distal end 212 from which an electrode assembly is deployed. Insertion guide tube 210 has a radially-extending stop 204 that may be utilized to determine or otherwise control the depth to which insertion guide tube 210 is inserted into cochlea 140.

Insertion guide tube 210 is mounted on a distal region of an elongate staging section 208 on which the electrode assembly is positioned prior to implantation. A robotic arm adapter 202 is mounted to a proximal end of staging section 208 to facilitate attachment of the guide to a robot, which adapter includes through holes 203 through which bolts can be passed so as to bolt the guide 200 to a robotic arm, as will be detailed below. During use, electrode assembly 145 is advanced from staging section 208 to insertion guide tube 210 via ramp 206. After insertion guide tube 210 is inserted to the appropriate depth in cochlea 140, electrode assembly 145 is advanced through the guide tube to exit distal end 212 as described further below.

FIG. 2B depicts an alternate embodiment of the insertion guide 200, that includes a handle 202 that is ergonomically designed to be held by a surgeon. This in lieu of the robotic arm adapter 202.

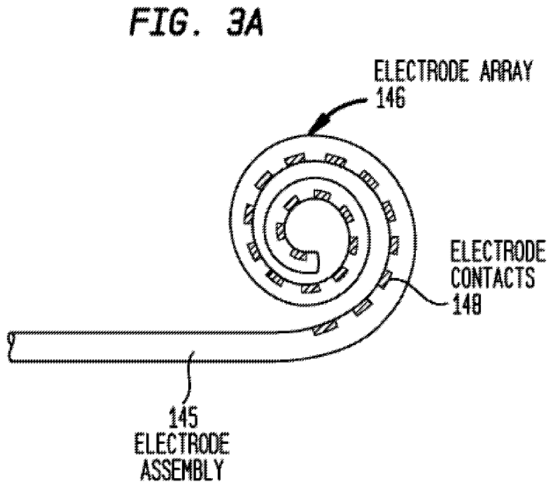

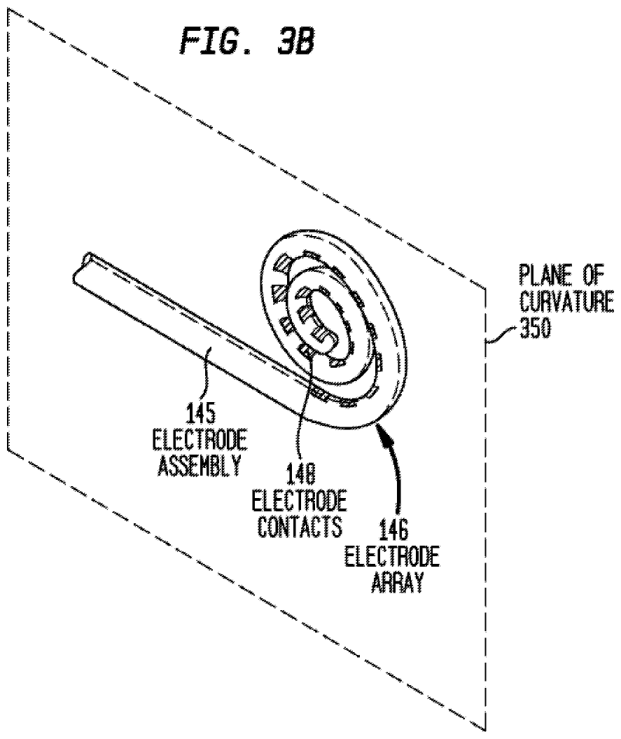

FIGS. 3A and 3B are side and perspective views, respectively, of representative electrode assembly 145. As noted, electrode assembly 145 comprises an electrode array 146 of electrode contacts 148. Electrode assembly 145 is configured to place electrode contacts 148 in close proximity to the ganglion cells in the modiolus. Such an electrode assembly, commonly referred to as a perimodiolar electrode assembly, is manufactured in a curved configuration as depicted in FIGS. 3A and 3B. When free of the restraint of a stylet or insertion guide tube, electrode assembly 145 takes on a curved configuration due to it being manufactured with a bias to curve, so that it is able to conform to the curved interior of cochlea 140. As shown in FIG. 3B, when not in cochlea 140, electrode assembly 145 generally resides in a plane 350 as it returns to its curved configuration. That said, it is noted that embodiments of the insertion guides detailed herein and/or variations thereof can be applicable to a so-called straight electrode array, which electrode array does not curl after being free of a stylet or insertion guide tube, etc., but instead remains straight.

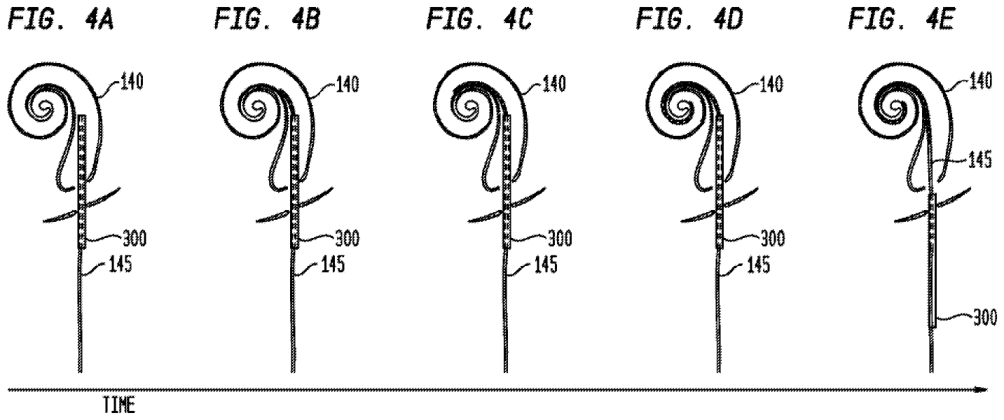

FIGS. 4A-4E are a series of side-views showing consecutive exemplary events that occur in an exemplary implantation of electrode assembly 145 into cochlea 140. Initially, electrode assembly 145 and insertion guide tube 310 are assembled. For example, electrode assembly 145 is inserted (slidingly or otherwise) into a lumen of insertion guide tube 300. The combined arrangement is then inserted to a predetermined depth into cochlea 140, as illustrated in FIG. 4A. Typically, such an introduction to cochlea 140 is achieved via cochleostomy 122 (FIG. 1) or through round window 121 or oval window 112. In the exemplary implantation shown in FIG. 4A, the combined arrangement of electrode assembly 145 and insertion guide tube 300 is inserted to approximately the basal turn of cochlea 140.

As shown in FIG. 4A, the combined arrangement of insertion guide tube 300 and electrode assembly 145 is substantially straight. This is due in part to the rigidity of insertion guide tube 300 relative to the bias force applied to the interior wall of the guide tube by pre-curved electrode assembly 145. This prevents insertion guide tube 300 from bending or curving in response to forces applied by electrode assembly 145, thus enabling the electrode assembly to be held straight, as will be detailed below.

As noted, electrode assembly 145 is biased to curl and will do so in the absence of forces applied thereto to maintain the straightness. That is, electrode assembly 145 has a memory that causes it to adopt a curved configuration in the absence of external forces. As a result, when electrode assembly 145 is retained in a straight orientation in guide tube 300, the guide tube prevents the electrode assembly from returning to its pre-curved configuration. This induces stress in electrode assembly 145. Pre-curved electrode assembly 145 will tend to twist in insertion guide tube 300 to reduce the induced stress. In the embodiment configured to be implanted in scala tympani of the cochlea, electrode assembly 145 is pre-curved to have a radius of curvature that approximates the curvature of medial side of the scala tympani of the cochlea. Such embodiments of the electrode assembly are referred to as a perimodiolar electrode assembly, and this position within cochlea 140 is commonly referred to as the perimodiolar position. In some embodiments, placing electrode contacts in the perimodiolar position provides utility with respect to the specificity of electrical stimulation, and can reduce the requisite current levels thereby reducing power consumption.

As shown in FIGS. 4B-4D, electrode assembly 145 may be continually advanced through insertion guide tube 300 while the insertion sheath is maintained in a substantially stationary position. This causes the distal end of electrode assembly 145 to extend from the distal end of insertion guide tube 300. As it does so, the illustrative embodiment of electrode assembly 145 bends or curves to attain a perimodiolar position, as shown in FIGS. 4B-4D, owing to its bias (memory) to curve. Once electrode assembly 145 is located at the desired depth in the scala tympani, insertion guide tube 300 is removed from cochlea 140 while electrode assembly 145 is maintained in a stationary position. This is illustrated in FIG. 4E.

Conventional insertion guide tubes typically have a lumen dimensioned to allow the entire tapered electrode assembly to travel through the guide tube. Because the guide tube is able to receive the relatively larger proximal region of the electrode assembly, there will be a gap between the relatively smaller distal region of the electrode assembly and the guide tube lumen wall. Such a gap allows the distal region of the electrode assembly to curve slightly until the assembly can no longer curve due to the lumen wall.

Returning to FIGS. 3A-3B, perimodiolar electrode assembly 145 is pre-curved in a direction that results in electrode contacts 148 being located on the interior of the curved assembly, as this causes the electrode contacts to face the modiolus when the electrode assembly is implanted in or adjacent to cochlea 140. Insertion guide tube 300 retains electrode assembly 145 in a substantially straight configuration, thereby preventing the assembly from taking on the configuration shown in FIG. 3B.

It is noted that while the embodiments above disclose the utilization of an insertion tool, in some other embodiments, insertion of the electrode array is not executed utilizing an insertion tool. Moreover, in some embodiments, when in insertion tool is utilized, the insertion tool is not as intrusive as that detailed in the figures. In an exemplary embodiment, there is no distal portion of the tool. That is, the insertion tool stops at the location where the distal portion begins. In an exemplary embodiment, the tool stops at stop 204. In this regard, there is little to no intrusion of the tool into the cochlea. Any device, system and/or method that can enable the insertion of the electrode array can be utilized in at least some exemplary embodiments.

As can be recognized from the above, the electrode array can be utilized to obtain the data utilized in the methods herein, such as by way of example only and not by way of limitation, the voltages at the read electrodes, and can also be used to provide the stimulating electrode (just in case for some reason that was not clear). FIG. 5 depicts an exemplary system for utilizing the cochlear implant to obtain such information. Presented in functional terms, there is a test unit 3960 in signal communication with unit 8310, which in turn is in signal communication, optionally with a unit 7720 and a unit 8320, the details of which will be described below.

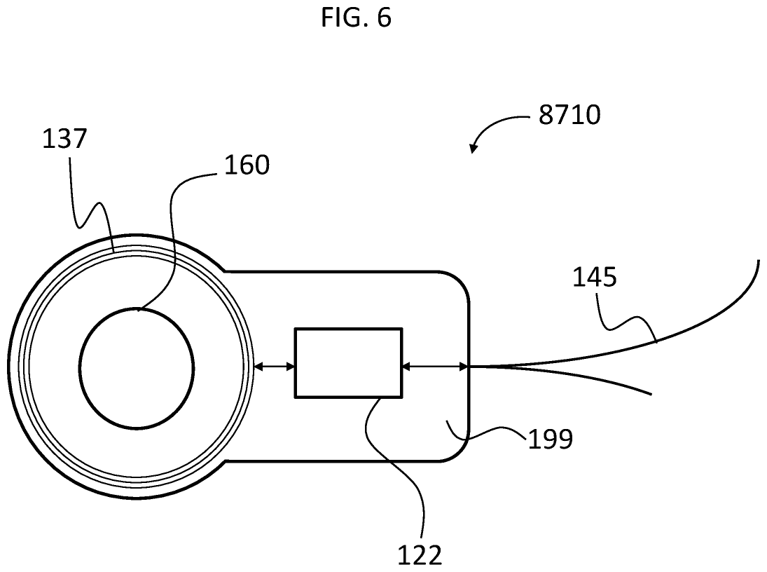

Unit 3960 can correspond to an implantable component of an electrode array, as seen in FIG. 1. More specifically, FIG. 6 depicts an exemplary high-level diagram of a receiver/stimulator 8710 (the implantable portion of 100) of a cochlear implant, looking downward. As can be seen, the receiver/stimulator 8710 includes a magnet 160 that is surrounded by a coil 137 that is in two-way communication (although in other embodiments, the communication is one-way) with a stimulator unit 122, which in turn is in communication with the electrode array 145. Receiver/stimulator 8710 further includes a cochlear stimulator unit 122, in signal communication with the coil 137. The coil 137 and the stimulator unit 122 are encased in silicon as represented by element 199. In an exemplary embodiment, receiver/stimulator 8710 is utilized as test unit 3960, and is used to acquire information about electrode array position.

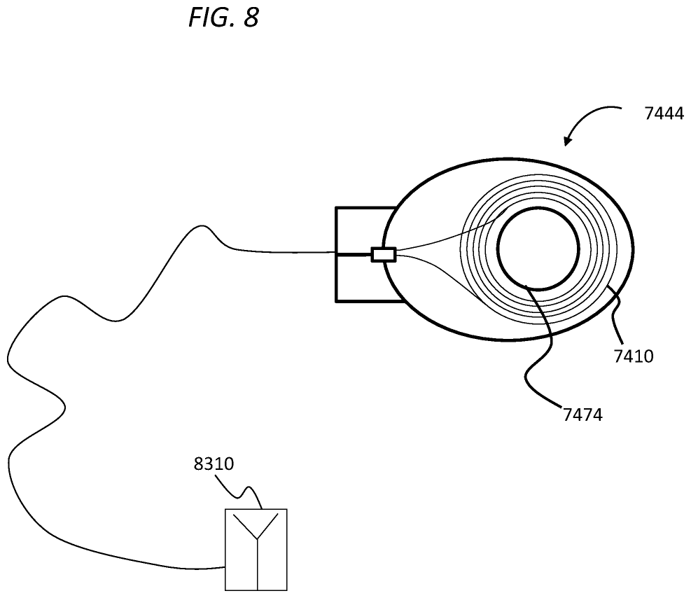

FIG. 8 depicts an exemplary RS (receiver/stimulator) interface 7444 which is presented by way of concept. An inductance coil 7410 is configured to establish a magnetic inductance field so as to communicate with the corresponding coil of the receiver-stimulator of the cochlear implant. Interface 7444 includes a magnet 7474 so as to hold the inductance coil 7410 against the coil of the receiver/stimulator of the cochlear implant in a manner analogous to how the external component of the cochlear implant is held against the implanted component, and how the coils of those respective components are aligned with one another. As can be seen, an electrical lead extends from the coil 7410 to control unit 8310, representing signal communication between interface 7444, and control unit 8310. It is noted that in an alternative embodiment, 7444 can be the external component of FIG. 1, and can have some and/or all of the functionalities just described, such that data can be obtained from the implanted portion outside of a clinical setting, such as during everyday life of the recipient.

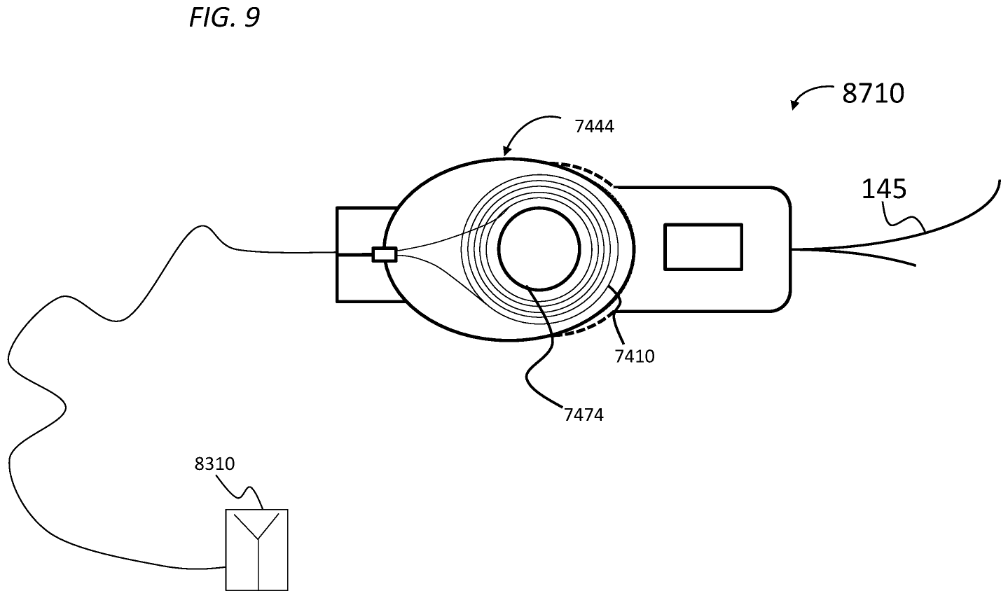

FIG. 9 depicts an exemplary embodiment of the receiver/stimulator 8710 in signal communication with the control unit 8310 via electrical lead that extends from the interface device 7444 having coil 7410 about a magnet 7474 as can be seen. The interface device 7444 communicates via an inductance field with the inductance coil of the receiver/stimulator 8710 so that the data acquired by the implantable component 8710 (receiver/stimulator) can be transferred to the control unit 8310.

Note also that in at least some alternate exemplary embodiments, control unit 8310 can communicate with the so-called “hard ball” reference electrode of the implantable component of the cochlear implant so as to enable communication of data from the receiver/stimulator 8710 to control unit 8310 and/or vice versa.

It is noted that in the embodiment of FIG. 9, control unit 8310 is in signal communication with the various other components as detailed herein, which components are not depicted in FIG. 9 for purposes of clarity.

Also functionally depicted in FIG. 5 is the optional embodiment where an electrode array insertion robotic system/actuator system 7720 and an input device 8320 is included in the system. In an exemplary embodiment, the input device 8320 could be a trigger of a handheld device that controls the actuator system 7720 and can stop and/or start the actuator for insertion of the electrode array. In an exemplary embodiment, the input device 8320 could be a trigger on the tool 8200.

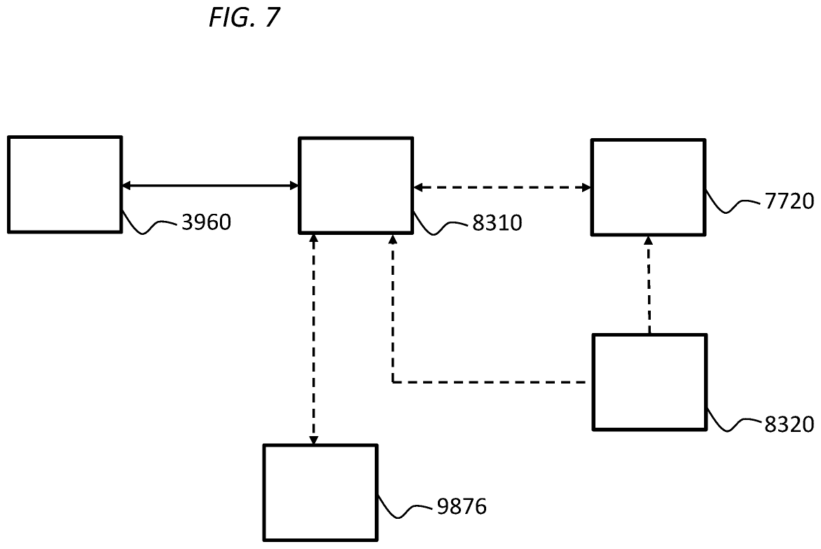

Control unit 8310 can be a signal processor or the like, or a personal computer or the like, or a mainframe computer or the like, etc., that is configured to receive signals from the test unit 3960 and analyze those signals to evaluate the data obtained (it can also be used to control the implant/control the application of current). More particularly, the control unit 8310 can be configured with software or the like to analyze the signals from test unit 3960 in real time and/or in near real time as the electrode array is being advanced into the cochlea by actuator assembly 7720 (if present, and if not present, while the array is being inserted/advanced by hand). The control unit 8310 analyzes the input from test unit 3960, after partial and/or full implantation and/or after the surgery is completed and/or as the electrode array advanced by the actuator assembly 7720 and/or as the electrode array is advanced by the surgeon by hand. The controller/control unit can be programmed to also control the stimulation/control the providing of current to the electrodes during the aforementioned events/situations. The controller 8310 can evaluate the input to determine if there exists a phenomenon according to the teachings detailed herein. The controller can evaluate telemetry, or otherwise receive telemetry, form the implant, via the device that communicates with the implant. That said, in an alternate embodiment, as depicted in FIG. 7, or in addition to this, the controller 8310 can output a signal to an optional monitor 9876 or other output device (e.g., buzzer, light, etc.), that can provide the surgeon or other healthcare professional performing the operation or evaluating the data postoperatively, etc., indicative of the data obtained and/or indicative of a conclusion reached by the control unit 8310. Note also that in an exemplary embodiment, the control unit 8310 can be a dumb unit in the sense that it simply passes along signals to the implant (e.g., the control unit can instead be a series of, for example, buttons where a surgeon depression is one button to provide stimulation to a given electrode). The control unit 8310 can be an external component of the cochlear implant.

Still, in some embodiments, the control unit 8310 is configured or otherwise programmed to evaluate input and determine if the input indicates that the electrode array is positioned in a given manner were otherwise that the electrode array is positioned in a manner different than that which was desired or otherwise determine any of the features detailed herein. In an exemplary embodiment, upon such a determination, control unit 8310 could halt the advancement of the array into the cochlea by stopping the actuator(s) of actuator assembly 7720 and/or could slow the actuator(s) so as to slow rate of advancement of the electrode array into the cochlea and/or could reverse the actuator(s) so as to reverse or otherwise retract the electrode array within the cochlea (either partially or fully). Alternatively, in embodiments where actuator assembly 7720 is not present, control unit 8310 could provide an indication to the surgeon or the like (via an integrated component, such as a buzzer or a light on the control unit, or an LDC screen, or via device 9876) to halt and/or slow the insertion, etc. In at least some exemplary embodiments, control unit 8310 can be configured to override the input from input unit 8320 input by the surgeon or the user.

Some exemplary embodiments utilize the receiver/stimulator 8710 as a test unit 3910 that enables the action of obtaining the data and the action of providing current to the electrode, and/or any one or more of the method actions detailed herein. In an exemplary embodiment, the receiver/stimulator 8710 and/or control unit 3810 and/or actuator assembly 7720 and/or input device 8320 are variously utilized to execute one or more or all of the method actions detailed herein, alone or in combination with an external component of a cochlear implant, and/or with the interface 7444, which can be used after the receiver/stimulator 8710 is fully implanted in the recipient and the incision to implant such has been closed (e.g., days, weeks, months or years after the initial implantation surgery). The interface 7444 can be used to control the receiver/stimulator to execute at least some of the method actions detailed herein (while in some other embodiments, the receiver/stimulator can execute such in an autonomous or semi-autonomous manner, without being in communication with an external component) and/or can be used to obtain data from the receiver/stimulator after execution of such method actions.

Some exemplary utilizations of the embodiments of FIGS. 5-9 will now be described, along with some modifications thereto.

In view of the above, some embodiments include the utilization of, for example, one or more of the systems detailed above and/or below, to obtain electrode voltage measurements along the electrode array inserted into the cochlea. Some embodiments also include an analysis, such as an automatic analysis and/or semiautomatic analysis, such as by the systems detailed above, of the electrode voltages to determine one or more of the features herein/practice one or more of the method actions herein. Embodiments herein disclosed as using four point impedance measurements include using one or more of the devices above to execute such and to communicate the results to the outside and/or to a clinician or other healthcare professional. Any disclosure herein of implementing impedance tests includes a disclosure of using the above devices and using any one or more of the above noted devices' functionalities. And in some embodiments, the above noted devices are configured to execute one or more of the method actions herein.

Bipolar stimulation of a cochlear implant, such as where two electrodes of the electrode array implanted in the cochlea or otherwise in the cochlea at the time that bipolar stimulation is executed are respectively utilized as the source and a sink, produces a dipole within the cochlea. The quantity of current flowing out of a current source is at least effectively equal (including equal) to the current flowing back into the current source, in some embodiments where the source and sink are located in a cochlea with perilymph immersing both the source and a sink in a contiguous manner. In at least some exemplary embodiments, the cochlear implant electrode array 146 provides stimulation to tissue utilizing a current source and sink established by two electrodes of the electrode array. In an exemplary embodiment, this current source provides sufficient current into tissue of the recipient to evoke a hearing percept. In some embodiments according to the teachings detailed herein, irrespective of whether or not a hearing percept is executed, although some embodiments apply a current that creates current flow within the cochlea where no hearing percept is evoked and/or the threshold level at a given frequency for that recipient is higher than the current utilized, while in other embodiments, a hearing percept is evoked, the current flow generated by the stimulating electrodes will flow according to an impedance within the cochlea.

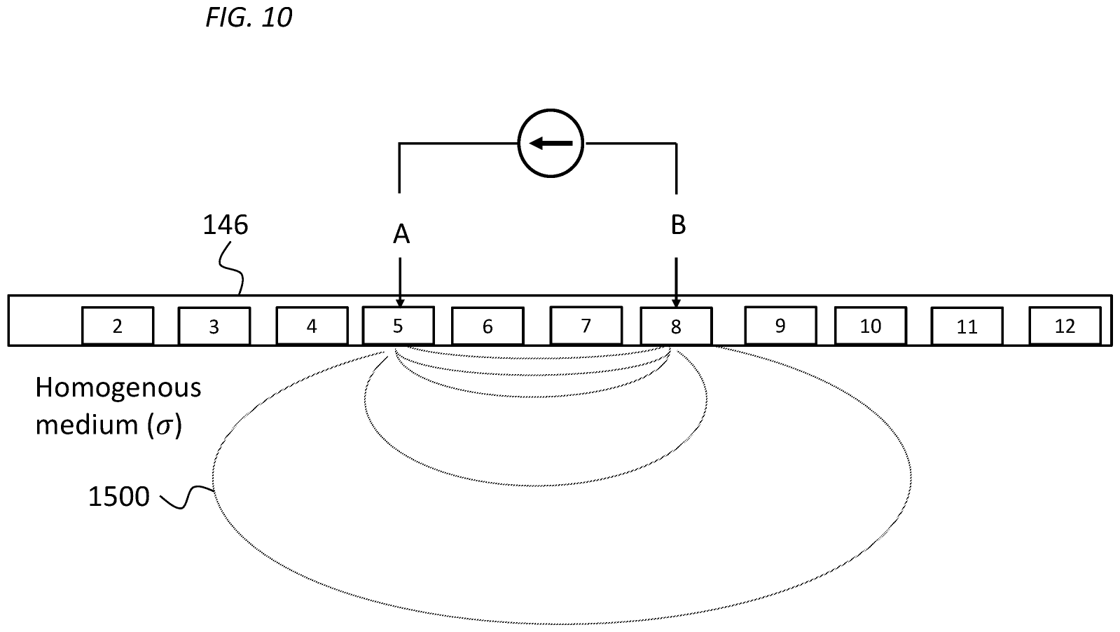

FIG. 10 shows an exemplary conceptual diagram of current flow between a source and a sink of an electrode array within the cochlea. Thus if the relative voltage is measured between ICE6 (ICE being intra-cochlear electrode—as distinguished from an electrode that is not in the cochlea, at least not when the measurement is executed) and ICE7, where electrodes 5 and 8 (ICE electrodes 5 and 8) are the stimulating electrodes (source and sink), in some exemplary scenarios, the voltage difference would be a certain value with respect to a cochlea in which no blood is present and another value (a higher value, in at least some instances), where there is blood in the cochlea. Thus, FIG. 10 depicts a 4-point impedance measurement regimes. By using such measurement regimes, differences between the 4-point (bulk) measures due to different biological mediums can be detected, and used to utilitarian value.

Embodiments include a multi-contact cochlea electrode array, such as those detailed above, an implant with extra-cochlear electrodes (or another component, such as one that works in conjunction with the implanted portion of the cochlear implant, a receiver stimulator (such as that of the implanted portion), which can be either fully implanted or powered by an external behind the ear (BTE) processor or other external device. The implanted portion can include a built in-built amplifier configured to measure electrode voltages concurrent to the delivery of electrical current to either the same or adjacent electrode contacts.

In some instances, a method is executed whereby the implanted portion of the cochlear implant (e.g., receiver stimulator) or another device, such as the control unit detailed above, coordinates measurement electrode voltages in response to electrical stimulation to one or more contacts such that a measure of bulk impedance between two electrode contacts can be estimated. This bulk impedance is calculated for a number of locations along the electrode array in some embodiments. These measurements may be repeated continuously over time, either during the insertion of the electrode array or post-insertion when the electrode array is static in position (either during the surgery or post-operatively).

Methods further include detection and analysis, wherein, in some embodiments, the system or external apparatus (BTE or computer/control unit, for example) analyzes the measured impedances/measured voltages along the array (at one or more or all possible locations) to determine one or more of the features determined below.

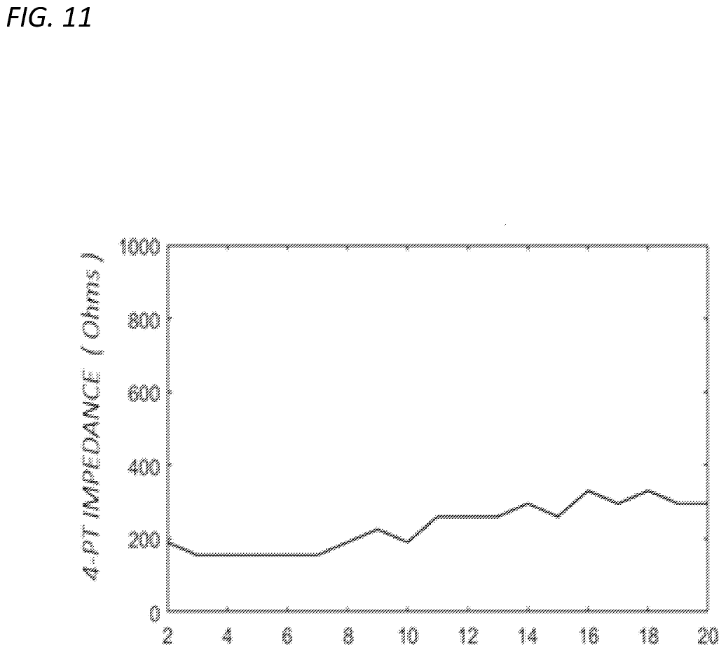

FIG. 11 depicts an exemplary graph of impedance between read electrodes. The graphs depict the impedances between two electrodes, where the number presents the electrode with the lower number (i.e., 2 represents the read pair of electrodes 2 and 3, 20 represents the read pair of electrodes 20 and 21, etc.). This number is referred to as the base electrode (for no other reason than to establish a standard—if the base electrode was the higher number, the charts would start with 3 and end with 21, but the data would be the same). This can be an example of an impedance measurement taken at the time of implantation. For example, FIG. 11 can represent measurements immediately after the electrode insertion in the operating room and/or measurements from a time period less than 10 minutes (e.g., less than 1, 2, 3, 4, 5, 6, 7, 8, 9, or 10 minutes) after the initial inserting of the electrode array.

Embodiments include using one or more sets of impedance measurements taken at specific temporal locations and/or separate temporal intervals, as latent variables to identify, or at least determine the plausibility, that one or more deleterious scenarios have occurred and/or will occur within the cochlea as a result of insertion of a cochlear implant electrode array therein, and to commence a treatment based on the impedance measurements. In some embodiments, there is an increase in impedance within a 24 hour period, but this period can be different in other embodiments as will be detailed further below. Embodiments include utilizing the impedance measurements to detect biomarkers (again, by way of latent variables in some embodiments) that are indicative of a change within a cochlea that will result in increased impedance between electrodes of a cochlear implant temporally downrange, which increase in impedance, if permitted to occur, is sufficiently high that the increase will have a deleterious effect on the operation of the cochlear implant (such as requiring an increase in power of the implant to evoke the same hearing percept as that which would be the case in the absence of the increase—in an embodiment, the increase in impedance can result in an increased load on the cochlear implant of at least 10, 15, 20, 25, or 30%, or any value or range of values therebetween in 1% increments above that which would otherwise be the case, all other things being equal). Embodiments further include utilizing the impedance measurements to determine impedance between at least two electrodes, in some embodiments, electrodes immediately adjacent to each other, and forecasting potential dizziness resulting from implantation of the electrode array. More on this below.

In some embodiments, a foreign body reaction after cochlear implantation is dependent on several factors including but not limited to intra-cochlear injury during electrode array insertion, and surgical approach. Electrode array insertion can be a source of physical trauma to the cochlea. An exemplary physical trauma can be fracture of the osseous spiral lamina and displacement of the basilar membrane for perimodiolar electrode arrays. Some embodiments herein include experiencing such during the implantation of the electrode array, wherein the experience causes or at least creates a cascade of reactions that ultimately result in the detected impedance difference. These traumas can trigger cell-death pathways such as necrosis, which can cause the down-range rise in impedance, making the cochlear implant more difficult to operate. These traumas can occur in some embodiments, and treatments herein keyed off of the difference in the impedance can limit the severity and/or prevent such. This can be a passive cell death mechanism initiated by substantial physical trauma to a cell. Necrotic cell death involves cell swelling leading to cell lysis, with the intra-cellular content released into the perilymphatic space and eliciting an extensive acute inflammatory response. (In some embodiments, this can occur, and the teachings herein can include limiting and/or preventing such.) This involves the influx of inflammatory and reparatory cells such as macrophages, leukocytes and neutrophils into the cochlea. This influx can, in some embodiments, cause the rise in the impedance at the approximate one day timeframe, and thus the rise in impedance can be an indicator that this has happened, and thus the rise in impedance can be an indicator that treatment should commence. Necrosis-induced sensory cell death can occur after cochlear implantation resulting in hearing loss. Embodiments are directed to reducing and/or limiting this occurrence.

Following this hearing loss, further hearing loss can occur after cochlear implantation, transpiring up to 7 days post implantation for example, and embodiments are directed towards limiting and/or avoiding this scenario. This hearing loss can occur even in the absence of physical trauma to the macroscopic elements of the cochlea. Embodiments include executing the teachings herein in a cochlea that has the absence of physical trauma to the macroscopic elements.

In some scenarios, there can be oxidative stress in cochlea cells following electrode insertion, which may lead to apoptosis in damaged hair cells and trigger an acute inflammatory response. Hair cell death via apoptosis can progressively increase 12, 24, and 36 hours after implantation. Thus, molecular damage may be caused from electrode insertion but not a direct consequence of physical trauma. Potential causes for this damage include hydraulic trauma during electrode insertion, whereby changes in perilymph volume have been correlated to hearing loss severity. Additionally, perimodiolar implants are sometimes used with the exo-sheath (as noted above) to assist in the placement of the array in the cochlea. Insertion of the sheath may induce large intra-cochlear pressure changes that may be traumatic. All of these scenarios can occur in some embodiments of the implantation of the cochlear implant. Embodiments include using the impedance measurements as latent variables to identify one or more of the above occurrences, or at least indicate that one or more of the above occurrences possibly occurred, and thus to commence treatment.

Methods include energizing one or more electrodes of a cochlear electrode array to induce a current flow in the cochlea at a plurality of temporal locations to execute four point impedance measurement. By way of example only and not by way of limitation, in an exemplary embodiment, this can include energizing electrode 9 and/or 12 (where 9 and 12 are alternatingly utilized as a source and sink when both are energized). Other electrode(s) can be energized. In some exemplary embodiments, the electrodes that are energized are electrodes that are before, as utilitarian as possible, the basal turn (keeping in mind that there can be utility with respect to having the read electrodes before basal turn). Any energizement regime that can enable the teachings detailed herein can be utilized in at least some exemplary embodiments. Note further that implementation of four point impedance can include energizing different pairs of the electrodes in a temporally spaced apart manner but where collectively, the energizement corresponds to a single temporal location.

Four-point impedance can include measuring one or more electrical properties at one or more locations in the cochlea resulting from the induced current flow at the plurality of different temporal locations.

In an embodiment, the impedance measurements used for the first temporal location are for a fully seated electrode array/after the electrode is fully inserted into the cochlea.

In an exemplary embodiment, the measurement electrodes utilized are those that are located before and/or at and/or proximate the basal turn, and the other electrodes are not utilized, at least not effectively (perhaps some instances all of the electrodes are utilized, but the bulk of the data collection is based on only those electrodes before the location). In an exemplary embodiment, more than 20, 25, 30, 35, 40, 45, 50, 55, 60, 65, 70, 75, 80, 85, 90, 91, 92, 93, 94, 95, 96, 97, 98, or 99% of the data collected and/or utilized to execute one or more of the method actions herein comes from electrodes that are at and/or before the basal turn.

In an exemplary embodiment, the electrodes that are used as read electrodes and/or energized electrodes and/or electrodes that are utilized as read electrodes in the method actions herein with respect to evaluating the impedances detailed herein (e.g., values might be obtained for all the electrodes are more than these electrodes, but the analysis of the trans impedance data is limited to data from these electrodes) and/or the electrodes used to perform the comparisons/evaluations herein fall within a distance of no closer than 0.5, 0.75, 1, 1.25, 1.5, 1.75, 2, 2.5, 3, 3.5, 4, 5, 6, 7, 8, 9, 10, 11, 12, 13, 14, or 15 millimeters from a line taken normal to a tangent at the beginning of the first basal turn at the lateral wall (and are thus between the most proximal portion of the cochlea and that line). In an embodiment, the electrodes that are utilized/the electrodes from which the data is utilized for the comparisons/evaluations herein are located no more than 8, 9, 10, 11, 12, 13, 14, 15, 16, or 17 mm from the proximal portion of the cochlea where the electrode array enters the cochlea (as measured along the length of the array) and/or are no less than 2, 3, 4, 5, 6, 7, 8, 9, or 10 mm from that location.

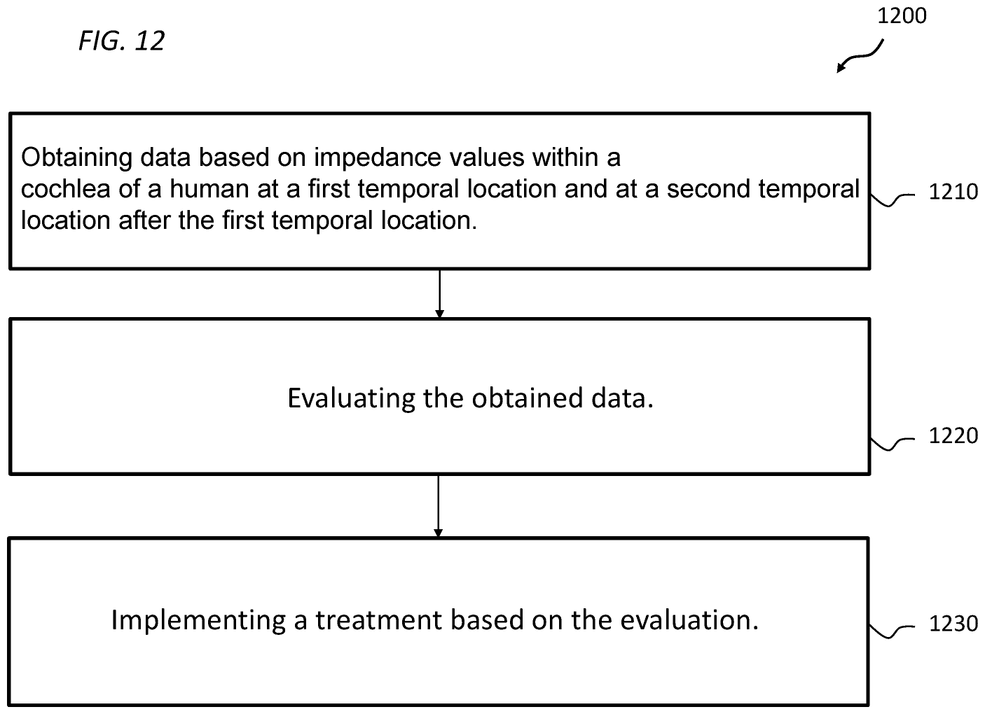

FIG. 12 presents an exemplary flowchart for an exemplary method, method 1200, according to an exemplary embodiment. Method 1200 includes method action 1210, which includes obtaining data based on impedance values within a cochlea of a human at a first temporal location and at a second temporal location after the first temporal location. In an embodiment, the first and second temporal locations are separated by less than 30 hours and more than 12 hours. In an embodiment, the first and second temporal locations are separated by at least (inclusive) 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, or 50 hours, or any value or range of values therebetween in 0.1 hour increments (e.g., 20.3 hours, 33.1 hours, 22.3 to 25.4 hours, etc.). In an embodiment, the first and second temporal locations are separated by no more than (inclusive) 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, or 70 hours or any value or range of values therebetween in 0.1 hour increments (e.g., 15.3 hours, 28.1 hours, 20.7 to 28.8 hours, etc.).

In an exemplary embodiment, the action of obtaining data based on impedance values within a cochlea of human can include actually operating the cochlear implant electrode array utilizing four point impedance measurements. In an exemplary embodiment, the action of obtaining data based on impedance values within a cochlea of a human can include receiving the values from a third party, such as from a hospital that is remote from a data center or remote from a clinician or inner ear specialist who is reviewing the data (and thus executing the method). Thus, data based on impedance values can be the impedance values or can be a data set that is compiled based on impedance values or data that is compiled based on data that is compiled based on impedance values.

In an exemplary embodiment, the data can include impedance values for all or some of the read electrodes at a first temporal location. The data can include impedance values for all or some of the read electrodes at a second temporal location. It is noted that the impedance values for all of the read electrodes need not necessarily be present in the data set. Indeed, in an exemplary embodiment, a data set for the first temporal location may or may not have one or more impedance values for one or more read electrodes whereas the data set for the second temporal location may have one or more those impedance values for the read electrodes. That is, the data sets need not necessarily be exhaustive or otherwise identical providing that the data obtained can enable the teachings detailed herein.

In an exemplary embodiment, the obtained data can be a mean value of the impedances for all or some of the read electrodes (some embodiments focus on only the basal electrodes—more on this below). In an embodiment, the obtained data can be the raw impedances for each of the read electrodes. The obtained data can be a combination of both. The obtained data need not necessarily be obtained at the time that the impedance values were measured. That is, method action 1210 can occur hours or even days after the first and second temporal locations.

Method 1200 includes method action 1220, which includes the action of evaluating the obtained data. And the data obtained can be more than the data used in the actions of comparing or evaluating, etc. This does not require evaluating all of the obtained data, although some embodiments will include evaluating all of the obtained data.

In an embodiment, this can entail comparing the mean value of the impedances for the impedance values within the cochlea of the human at the first temporal location with the mean value of the impedances obtained at the second temporal location. In an embodiment, instead of the mean value, the median value can be utilized. Even more sophisticated statistical analysis/comparisons can be utilized in method action 1220 (or the data in method action 1210 can be the result of such analysis with respect to the values at the first and second temporal locations, providing that the data can be evaluated).

In an embodiment, the action of evaluating the obtained data in method action 1220 can include comparing the obtained data to determine whether or not the mean average of the impedance values at the first temporal location are greater than or less than or statistically the same as the mean average of the impedance values at the second temporal location. As will be described in greater detail below, owing to the work of the inventors, it has been determined that impedance differences of a certain amount between two temporal locations (such as locations separated by the time periods detailed above) can indicate future deleterious effects within a cochlea. The additional features of this will be described in greater detail below. For the moment, it has been determined an increase in the mean impedance values of 10% or more indicates a high likelihood that future changes in the cochlea will take place which will result in an increase in the impedance within the cochlea (such as relative to the environment that existed at the time of implantation of the electrode array) that will have a deleterious effect on the operation of the cochlear implant and/or can result in the loss of some or all of the residual hearing of the recipient.

In an embodiment, the action of evaluating the obtained data can be based on statistical data in addition to the data based on impedance values. By way of example only and not by way of limitation, the action of evaluating the obtained data can take into account the age and/or the sex of the recipients. By way of example only and not by way of limitation, an increase of 5% in the mean impedance values might be indicative of a need for therapeutic intervention or otherwise that there can be utilitarian value with respect to implementing a therapeutic intervention for age groups below 50 years old, whereas such an increase might not be indicative of a need for therapeutic intervention or otherwise that there can be utilitarian value with respect to implementing a therapeutic intervention for age groups older than 50 years old. Statistical data for such statistical groups can be compiled to further refine the action of evaluating the obtained data.

And this leads to method action 1230, which includes the action of implementing a treatment based on the evaluation. Here, if the increase in the mean impedance values determined in method action 1220 shows, for example, a 12% increase over a 24 hour time period, a treatment regime based on applying, for example, steroids to the recipient can be implemented. In an exemplary embodiment, the treatment regime is a treatment regime that, statistically speaking, could have deleterious effects that might outweigh the beneficial effects. For example, steroids and clot busters are not routinely prescribed after each cochlear implant surgery because of the potential side effects. Here, based on the evaluation of the obtained data, a prediction can be made that in the absence of treatment, certain negative effects are likely to occur, where the likelihood of such is sufficiently high so as to outweigh the negative effects of implementing the treatment. Put another way, method actions 1210 and 1220 provide a method to prescribe further treatment systemically with justified risks. These method actions also provide a method to prescribe further treatment locally, such as some controlled-release system in the implant, or a middle ear intervention by way of example only and not by way of limitation. And note that these are just a few of the examples of the types of prescriptive interventions that can be used. Additional prescriptive interventions will be described in greater detail below in a nonexhaustive manner. The point here is that by obtaining the impedance values within/at certain period of times and applying a statistical analysis thereto, a risk balance evaluation can be executed that would permit a therapy to be ethically (ranging from the wanton prescribing of drugs/implementation of therapy to the concept of defensive medicine) and/or otherwise less blindly applied than otherwise would be the case.

It is briefly noted that while many of the methods herein present implementing a treatment based on the evaluations/comparisons of the data from the four point impedance, embodiments can also include, in addition to this or alternatively to this, recommending a treatment therapy/prescribing a therapy and/or controlling a medical device, such as an implantable medical device and/or developing and/or providing a prognosis/executing a prognostic action based on the comparison/evaluation. Any disclosure herein of one corresponds to an alternate disclosure of any one or more of the other in the interests of textual economy providing that the art enables such unless otherwise noted.

Thus, it can be seen that in an embodiment of method 1200, the evaluation of the obtained data indicates an increase in impedance values from the first temporal location to the second temporal location and the action of implementing the treatment is an action that would not be executed if the evaluation did not indicate the increase. And in some embodiments, this increase must be above/at a certain level, which level can be variable depending on the statistical classification into which the person/recipient falls.

In an embodiment, the obtained data indicates an increase in impedance values (e.g., the mean impedance values), at least some impedance values of interest (e.g., the values of the read electrodes before the basal turn, or the read electrodes detailed herein, etc., by at least 4, 5, 6, 7, 8, 9, 10, 11, 12, 13, 14, or 15% or more, or any value or range of values therebetween in 0.1% increments, and the value can vary depending on the statistical background of the recipient (age, race, sex, extent of hearing loss by way of example only and not by way of limitation). For example, a recipient that has total hearing loss might be set at a level of a 7.5% increase, whereas one with residual hearing might be set at 5%. The idea here is that the recipient likely has all of his or her hair cells in the cochlea in a dead state and thus the introduction of the cochlear implant electrode array is less likely to cause a reaction that will result in the impedance increases over the aforementioned temporal periods, and thus any such increases should be more cautiously evaluated than that which would be the case for a person with residual hearing, where a higher level of an increase in impedance values might be expected (all without the down arrange deleterious effects with respect to the ultimate increase in impedance within the cochlea). To be clear, in an exemplary embodiment, if the obtained data indicates an increase in the impedance values by any of the above noted values (which value is selected depending on the embodiment), the treatment would be implemented, and if the obtained data indicates an increase in the impedance by less than any of the above noted values, the treatment would not be implemented. Alternatively, in an embodiment, the magnitude of the treatment could be varied depending on the percentage change as will be detail below.

Still, as will be detailed below, studies indicate that an increase in the mean impedance values by at least 10% from the first temporal location to the second temporal location, at least when taking into account at least 30, 35, 40, 45, 50, 55, 60, 65, 70, 75, or 80% of the read electrodes (the values from those read electrodes—again, some read electrodes will not be used for various reasons) or any value or range of values therebetween in 1% increments is cause for implementation of one or more of the therapeutic treatments detailed herein or other therapeutic treatments.

As noted above, the first temporal location is separated by the second temporal location by, in some embodiments, no more than 36 hours in some embodiments. For ease of convenience, at a minimum, the first and second temporal periods should be separated by 24 hours for example (plus or minus 1 or 2 or 3 or 4 hours for example) or the second location should occur before standard close of business times the day following the first temporal location. Thus, in theory, if the first temporal location existed at, for example, 9 a.m. on a Monday and the second location existed at 5 p.m. on the immediately following Tuesday, the time difference would be 32 hours. In embodiments, the time spacing could be later (e.g., 10:00 p.m. on the immediately following Tuesday, after a change to the late shift at a hospital). Embodiments can include a scenario where if the first temporal location existed at, for example, 7 p.m. on a Monday, the second temporal location could exist at, for example, 7 a.m., on the following Wednesday, and thus a full day separates the two temporal locations.

In an embodiment, the first and second temporal locations are separated by no more than 36 hours, 48 hours, 60 hours, and in some embodiments, no more than 72 hours.

In an embodiment, concomitant with the discussions regarding FIGS. 10 and 11 above, the impedance values within the cochlea at the first temporal location and the second temporal location are obtained using a cochlear implant electrode array implanted (or in the process of being implanted) in a human. In an embodiment, the first temporal location occurs during or shortly after a completion of a surgery implanting the electrode array into the cochlea, and on the same day as the surgery. In this regard, some embodiments include executing four-point impedance measurements before “closing” the incisions in the skin to access the implantation site. In some embodiments, four-point impedance measurements are taken shortly after closing the incisions in the skin, such as, for example, within 20 or 15 or 10 or 5 minutes of such. Indeed, in an exemplary embodiment, four-point impedance measurements are taken within 120, 90, 60 or 50 or 40 or 30 or 20 or 10 or 5 minutes, or any value or range of values therebetween in 1 minute increments of the electrode array first entering the cochlea and/or from a completion of a “packing” exercise where tissue or another substance is packed around the cochlear implant electrode array at the opening into the cochlea so as to establish a barrier. In an embodiment, the measurements are taken while the recipient is in the recovery room the day of the surgery. In some embodiments, the measurements are taken while the recipient is anesthetized, as the skin may be “sore” over the implant site (embodiments can use the external component of the cochlear implant, or the external component that interfaces with the implant detailed above, which will involve skin contact over the implant, and may not be as pleasant, hence some of the timeframes noted above). Indeed, the recipient may simply be irritable after coming out of anesthesia, and may not want anything (or anyone) touching his or her head for psychological reasons. Corollary to this is that in some embodiments, the first temporal location (and note that all of the aforementioned time frames are with respect to the first temporal location) may exist while the recipient is first sleeping after he or she comes out of the anesthesia. The first temporal location may simply be a location where the recipient will permit someone to place the external component against his or her head, at least without pain or with acceptable sensitivity. Thus, the first temporal location can exist, in some embodiments, within 1, 2, 3, 4, 5, 6, 7, 8, 9, 10, 11, 12, 13, 14, 15, or 16 hours of the electrode array first entering the cochlea, although embodiments should be practiced with the first temporal period being as close as reasonably possible to the first entry time.

But as seen above, there is a temporal correlation with the first temporal location and the implantation of the array in the cochlea. In at least some exemplary embodiments, the first temporal location is “same day” as the implantation of the electrode array in the cochlea. In at least some exemplary embodiments, the first temporal location should not extend into the second day after implantation (calendar day), but of course that can vary depending on the time of the surgery, where for example a surgery at 9 o'clock at night very well might warrant the first temporal location being on day two/the next day, hence the aforementioned temporal periods measured from the first entry of the array into the cochlea.

Thus, the first temporal location can exist, in some embodiments, within 1, 2, 3, 4, 5, 6, 7, 8, 9, 10, 11, 12, 13, 14, 15 or 16 hours of the electrode array first entering the cochlea, although embodiments should be practiced with the first temporal period being as close as reasonably possible to the first entry time.

But still, embodiments can include scenarios where the first temporal period is before or just after the surgery is completed, or at least before the patient is removed from the operating room and/or before the patient is moved to the more permanent recovery room (as opposed to an intermediate room where additional post operative actions might be taken without tying up a surgery room).

In an embodiment, the first temporal location can be between the final electrode array placement in the cochlea and exit from the operating room.

In an exemplary embodiment, the impedance values within the cochlea at the first temporal location and the second temporal location are obtained using a cochlear implant electrode array, the first temporal location is an intra-operative temporal location vis-à-vis implantation of the cochlear implant electrode array and the second temporal location is an intra-operative temporal location plus one day temporal location.

And note that there can be a temporal difference between the action of obtaining the data based on the impedance values and the taking of the impedance values. This is because method action 1210 can be executed by someone different or can occur after the action of actually implementing the four point impedance to obtain the actual impedance values upon which the data obtained in method action 1210 is based.

In an embodiment, the implemented treatment of method action 1230 is the execution of an optional cleaving of a prodrug previously provided to the cochlea. In an embodiment, the prodrug could have been placed into the cochlea (such as being co-located with the electrode array and thus implanted in the cochlea during insertion of the array) and then activated when method action 1230 is executed. Alternatively, there is a substance to cleave already in the cochlea (again, such as being co-located with the electrode array), and then the action of method action 1230 is to provide the prodrug systematically.

In an embodiment, the method 1200 further includes the method action of obtaining additional data based on impedance values within the cochlea at a third temporal location at least 2, 3, 4, 5, 6, 7, 8, 9, 10, 11, 12, 13, 14, 15, or 16 weeks or more after the second temporal location (such as by executing four point impedance with the electrode array implanted in the cochlea) and evaluating the obtained additional data and continuing, halting and/or adjusting the implanted treatment based on the evaluation of the additional data. In an embodiment, there is a decrease in the mean impedance values, such as by 3, 4, 5, 6, 7, 7, 8, 10, 11, 12, 13, 14, or 15%, or any value or range of values there between in 0.1% increments. In an embodiment, the method includes continuing and/or increasing a level of therapeutic treatment even though there is one of the decreases.

In an embodiment, the implemented treatment includes prescribing one or more of systemic steroids, anticoagulants, clot busters, antifibrotics, antiproliferatives or NSAIDs. As will be described in greater detail below, in at least some embodiments, the implant is configured to deliver a therapeutic substance to the inner ear of the recipient, and the implemented treatment of method action 1230 can alter the quantity of therapeutic substance that is delivered and/or the rate at which the therapeutic substance that is delivered. For example, the volume or concentration of a liquid therapeutic substance in the implant can be adjusted to control its rate of release by permeation or diffusion.

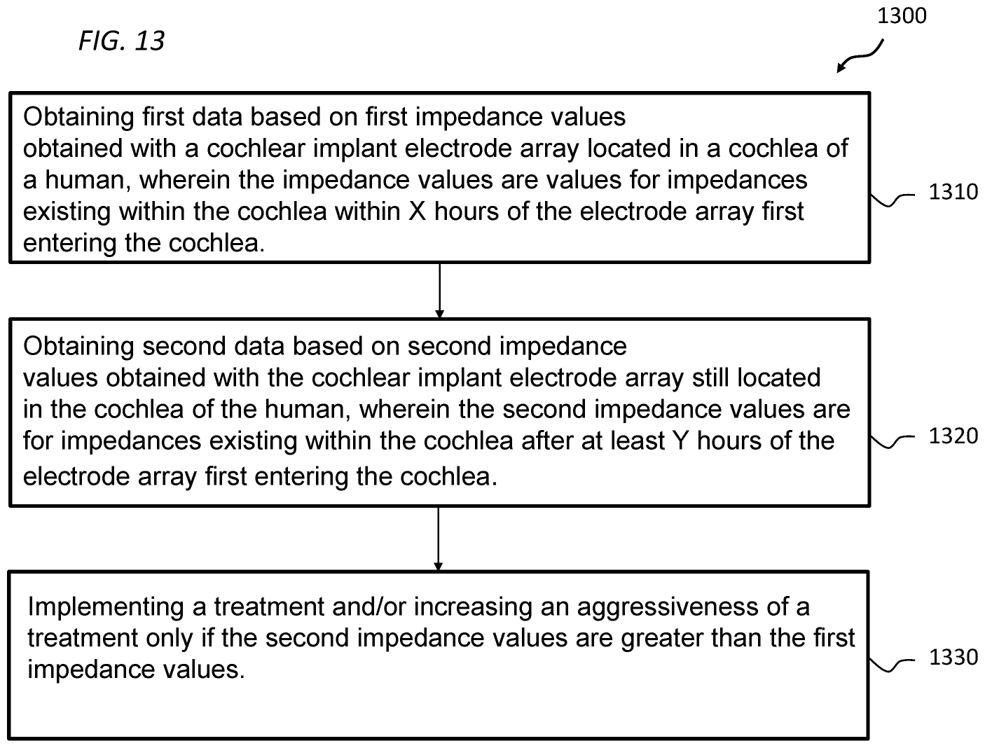

FIG. 13 shows another exemplary algorithm for an exemplary method, method 1300. Method 1300 includes method action 1310, which includes the action of obtaining first data based on first impedance values obtained with a cochlear implant electrode array located in a cochlea of a human, wherein the impedance values are values for impedances existing within the cochlea within X hours of the electrode array first entering the cochlea (or within X hours from final placement of the cochlea or within X hours from the end of the closure procedure, or within X hours from the time that the recipient leaves the operating room), wherein X can be 0.1, 0.2, 0.3, 0.4, 0.5, 0.6, 0.7, 0.8, 0.9, 1, 1.25, 1.5, 1.75, 2, 2.5, 3, 3.5, 4, 4.5, 5, 5.5, 6, 6.5, 7, 7.5, 8, 8.5, 9, 9.5, 10, 11, 12, 13, 14, 15, 16, 17, or 18 or more, or any value or range of values therebetween in 0.1 increments.

Method 1300 includes method action 1320, which includes obtaining second data based on second impedance values obtained with the cochlear implant electrode array still located in the cochlea of the human, wherein the second impedance values are for impedances existing within the cochlea after at least Y hours of the electrode array first entering the cochlea (or any of the other temporal markers noted above), wherein Y can be 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 or more, or any value or range of values therebetween in 0.1 increments. In an embodiment, Y does not exceed 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 41, 42, 43, 44, or 45 or more, or any value or range of values therebetween in 0.1 increments.

Method 1300 further includes method action 1330, which includes the action of implementing a treatment and/or increasing an aggressiveness of a treatment only if the second impedance values are greater than the first impedance values.

In an embodiment, the implemented treatment is a treatment that can have a deleterious effect on the human and the increase in the aggressiveness of the treatment can have a deleterious effect on the human. In an embodiment, the first and second impedance values obtained with the cochlear implant electrode array were obtained using four-point impedance techniques. In an embodiment, the first impedance values are impedance values that existed within 6, 5, 4, 3, 2, or 1 hours of the electrode array first entering the cochlea, concomitant with the teachings above, and the second impedance values are impedance values that existed more than 18 hours and less than 30 hours from the electrode array first entering the cochlea, again concomitant with the teachings above.

In an embodiment, the first and second impedance values obtained with the cochlear implant electrode array were primarily influenced by the environment surrounding and/or adjacent the electrodes used to measure the first and second impedance values, or otherwise the environment within the cochlea.

In an embodiment, the implemented treatment is a treatment for treating an ailment, the symptoms of which are not present at the time of implementing and the treatment the aggressiveness of which is increased is a treatment for treating an ailment the symptoms of which are not present at the time of increasing. In some embodiments, method 1300 further includes the action of normalizing the first data to account for variations in the impedance measurement that are statistically likely to dissipate with time.

As noted above, not all impedance values, or more accurately, not all read electrodes or the data therefrom need be used. In an embodiment, the first impedance values are impedance values for Z of the first 3, 4, 5, 6, 7, 8, 9, or 10, or any value or range of values therebetween in 1 increment read electrodes of the electrode array, the first of the number of electrodes being the most basal electrode, where Z can be 1, 2, 3, 4, 5, 6, 7, 8, 9, or 10, or any value or range of values therebetween, and can start 1, 2, 3, 4, or 5 read electrodes from the first. Moreover, the second impedance values are impedance values for any one or more or all of those electrodes (they need not be the same, and can have the same constraints just detailed). In some embodiments, the first impedance values are impedance values for Z of a first number of read electrodes of the electrode array, the first of the first number of electrodes being the most basal electrode, and the first number of read electrodes being located in the basal portion of the cochlea and the second impedance values are impedance values for Z of the first number of read electrodes or any one or more or all of the first number. And note that the second impedance values can include other read electrodes not used for the first (such as, for example, to make up for an extraneous datapoint or a failed electrode).

In some embodiments, values from one or more of the read electrodes can be discounted or otherwise disregarded, such as when, for example, the values appear to be extraneous or otherwise are anomalous. Indeed, as noted above, embodiments include normalizing or otherwise statistically adjusting the first data. The second data can also be normalized or otherwise statistically adjusted.

In some embodiments, any one or more of the following read electrodes, starting from the most basal read electrode (as oppose to the most basal electrode, which cannot be a read electrode), can be used to obtain the first and/or second impedances (and they need not be the same but can be the same): read electrode 1, 2, 3, 4, 5, 6, 7, 8,9, 10, 11, 12, 13, 14, 15, 16, 17, 18, 19, 20, 21, 22, 23, 24, 25, 26, 27, 28, 29, and/or 30 (the industry currently uses a maximum of 22 intra-cochlear electrodes total on most cochlear implant electrode arrays, but embodiments can use more extensive numbers of electrodes on arrays).

In an embodiment, the first impedance values are impedance values for all or a subset of a first number of read electrodes of the electrode array, the first of the first number of electrodes being the most basal electrode, and the first number of read electrodes subtending at least an angle of 110, 120, 130, 140, 150, 160, 170, 180, 190, 200, 210, 220, 230, 240, 250, 260, 270, 280, 290, 300, 310, 320 or 330 degrees or any value or range of values therebetween in 1 degree increments within the cochlea, and in some embodiments, not exceeding 170, 180, 190, 200, 210, 220, 230, 240, 250, 260, 270, 280, 290, 300, 310, 320, 330, 340, 350, or 360 degrees, or any value or range of values therebetween in 1 degree increments within the cochlea. In an embodiment, the second impedance values are impedance values for all or the subset or another subset of the first number of read electrodes.

In an embodiment, the most basal first, second, third, fourth, fifth, sixth, and/or seventh read electrode (e.g., read electrodes 1, 2, 3; 1, 2 and 5; or 1 and 6) are excluded from the electrodes used for the first and/or second values.

Scenarios can exist where impedance rises occur that are distinct from those resulting from the physiological change that causes the impedance increase 2, 3 or 4 months hence (which can be a result of an immune response), and can be a result of a broad response or otherwise caused by factors different from those upon which the teachings herein rely in some embodiments. For example, overt trauma that causes blood to enter the cochlea and can cause an impedance rise (locally or globally). Also, impedance risers in the cochlea could be a result of a holistic body response, such as an allergic reaction in the inner ear around the time of the implantation procedure. And the risers could be broad based, affecting the entire cochlea, or at least much of the cochlea, or at least much of the cochlea relative to the electrodes.

In view of this, embodiments include distinguishing from impedance values (including impedance increases) resulting from these other scenarios (or at least scenarios that are not a result of the physiological change in the cochlea detailed herein) and impedance values (including increases) that represent a latent variable for the physiological change that results in the impedance increase 2, 3 or 4 months hence.

In this regard, an analysis of the shape of an impedance curve and/or a curve representing an increase in the impedance, which can be developed according to the teachings herein, can include distinguishing an increase in impedance that forecasts the impedance increase in 2, 3, 4 or more months from an impedance increase that does not forecast the 2, 3, 4 month or more impedance increase. By way of example, statistical analysis techniques can be implemented on the data obtained during the operation/proximate the operation and/or on the data obtained after the operation on the following day (or whenever that occurs according to the teachings herein) to differentiate between the various scenarios causing impedance rise/changes, or at least identify an impedance rise that is used to forecast the subsequent impedance rise.

Corollary to the above, while many embodiments have focused on the change in impedance, embodiments can include focusing on characteristic shapes of an impedance increase curve that indicates/predicts the impedance increase 2, 3, or 4 months hence. By focusing on the curve shape (exclusively or non-exclusively), or otherwise the data distribution, a prediction can be made even if the increases are “diluted” or have components that correspond to impedance increases that are not related to the physiological change causing the downrange impedance increase.

Moreover, in some scenarios, trauma will be more likely to occur outside the basal region relative to locations in the basal region. Thus, there can be local blood releases that will increase the impedance at the read electrodes outside the basal region for example. This can skew the data.

And thus, in view of the above, embodiments can include focusing on the basal electrodes relative to other electrodes. Also, there can be local impedance rises that are due to factors different from those corresponding to the change in physiology in the cochlea (local bleeding proximate an electrode for example). These values can skew the average impedance increase, potentially causing the percentage increase to be above a threshold for treatment when treatment is not necessary or another treatment might be more suitable. Thus, embodiments that focus on the basal electrodes or otherwise compare the basal readings to the non-basal readings can be used to avoid “false positives.” Conversely, in an embodiment, the lack of the characteristic increase in impedance at the basal locations (relative to other locations) can be an indication that the other factors have caused the impedance increase. This too can be a catalyst for implementing one or more of the therapies, as the inability to evaluate the impedance increase can be sufficient justification to implement a therapy/increase a therapy that would otherwise not be implemented. In some embodiments, the entire increase curve will be shifted upwards by some amount (potentially linearly).

In an embodiment, the method 1300 further includes obtaining third data based on third impedance values obtained with the cochlear implant electrode array still located in the cochlea of the human, wherein the third impedance values for impedances existing within the cochlea for at least 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days, or any value or range of values therebetween in 1 day increments after the second impedance values existed, wherein the third impedance values are less than the second impedance values (such as less by any of the values noted above. The method further includes continuing implementation of the treatment and/or increasing the aggressiveness of the treatment for at least 30 days after obtaining the third data.

And again, it is noted that in some embodiments, data collection may occur from more than all of the electrodes that are utilized for the analysis. That is, the analysis may be limited to data from less than all of the electrodes from which impedance values were obtained. Any disclosure herein of a limitation with respect to the utilization of a read electrode with respect to collecting data also corresponds to an alternate disclosure of a limitation on the data that is utilized to perform the comparisons and/or analysis detailed herein where more data could be obtained than that utilized for the analysis/comparison.

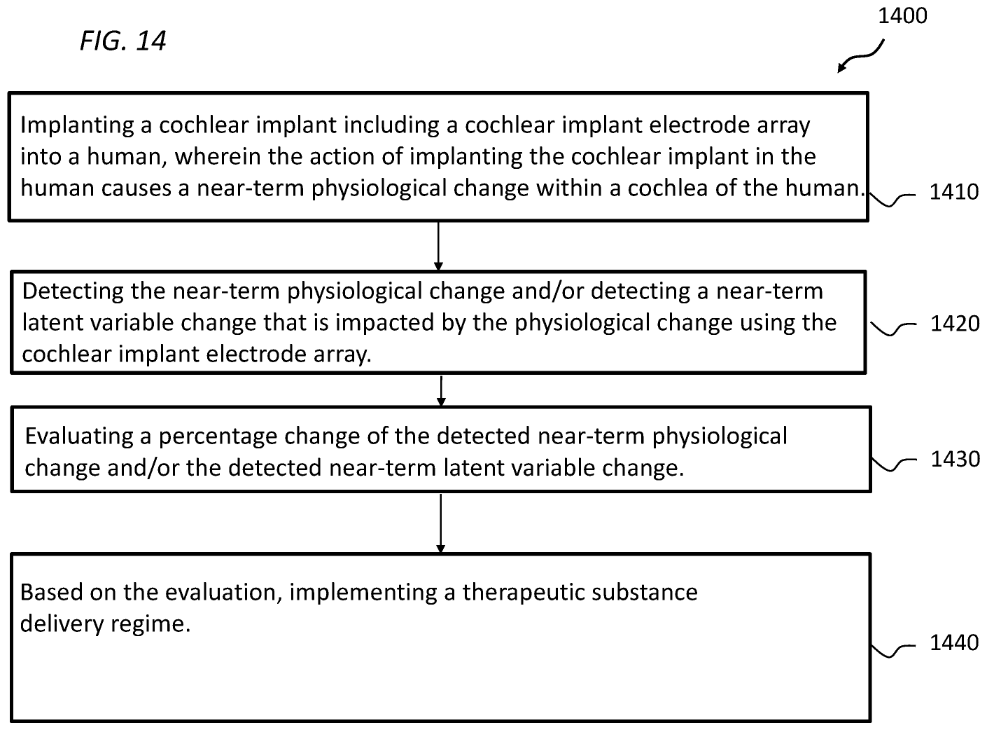

FIG. 14 shows an exemplary flowchart for an exemplary method, method 1400, that includes method action 1410, which includes implanting a cochlear implant including a cochlear implant electrode array into a human, wherein the action of implanting the cochlear implant in the human causes a near-term physiological change within a cochlea of the human. Some of these changes are described below. Method 1400 includes method action 1420, which includes detecting the near-term physiological change and/or detecting a near-term latent variable change that is impacted by the physiological change using the cochlear implant electrode array.

Method 1400 includes method action 1430, which includes evaluating a percentage change of the detected near-term physiological change or the detected near-term latent variable change, and method action 1440, which includes, based on the evaluation, implementing a therapeutic substance delivery regime.

In an embodiment, one of the detected changes is an increase in impedance within a duct of the cochlea in which the electrode array is located. This can be the impedance increase detailed above.

In an embodiment, the near-term physiological change occurs within 0.1, 0.2, 0.3, 0.4, 0.5, 0.6. 0.7, 0.8., 0.9, 1, 1.5, 2, 2.5, 3, 3.5, 4, 4.5, 5, 5.5, 6, 6.5, 7, 8, 9, 10, 11, 12, 13, 14, 15, 16, 17, 18, 19, 20, 21, 22, 23, or 24 hours, or any value or range of values therebetween in 0.1 hour increments of the implantation of the cochlear implant, or at least within first entry into the cochlea of the array and/or component used to access the cochlea (e.g., drill bit) and/or final setting of the array in the cochlea.

In an embodiment, the action of evaluating the percentage change includes comparing first data obtained during the surgery or effectively immediately thereafter to second data obtained after 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, or 38 hours, or any value or range of values therebetween in 0.1 hour increments after the first data was obtained and/or not exceeding 12, 13, 14, 15, 16, 17, 18, 19, 20, 21, 22, 23, 24, 25, 26, 27, 28, 29, 30, 31, 32, 33, 34, 35, 36, 37, 38, 39, or 40 hours, or any value or range of values therebetween in 0.1 hour increments. Some embodiments have the first data corresponding to the first temporal location above, and the second data corresponding to the second temporal location noted above.

In an embodiment, method 1400 further includes implanting a second cochlear implant including a second cochlear implant electrode array into a second human, wherein the action of implanting the second cochlear implant in the human causes a second near-term physiological change within a second cochlea of the second human, detecting the second near-term physiological change and/or a second near-term latent variable change using the second cochlear implant electrode array, evaluating a percentage change of the detected second near-term physiological change and/or the detected second near-term latent variable change and, based on the evaluation of the percentage change of at least one of the second changes, not implementing a therapeutic substance delivery regime, wherein the percentage change of the at least one second change is less than A %, and the percentage change of the near-term first change is more than A %, where A can be 5, 5.5, 6, 6.5, 7, 7.5, 8, 8.5, 9, 9.5, 10, 10.5, 11, 11.5, 12, 12.5, or 13, or any value or range of values therebetween in 0.1 increments. And note that method actions 1410, 1420, and 1430 can be repeated 2, 3, 4, 5, 6, 7, 8, 9, 10, 11, 12, 13, 14, 15, 16, 17, 18, 19, 20, 21, 22, 23, 24, or 25 times or more, or any value or range of values therebetween in 1 increment within 1, 2, 3, 4, 5, 6, 7, 8, 9, 10, 11, 12, 13, 14, 15, 16, 17, 18, 19, 20, 21, 22, 23, 24, or 25 days, or weeks, or months where at least and/or no more than 30, 35, 40, 45, 50, 55, 60, 65, 70, 75, or 80%, or any value or range of values therebetween in 1% increments (e.g., between 33.5 and 47%) of the times method action 1440 will be implemented, with any one of the values of A % noted above. The idea here is that for some people, the treatment will be implemented, and others not, depending on the A % values.

In an embodiment, the percentage change of the near-term change detected is at least an A % increase in a mean value of four-point impedance values for at least 2, 3, 4, 5, 6, 7, 8, 9, 10, 11, 12, 13, 14 or 15 measurement locations or any value or range of values therebetween in 1 increment along the electrode array.

In an embodiment, the therapeutic substance delivery regime is in addition to a dexamethasone treatment. In this regard, in at least some exemplary embodiments, the treatment can be a treatment that is already incorporated into a cochlear implant electrode array (embodiments include incorporating dexamethasone in an electrode array). For example, the therapeutic substance delivery regime can comprise increasing the rate at which dexamethasone is released into the cochlea.

Embodiments include a non-transitory computer readable medium having recorded thereon a computer program for executing at least a portion of a method, the computer program including code for statistically analyzing first data based on four-point impedance data obtained using an implanted cochlear implant electrode array in a cochlea during different temporal periods and code for outputting an indication regarding a state of the cochlea. By way of example only and not by way of limitation, the output could be a measure of trauma and/or disruption that can be used as a guide by a clinician. By way of example, the output could be a green or safe indicator for low touch, and orange or moderate indicator to indicate that the patient should be monitored, and/or a red or severe indicator indicating that intervention should be implemented. The code could instead or in addition be for outputting a warning.

It is noted that any disclosure herein of any method action corresponds to a disclosure of a computer readable medium having the code to execute that function provided that the art enables such, and vice versa. In an embodiment, the medium further includes code for automatically controlling an implantable component of a cochlear implant of which the electrode array is a part to execute four-point impedance testing to obtain the four-point impedance data. In an embodiment, the medium includes code for automatically implementing a therapeutic action based on the statistical analysis. This code can be in an implant, such as a totally implantable cochlear implant (and thus can execute one or more or all of the method actions herein capable of being implemented by the implant automatically). In an embodiment, the warning or indication is an increased likelihood of deleterious fibrosis in the cochlea. In an embodiment, the medium includes code for controlling an implantable component of a cochlear implant of with the electrode array is a part to release a therapeutic substance based on the statistical analysis.

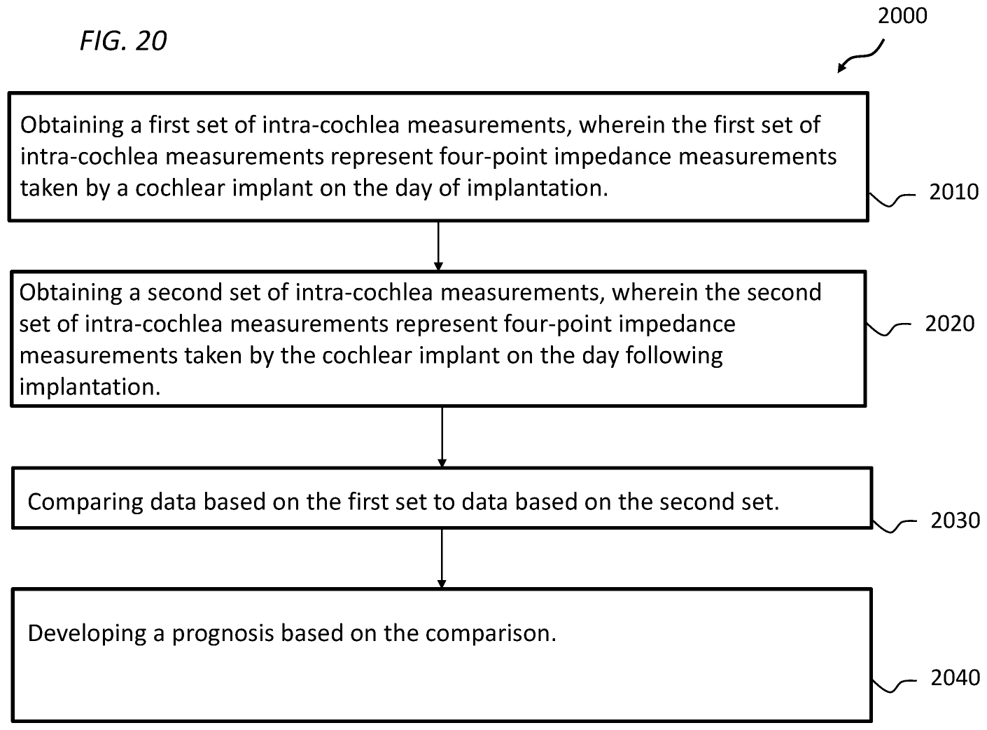

FIG. 20 presents an exemplary flowchart for an exemplary method, method 2000, which includes method action 2010, which includes the action of obtaining a first set of intra-cochlea measurements, wherein the first set of intra-cochlea measurements represents four-point impedance measurements taken by a cochlear implant on the day of implantation. In an embodiment, this can include executing the four-point measurements detailed above, or obtaining the measurements from a third party. The first set of measurements can be a set of all of the measurements obtainable from the electrode array, which in the embodiment of a 22 electrode cochlear implant electrode array, could be 19 measurement locations. But in an exemplary embodiment, this could be fewer, such as, for example, only the readings before and/or up to the basal turn. Thus, the first and/or second set of intra-cochlea measurements comprise an average impedance obtained from respective subsets of intra-cochlea electrodes of the cochlear implant, wherein the respective subsets of electrodes comprises electrodes implanted in the basal region of the recipient's cochlea up to (and including in some embodiments) the basal turn. That said, the first set and/or the second set could include all of the measurements, and as will be detailed below, only some of the measurements are utilized in the comparison. And in some embodiments, the first set and/or the second set of intra-cochlea measurements comprise an average impedance measurement obtained from all of the electrodes implanted within the recipient's cochlea.

Concomitant with the teachings above, method 2000 further includes method action 2020, which includes the action of obtaining a second set of intra-cochlea measurements, wherein the second set of intra-cochlea measurements represent four-point impedance measurements taken by the cochlear implant on the day following implantation. This set can have the same number of values as the first set, or can have a different number of values. With respect to a more pedestrian scenario where the values will be different, in some instances, one or more of the electrodes could fail after implantation. Or, more accurately, one or more of the leads to such electrodes could fail. In any event, it could be that there were fewer values in the second set of measurements. Alternatively, it may simply be utilitarian to obtain fewer values. Still, in some embodiments, the second set of measurements will be a complete set as was the case with the first. Any set of values that can enable the teachings detailed herein can be utilized in at least some exemplary embodiments. And while the teachings are directed towards obtaining sets of measurements, other embodiments could include sets of values based on measurements. For example, values could be normalized.

Method 2000 further includes method action 2030, which includes the action of comparing data based on the first set to data based on the second set. In an exemplary embodiment, method action 2030 can be a statistical comparison. By way of example only and not by way of limitation, all or some of the values of the first set of measurements (e.g., the measurements associated with locations prior to the first basal turn) can be averaged to obtain a mean and/or median or mode for that matter, the mode being within a statistical range. And this can be done for the values of the second set of measurements, all or some of them. And the number of values that are utilized from the second set need not be the same as the number of values utilized for the first. Again, in some embodiments, normalization and/or data vetting can be implemented. Extraneous data can be ignored or removed or otherwise at least smooth.

As seen, method 200 further includes the method action 2040, which includes the action of developing a prognosis based on the comparison. In an exemplary embodiment, this can include predicting whether or not there will be a meaningful and/or problematic and/or deleterious impedance in the cochlea one, two, three, four, five, six or more months, or any value or range of values taken in 1 week increments after the second set of measurements are taken (as distinguished from obtaining those measurements—again, the action of obtaining measurements can occur after the action of actually taking the measurements). In an embodiment, the prognosis includes predicting whether adjustments of a significant manner will be likely to be needed to the output current of the cochlear implant (to account for the increase in the impedance). In an exemplary embodiment, this can include predicting whether or not there will be fibrous tissue growth, or otherwise whether there is a statistical likelihood that there will be meaningful fibrous tissue growth, within the cochlea. This fibrous tissue growth could be significant fibrous tissue growth. In an embodiment, the fibrous tissue growth could be tissue growth that creates an impedance within the cochlea that can have deleterious effects relative to that which would otherwise be the case. In some embodiments, fibrous tissue can impinge on structures within the cochlea and have an adverse impact on residual hearing. For example, fibrous tissue can impinge on the basilar membrane and interfere with natural perception of low frequency sound. Treatments herein can reduce and/or eliminate this fibrous tissue growth.

In an embodiment, the prognosis is that there will be a statistically significant chance that there will be a physical phenomenon within the cochlea, at one or more of the locations in the future detailed herein, such as two or three or four months hence, that will result in an impedance within the cochlea with respect to utilization of the electrodes that would be, on average, at least in certain portions of the cochlea, such as, for example, the locations before the basal turn or at least some of the locations before the basal turn, cause the electrode array to have to output current at higher current levels, including significantly higher current levels, to meet a threshold level and/or a comfort level relative to that which would otherwise be the case in the absence of this physical phenomenon. In an exemplary embodiment, there is a prediction that the impedance will increase on average (mean, median, and/or mode) by at least 15, 20, 25, 30, 35, 40, 45, 50, 55, 60, 70, 80, 90, 100, 125, 150, 175, or 200% or more, or any value or range of values therebetween in 1% increments within 2, 3, 4 or 5 months.

Thus, in an embodiment, the prognosis is a prognosis regarding a future efficacy of the cochlear implant sometime after and/or before and/or at 5, 6, 7, 8, 9, 10, 11, 12, 13, 14, 15, 16, 17, 18, 19, or 20 or more, or any value or range of values therebetween in 1 day increments weeks after the first set is obtained, to accurately reproduce sounds, where this accuracy is impacted at least in part by increase in impedance in the cochlea relative to that which would be the case if, for example, there was no increase in fibrous tissue growth in the cochlea. Embodiments include scenarios where the prognosis is a prognosis regarding electrical impedance and/or fibrous tissue growth within the cochlea sometime after and/or before and/or at any one or more of the aforementioned values. And it is noted that in at least some scenarios, there always be some form of fibrous tissue growth within the cochlea. And this can impact the impedance. But the teachings detailed herein detect or otherwise protect scenarios where there will be meaningful fibrous tissue growth that will impact the performance on the cochlear implant, such as, for example, requiring the cochlear implant to have an output current that is higher relative to that which would otherwise be the case to evoke a same level of volume with respect to the hearing percept evoked. The greater the impedance, the more current needs to be outputted by the electrodes to achieve the same volume, all other things being equal.

In an embodiment, method 2000 further includes the action of recommending an intervention based on the comparison. In an exemplary embodiment, method 2000 further includes the action of recommending an intervention when the comparison represent an impedance increase of A % or more compared to the first set of intra-cochlea measurements. In an embodiment, the second set of intra-cochlea measurements comprise an average impedance obtained from a subset of intra-cochlea electrodes of the cochlear implant, wherein the subset of electrodes comprises electrodes implanted in the basal region of the recipient's cochlea up to the basal turn. In some embodiments, method 2000 includes recommending an intervention when the comparison reveals that the second set of intra-cochlea measurements represent an impedance increase of 5, 6, or 7% or more compared to the first set of intra-cochlea measurements (again, based on an average of the impedance measurements for all or some of the electrodes).

Embodiments include controlling the cochlear implant based on the prognosis. Embodiments include adjusting settings that will be utilized to control the cochlear implant based on the prognosis. For example, the compliance voltage of one or more stimulation current sources/channels within the cochlear implant can be adjusted based on the comparison executed in method action 2040 to ensure or otherwise provide or increases the chances that adequate electrical stimulus is delivered to the recipient. Typically, this will be an increase in the output current relative to that which would otherwise be the case. By way of example only and not by way of limitation, cochlear implants include map data that is stored in the cochlear implant, which map data is utilized to control outputs of the electrodes based on received input based on ambient sound captured by a microphone or otherwise a sound capture device of the cochlear implant. A given map will result in an output that is perceived by the recipient that is different from another map, and sometimes significantly different from another map, all for the same input. Embodiments thus include utilizing the comparison executed in method action 2030, and/or the prognosis executed in method action 2040, to adjust control of the cochlear implant or otherwise select maps that will be used for the cochlear implant relative to other maps, or otherwise create maps that would be different relative to other maps that would be used in the absence of the prognosis and/or the comparison. In some embodiments, these maps can be significantly different and, in some embodiments, a given map selected or otherwise recommended could be a map that is very unpleasing or otherwise undesirable in a cochlea that does not experience the predicted impedance rises or various physiological changes detailed herein.

In some embodiments, the aforementioned developed map or otherwise selected map could be a map that is implemented at a time period after the first and/or second measurements are taken. For example, in an embodiment, a first map could be utilized for the first month or so, or at least the first month of utilization of the cochlear implant, during a time period where, for example, impedance values are below that which would be predicted at the time periods hence (e.g., three months after implantation). This first map could operate at lower current levels relative to the second map. These lower current levels could be significantly lower. Then, after a period of time of utilizing the first map, the second map could be implemented, which would supersede some or all of the settings of the first map. The second map could have a higher current level output for a given input relative to the first map, at least for some channels, such as, by way of example, the channels associated with the electrodes located before the first basal turn. In an embodiment, the second map could be implemented automatically, either after a set period of time, or upon a determination that impedance is beginning to rise and/or has risen within the cochlea. And in this regard, it is noted that impedance measurements are not limited to the time frames of within a day or two or three of implantation. Embodiments can include periodically and/or continuously testing the impedance within the cochlea. A utilitarian feature here is that the expected impedance rise or otherwise the impedance rise that ultimately occurs can be predicted, and thus plans for such can be implemented. One exemplary plan is the existence of a pre-positioned or a pre-created map that can be utilized when and/or after the impedance rise occurs, this map being the aforementioned second map. Another exemplary plan involves automated adjustment to the compliance voltage of the implant to accommodate expected physiological changes within the cochlea.

In some embodiments, the implementation of the map can occur automatically upon an indication of an impedance rise of a certain amount, where, for example, the cochlear implant can autonomously and/or under the control of a remote clinician, measure impedance within the cochlea. In an embodiment, impedance measurements are recorded (or obtained) every day or every 12 hours or some utilitarian period, and these impedance measurements could be evaluated (automatically) by the cochlear implant with the external component or a remote component, or could be sent to a remote location for evaluation every day or every week or some other utilitarian period. When the impedance begins to rise at least to a certain level, or a sufficiently high gradient is seen, a remote clinician or the like can implement the second map, or, for example, the map could be automatically implemented in embodiments where the cochlear implant or a component that is in the possession of the recipient evaluates the impedance rise. Accordingly, embodiments include coupling the prediction of an alternate increase in impedance with continuous monitoring otherwise sufficiently efficacious monitoring of impedance within the cochlea and correlating the impedance rise measurements with maps that are stored in the cochlear implant or otherwise can be uploaded to the cochlear implant or otherwise the adjustment of settings of the cochlear implant to accommodate the impedance rise, and these actions can be executed automatically because of the initial production. This as compared to a scenario where these impedance rises are seen, but they are unexpected or otherwise occur in a manner that was not predicted. That is, the predictive qualities of the teachings detailed herein permit proactive development of maps and the more rapid implementation of such relative to that which would otherwise be the case. By way of example only and not by way of limitation, in an exemplary embodiment, settings or otherwise new maps can be implemented within 1, 2, 3, 4, 5, 6, 7, 8, 9, 10, 11, 12 hours, or any value or range of values therebetween in one minute increments of the subsequent increase in impedance that is predicted (as distinguished from the first and second day impedance differences). This can thus provide a utilitarian reaction time so that the recipient will have a more satisfactory experience or at least a utilitarian experience with his or her cochlear implant even though the impedance is increasing, relative to that which would otherwise be the case in the absence of the protections provided by the teachings detailed herein.

Embodiments include transition maps. The impedance will not rise from one level to the predicted level overnight for example or within hours or minutes or probably not even days. The rise in impedance will occur more gradually, and thus there can be a map that transitions from the first map to the second map, thus moving the increase in output current for a given stimulus for example. In some embodiments, the transition map is not necessarily a map at all, but instead an algorithm that increases or otherwise makes adjustments to the first map to gradually increase values for example, on a temporal basis alone or and/or on a basis that is correlated to the impedance values read within the cochlea, or instead decreases the current levels for the second map, which decreases are steadily or periodically or utilitarian way reduced over time to accommodate the increase in impedance within the cochlea.

Embodiments can include the development of more than two maps, such as three or four or five or six or more maps, where the respective maps are implemented over time and/or based on the impedance rise to accommodate the impedance rise and ultimately condition the recipient for use of the higher, more aggressive output maps, when the predicted increase in impedance to the predicted impedance levels or to relatively high impedance levels come to pass.

Embodiments also include delaying the so-called “switch-on” of the cochlear implant relative to that which would otherwise be the case for standard practice or statistical based practice. By way of example only and not by way of limitation, if the comparison indicates the aforementioned rise in impedance, with the prognosis indicates that there will ultimately be a large amount of fibrous tissue growth within the cochlea, the switch-one date of the cochlear implant could be delayed by at least, for example, 10, 15, 20, 25, 30, 35, 40, 45, or 50 days or more, or any value or range of values therebetween in one day increments. In an exemplary embodiment, this could have utilitarian value with respect to preventing extra tissue growth that could occur owing to the activation of the cochlear implant electrode array, where the electrical current could, conceivably, induce the fibrous tissue growth that could increase the impedance in the cochlea at the two or three or four month time period. Conversely, in an exemplary embodiment, there could be the action of applying relatively high current to tissue utilizing at least some of the electrodes so that the current will potentially prevent the fibrous tissue growth at those locations relative to that which would otherwise be the case.

Embodiments thus can include as part of the therapy a ramp up use of electrodes based on the impedance increase. For example, a soft start on electrodes where there is seen an increase in the impedance can be implemented. This can allow the healing process more time to happen, at least in the region of high four point impedance.

In some instances, trickle current and/or a switch on can be later for those with the higher impedance increases noted above. And in some embodiments, the application of white noise for example, can be used as part of some therapies.

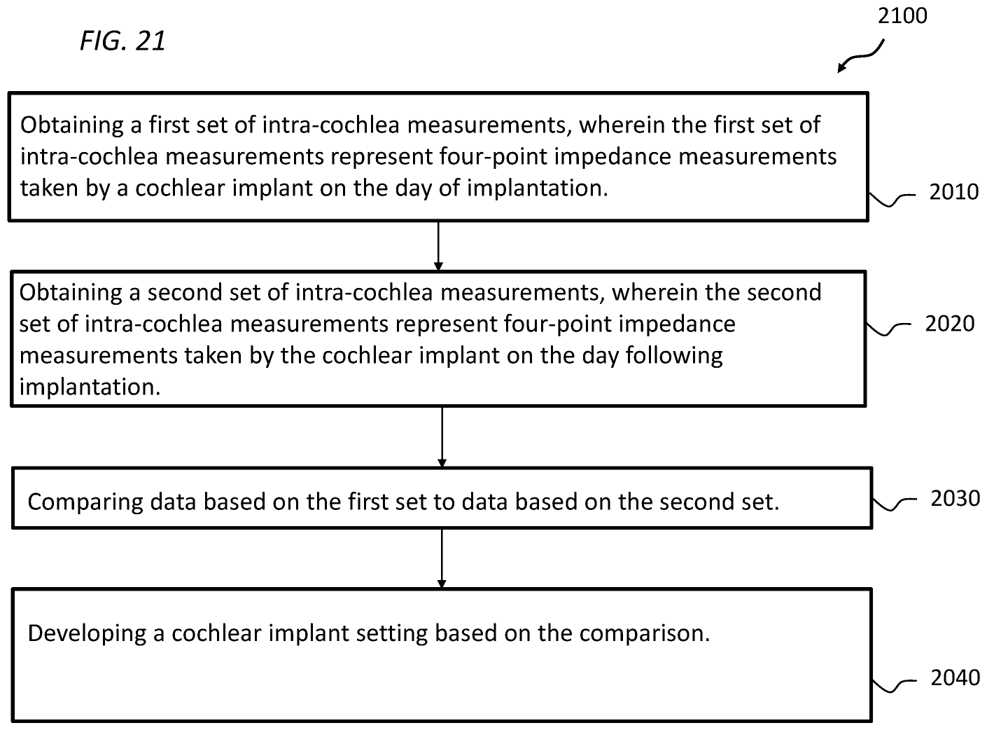

FIG. 21 presents an exemplary method, method 2100, according to an exemplary embodiment. Method 2100 includes method 2010, 2020, and 2030, which are detailed above. Method 2100 further includes method action 2040, which includes developing a setting for the cochlear implant based on the comparison. Concomitant with the teachings detailed above vis-à-vis developing a map for the cochlear implant the setting could be in output level for a given channel of a cochlear implant. The output level could be a threshold level and/or a comfort level. And consistent with any fitting and/or map development detailed herein, the fitting and/or map development or otherwise the setting is customized to the particular recipient, at least in some exemplary embodiments. By way of example only and not by way of limitation, the setting could be a threshold and/or comfort level setting for any one or more of the channels the cochlear implant. Concomitant with the teachings detailed herein, in at least some exemplary embodiments, the setting could be a setting for the channels associated with the electrodes located before the first basal turn. The settings could be for a subset of those electrodes, or could be for all or almost all of the electrodes or otherwise the channels of the cochlear implant. The key here is that in at least some exemplary embodiments, the cochlear implant settings are developed based on the comparison otherwise the prognosis in other embodiments, of what is going to happen within the cochlea a number of months after the second set of data has been taken utilizing four-point impedance.

Corollary to this is that in at least some exemplary embodiments, method 2100 includes the action of determining a fitting profile for the cochlear implant from a comparison of the first set of intra-cochlea measurements with the second set of intra-cochlea measurements, wherein in at least some embodiments, the setting is a setting utilized in that fitting profile that is developed. In an embodiment, the fitting profile can be a map for example or some other set of data that can be utilized by a cochlear implant. This does not mean that the action of fitting occurs, although it can. This simply means that a fitting profile is developed which can be utilized to actually fit the cochlear implant. But of course, in some embodiments, there includes the action of fitting the cochlear implant utilizing the determined fitting profile. And, in some embodiments, there is the action of fitting the cochlear implant based on the comparison, where the determined fitting profile was not done by the same person that is actually fitting the cochlear implant.

In some embodiments, the fitting profile that is developed is a fitting profile for statistically high impedance profiles within the cochlea whereas in other embodiments, or more accurately, in other scenarios, the fitting profile is a fitting profile for statistically normal impedance profiles within the cochlea. By way of example only and not by way of limitation, with respect to the former, if the comparison indicates otherwise predicts that there will be any increase in impedance that is significant in accordance with the teachings detailed above, or otherwise herein, this impedance increase contended be an increase that is statistically high or otherwise corresponds to impedance values that are statistically high based on a similarly situated population demographically. By way of example only and not by way of limitation, the person at issue can be a human factors engineering 10% to 90% male and/or female or any value or range of values therebetween in 1% increments, born in the United States of America, the European Union, United Kingdom, the Republic of France, the Federal Republic of Germany, Japan, Australia, New Zealand, or the People's Republic of China, and a citizen thereof, having lived the majority of his or her life in those jurisdictions, being born at any date or range of dates from Jan. 16, 1930 to May 1, 2022, or otherwise being an age of 1 to 100 years old, and any increment therein in 1 year increments. Thus, the statistical significant values can be compared for any one or more of these human factors engineering groups.

In an exemplary embodiment, the statistical differences can be 0.25, 0.5, 0.75, 1, 1.25, 1.5, 1.75, or 2 or more standard deviations from the average for a given human factors engineering group or any value or range of values therebetween in 0.05 increments.

Embodiments include determining a first map for the cochlear implant from the comparison of the first set of intra-cochlea measurements with the second set of intra-cochlea measurements, wherein the first map is a default map for switch-on of the cochlear implant. This was detailed above. Embodiments can include determining a second map for the cochlear implant from the comparison of the first set of intra-cochlea measurements with the second set of intra-cochlea measurements, wherein the second map is a default map for the cochlear implant at a period of about 4, 5, 6, 7, 8, 9, 10, 11,12, 13, 14, or 15 weeks, or any value or range of values therebetween in 1 week increments from implantation.

Embodiments include determining a transition profile from the first map to the second map. This can be based on the comparison and/or the development of the first map and the second map.

As noted above, embodiments include comparing monitoring four-point impedance changes following cochlear implantation. In some embodiments, the four-point impedance monitoring provides information regarding the bulk biological environment surrounding the electrode array which is not discernible with conventional impedances. Indeed, testing in adult cochlear implant recipients with no measurable hearing prior to implantation and implanted with a perimodiolar cochlear implants show that such monitoring can have utilitarian value as noted above. In this regard, testing was implemented to validate some of the concepts detailed above, and will now be briefly described. It is noted that any disclosure herein of the testing actions and/or features and/or occurrences associated with the following details of the testing corresponds to a disclosure of a method action herein and/or a parameter of a method action or associated with a method action. Any device and/or system used to implement the testing detailed below corresponds to a disclosure of such.

In this testing, mean values for four-point and common ground impedances were calculated for all electrode contacts at intra-operative, 1-day, 1-week, 4/6-weeks and 3-months post-implantation. Linear mixed models were applied to the impedance data to compare between impedances and time points. Furthermore, patients were divided into groups dependent on the normalized change in four-point impedance from intra-operative to 1-day post. The normalized change was then calculated for all other time points and compared across the two groups. In this testing, significant increases in four-point impedance occurred 1-day and 3-months following surgery, particularly in the basal half of the array. Four-point impedance at 1-day was highly predictive of four-point impedance at 3-months. Four-point impedance at the other time points showed marginal or no increases from intra-operative. Patients with an average increase higher than 10% in four-point impedance from intra-operative to 1-day, had significantly higher values at 3-months (p=0.012). These patterns were not observed in common ground impedance. It was found that increases in four-point impedance within 24 hours of cochlear implantation had a predictive value for increases in impedance 3 months in the future.

It is briefly noted that while the following focuses mostly on method actions, devices, and/or systems that can implement these methods, at least some of the method actions of the methods, or included in some embodiments. In an exemplary embodiment, there is a computing device, such as a laptop and/or desktop computer, or the device(s) detailed above, that is programed or configured to execute one or more of the method actions detailed herein. Moreover, the teachings detailed herein can be executed utilizing remote processing. By way of example only and not by way of limitation, a remote server remote from a surgical location for example or otherwise remote from the cochlear implant electrode array that is being utilized to implement at least some of the method actions detailed herein or otherwise utilized to obtain the impedance values detailed herein can be utilized to execute any one or more of the method actions herein provided that the art enables such. In an embodiment, there is a chip and/or a processor configured to implement at least some of the method actions herein. There are also embodiments that include a system, such as the device 10 of FIG. 1A and/or one or more of the components of FIG. 7, that includes for example an implantable portion of a cochlear implant electrode array including back telemetry circuity and a plurality of electrodes in signal communication directly or indirectly with the back telemetry circuitry and an external device configured to be in signal communication with the implantable portion and configured to receive data from the implantable portion via the back telemetry circuity (the external device can be the behind-the-ear device and/or the external component and/or any of the control components or control units or external devices detailed herein. And it is noted that in some embodiments, the system need not include the implantable component whereas instead, the system can be configured to receive data based on four-point impedance measurements from whatever source, whether that be from an implantable component, or from a data distribution device that provides the information to the system. Indeed, in an exemplary embodiment, the data based on the four-point impedance measurements can be typed in by a human to the system. In any event, in an exemplary embodiment, the implantable component is configured to implement four point impedance measurements and provide the results of the measurements to the external device via the back telemetry circuity, and the system includes circuitry and/or software configured to evaluate the results and provide an indication that there is an impedance increase relative to a prior measurement that is predictive of a future deleterious development within a cochlea in which the electrode array is implanted. In an exemplary embodiment, the system is configured to execute one or more of the method actions detailed herein providing that the art enables such. Indeed, in an exemplary embodiment, the system includes artificial intelligence capabilities and/or is a trained neural network system and/or is an expert system that is configured to analyze the data and execute one or more of the method actions detailed herein.

Embodiments include methods and systems to prescribe further treatment locally (e.g., some controlled release system in implant, or middle ear intervention by way of example only) owing to the identified rise in impedance over the daylong interval from implantation to the second set of measurements, or any of the other temporal periods detailed herein indicated is potentially have utilitarian value with respect to predicting an ultimate rise in impedance in the out months. Embodiments include methods and systems to prescribe further treatment systemically with justified risk (e.g., systemic steroids, anticoagulants, clot busters, antifibrotics, antiproliferatives (anti NBF for example) NSAID's etc.), which may not be treatments one would implement without additional evaluation (which evaluation can correspond to those detailed herein). Embodiments can provide an objective measure of effectiveness of medical intervention. Embodiments can provide a biomarker for insertion atraumaticity. Embodiments can include automatic measures taken on day 2 with a BTE or the implant or the devices detailed above. Subsequent measures can be done remotely. Moreover, embodiments can include the application of a prodrug provided to the ear, where, in some embodiments, the method includes only cleaving the prodrug if four point impedance increase is seen. Thus, in at least some exemplary embodiments, the methods include providing one or more therapeutic substances in the cochlea at the time of implantation, such as in a manner that is co-located with the cochlear implant electrode array, such as, for example, in a well of the electrode array and/or as part of a substance that is entrained with the carrier member of the electrode array. In some embodiments, the cleaving agent could be taken systemically, or in any manner that can have utilitarian value, etc.

In some embodiments, the therapeutic substance can be provided as part of a local reservoir that can be controllably opened to the cochlea but otherwise remains sealed to the perilymph or otherwise protected against interaction with the perilymph, at least with respect to short-term periods. In some embodiments the material could diffuse over time, but the diffusion could be sufficiently low so that the effect of the substance is de minimus—this as compared to releasing the therapeutic substance in a manner that will have efficacy/releasing the therapeutic substance over a short enough time period that the therapeutic substance will have an impact on the recipient, which impact can in at least some exemplary scenarios, be problematic even though there are other utilitarian aspects of the release, this problematic scenario being one of the reasons, for example, why the therapeutic substance is not implemented or otherwise utilized as a matter of course). If, for example, the therapeutic substance could be released from the reservoir in a manner that will have efficacy. Otherwise, the therapeutic substance will be maintained in the reservoir and otherwise not released, or released over a period of time in sufficiently minimal increments so that the deleterious effects associated with the therapeutic substances are minimize and/or otherwise will not occur. This as a way to eliminate the therapeutic substance in a manner that avoids an accidental release at some point over the next 10 or 20 years. Alternatively, and/or in addition to this, a negating substance can be applied which will negate the effects of the therapeutic substance or otherwise deactivate the therapeutic substance were otherwise neutralize the therapeutic substance this can be done upon the completion of the comparison between the first set of data and the second set of data where any impedance increase is shown to be below any of the thresholds detailed herein.

In at least some exemplary embodiments, the action of deactivating or otherwise negating the therapeutic substance that is originally located in the cochlea can be executed within one or two or three or four or five days and/or weeks and/or months of the comparison. In an exemplary embodiment, this action can be executed after the third or fourth month after implantation whereafter of sufficient period of time to ensure that indeed there is no increase in the impedance values, or more specifically, no significant increase in impedance values over the timeframe of concern.

Accordingly, embodiments can include the utilization of an implant that includes a reservoir of therapeutic substance. This therapeutic substance could be activated or otherwise applied upon the prediction that there will be the fibrous tissue growth or otherwise upon the increase in the impedances detailed herein. Otherwise, the therapeutic substance would not be utilized. In an exemplary embodiment, the therapeutic substance could diffuse over a very long period of time and thus effectively have little to no negative consequences to the recipient relative to that which might be the case if the therapeutic substance was utilized as part of a treatment regime. In at least some embodiments, the rate that a therapeutic substance is released into the cochlea and/or the quantity of therapeutic substance released into the cochlea can be increased from a baseline release rate or quantity in response to an increase in the impedances detailed herein.

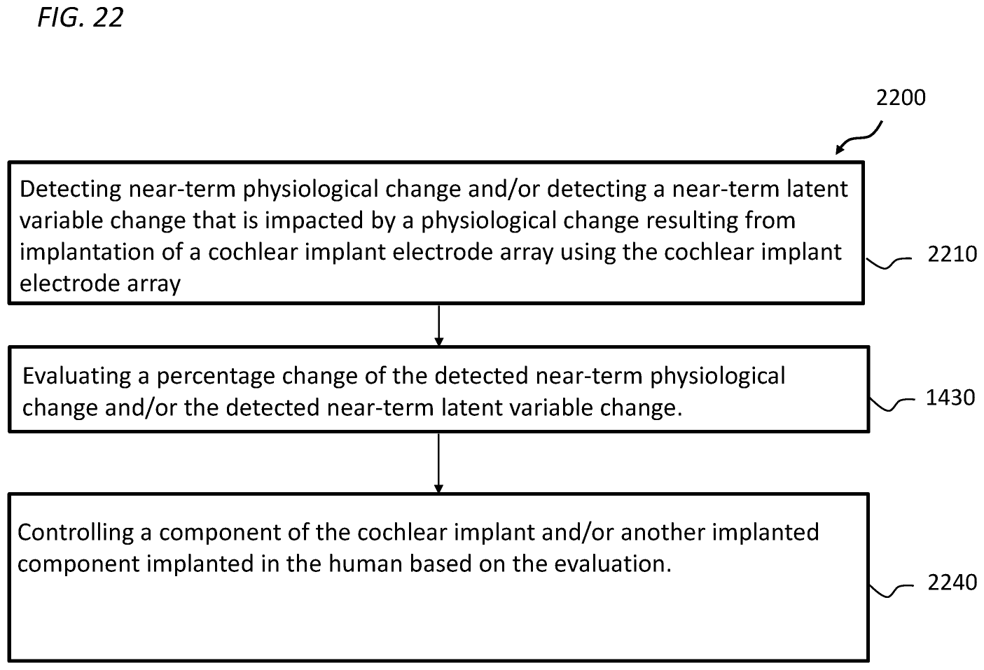

Thus, with reference to FIG. 22, in an embodiment, there is a method, method 2200, which includes various method actions, wherein in this exemplary embodiment, the method includes method action 2210, which includes detecting a near-term physiological change resulting from implantation of a cochlear implant electrode array in a cochlea and/or detecting a near-term latent variable change that is impacted by a physiological change resulting from the implantation of the cochlear implant electrode array using the cochlear implanted electrode array. This can be executed utilizing four-point impedance, or any other detection arrangement that can have utilitarian value. Method 2200 further includes method action 1430, which is detailed above, and which will not be repeated here for purposes of textual economy. (The method can also include method action 1410.) But as seen, method 2200 further includes method action 2240, which includes the action of controlling a component of the cochlear implant and/or another implanted component implanted in the human based on the evaluation.

In an exemplary embodiment, with respect to the action of controlling the cochlear implant, this can include the output of current at a level above that which would be the case for a given input in the absence of the control based on the evaluation. With reference to the above examples of utilizing different maps and/or utilizing different current levels for threshold and/or comfort levels, if the evaluation indicates the predicted or otherwise the production of an increase in impedance in the cochlea two or three or four months out, a different maps can be utilized or the current level of the output current can be increased relative to that which would otherwise be the case. There can be delayed activation time or start time of use of the cochlear implant mentioned above. And, in some embodiments, there can be the release of a therapeutic substance from the cochlear implant based on the evaluation. With respect to this latter aspect, in embodiments where there is a reservoir for example, a therapeutic substance in the cochlear implant or otherwise part of the cochlear implant rather attached to the cochlear implant, that was implanted with the cochlear implant, the cochlear implant can be controlled to release the therapeutic substance.

Accordingly, in an exemplary embodiment, the action of controlling the cochlear implant executes an implementation of a therapeutic substance delivery regime. In an exemplary embodiment, the action of controlling the cochlear implant releases a therapeutic substance from the cochlear implanted in the human at the time that the cochlear implant was implanted. This can be consistent with the concept of providing a therapeutic substance with the cochlear implant as a precaution or otherwise pre-positioning the therapeutic substance so that reentry into the cochlea after the implantation procedure need not occur. Thus, embodiments include controlling the cochlear implant to deliver a therapeutic substance without having to execute an additional surgical procedure after the day of implantation or after the end of the implantation process otherwise after the patient leaves the operating room. And, consistent with at least some exemplary embodiments, the action of controlling the cochlear implant provides a therapy to the human to limit future impedance increase within the cochlea, were limit also covers eliminate otherwise completely prevent. In an exemplary embodiment, the future impedance increase can be any of those detailed herein which are predicted to occur within one or two or three or four or more months after the day that the cochlear implant is implanted.

Embodiments include scenarios where the action of controlling the cochlear implant controls a mechanical component of the cochlear implant to release a chemical from the cochlear implant, which chemical would not be released in a short-term period in the absence of an evaluation of the percentage change being above or at a certain level. In an exemplary embodiment, this can entail the action of opening a valve to release a therapeutic substance in the form of a fluid located in the reservoir into the cochlea where, for example, the reservoir could be located in the cochlea or could be located in the middle ear, and there can be a conduit from the reservoir into the cochlea that is part of the electrode array, where an outlet of the conduit is located in the cochlea. The valve can be located in the cochlea, or upstream of the outlet. In some embodiments, these actions of controlling the cochlear implant are executed automatically, while in other embodiments, there executed under the control of a clinician or the like. In an exemplary embodiment, the cochlear implant could be any one or more of the devices or have any one or more of the features disclosed in U.S. Patent Application Publication No. 2021 0308371 to Daniel Smyth, published on Oct. 7, 2021, and/or U.S. Patent Application Publication No. 2022/0032020 to Daniel Smyth, published on Feb. 3, 2022, all by way of example and not by way of limitation. Still with reference to FIG. 22, method action 2240 includes as an alternative action, controlling another implanted component implanted in the human based on the evaluation. In an exemplary embodiment, a port prosthesis or the like is implanted during the procedure of implantation of the cochlear implant, or more accurately, during the time period where the access to the middle ear or otherwise the outer wall of the cochlea exists that enabled the cochlear implant electrode array to be inserted into the cochlea. In at least some exemplary embodiments, a second cochleostomy potentially (assuming that there is a first cochleostomy for the electrode array) or otherwise a second access location is obtained to access the interior of the cochlea. A device that can be dedicated to the action of providing a therapeutic substance to the cochlea can be implanted or otherwise attached to the cochlea so that the therapeutic substance can be provided to the cochlea at a later point in time. Accordingly, embodiments include the action of implanting a second implant that can execute one or more of the functionalities detailed herein, which second implant is separate from the cochlear implant.

In some embodiments, there can be a polymer on the surface of the electrode array, such as the carrier member, that is triggered by some form of stimulus, such as by light or by certain RF or sound frequencies, etc., in the action of controlling the implant. Still further, in some embodiments, a substance can be injected into the cochlea, or a substance can be ingested by the person, to result in an activation or a triggering of a substance that is carried otherwise as part of the cochlear implant to implement the therapy.

In some exemplary embodiments, the timeframe to begin the treatment occurs within one, two, three, four, five, six, seven, or eight days, or any value or range of values therebetween in one day increments after the second temporal location, and, as noted above and herein, this therapy can begin before there is any noticeable fibrous tissue growth within the cochlea.

In an exemplary embodiment, the second implant is a dedicated therapeutic substance delivery device or is a dedicated therapeutic substance enabling delivery device. The second implant can include a reservoir and/or a valve that can be controlled by the cochlear implant or by another component or by an external signal for example. By controlling this implant, the therapeutic substance can be metered into the cochlea if the analysis or otherwise the action of evaluating the percentage changes indicate utilitarian value in doing so, at least where the utilitarian value overcomes one or more or all deleterious effects associated with providing the therapeutic substance. (And in an exemplary embodiment, this second implant can include a relief valve which can, for example, release the therapeutic substance into the middle ear, in one fell swoop or over time, as opposed to releasing the therapeutic substance into the cochlea, as a way to “dispose” of the therapeutic substance upon a determination that the therapeutic substance is not needed or otherwise should not be provided.)

Any disclosure herein of an action associated with the cochlear implant electrode array vis-à-vis implementing a therapy or the like corresponds to an alternate disclosure of utilizing this alternate or otherwise the another implant for doing so, in the interest of textual economy, providing the art enables such unless otherwise noted. In an exemplary embodiment, the another implant is an implant that does not have electrical cochlear stimulation capabilities (but again, could be controlled by the cochlear implant or otherwise coupled to or in signal communication therewith).

In an exemplary embodiment, the implant could be any one or more of the devices or have any one or more of the features disclosed in U.S. Patent Application Publication No. 2020/0297924 to Daniel Smyth, published on Sep. 24, 2020, U.S. Patent Application Publication No. 2020/0238062 to Kenneth Oplinger, published on Jul. 30, 2020.

And note that embodiments include utilizing any one or more of the devices and/or teachings of the following patent applications and/or any of the patent applications detailed above with respect to implementing the therapeutic actions detailed herein (whether such is controlled per method action 2240 or otherwise simply used to implement a therapy): U.S. Patent Application No. 63/212,582 filed on Jun. 18, 2021, to Wolfram Dueck; U.S. Patent Application No. 63/152,777 filed on Feb. 23, 2021, to Wolfram Dueck, U.S. Patent Application No. U.S. Patent Application No. 63/152,777 filed on Feb. 23, 2021, to Wolfram Dueck, filed on Aug. 20, 2021, to Daniel Smyth, all by way of example only and not by way of limitation. And to be clear, other techniques and devices can be used.

Any device, system, and/or method that can enable the therapeutic treatments detailed herein can be utilized in at least some exemplary embodiments, providing that the art enables such, unless otherwise specified herein.

And also, to be clear, while method 2200 is presented in terms of controlling an implant, whether that be a cochlear implant, or another type of implant separate from the cochlear implant, singularly and/or together, embodiments can include utilizing Sacher any of the exemplary therapeutic substance implementation devices detailed above by reference in combination with any one or more of the method actions herein or otherwise to accomplish the treatment method action detailed herein, providing that the art enables such, unless otherwise noted.

By implementing early postop evaluations to identify early postop changes in the physiology within the cochlea, early intervention can be implemented, which early intervention might not otherwise be implemented in the absence of the teachings detailed herein. As seen above, an early change in impedance within the cochlea, such as an increase as detailed above, at least by a certain amount, within the limited time periods detailed herein, can indicate higher impedance later on, such as two or three or four months thereafter, as noted above. This later impedance rise can be a result of fibrous tissue growth, or cell damage, etc. Accordingly, embodiments include implementing therapies based on a determination that the impedance rise has occurred within one or more of the aforementioned temporal periods detailed above, by one or more of the amounts detailed above. As noted above, comparisons can be made across two or three days, which comparisons can be utilized to predict future impedance scenarios (at least generally, such as statistically higher impedance levels) within the cochlea.

The above noted impedance increases within the above-noted temporal periods can be considered utilitarian predictors of more significant fibrosis to come. In this regard, some fibrosis will occur in almost all cochlear implant scenarios. Embodiments herein forecast the significant fibrosis, as distinguished from normal fibrosis/fibrosis that is expected and/or otherwise that does not result in increased impedances by a significant amount, as contrasted to fibrosis can cause deleterious effects later on, such as an increase in the impedance within the cochlea two or three or four months after implantation. Based on this production, decisions can be made as to whether or not to intervene, where the action of intervening can have by itself deleterious consequences. That said, based on the prediction, a more intense medical therapy can be implemented relative to that which would otherwise be the case, again where the more intense medical therapy could have deleterious effects.

Accordingly, concomitant with the teachings detailed above, in an embodiment, therapy or otherwise the actions taken after the impedance rise is identified, or actions that would not be taken in the absence of some indicator that there is a need for such otherwise that there is an indicator that exists that indicates a statistical possibility that a negative effect will occur at some point in the future. The idea is that the therapies implemented can have, in some instances, statistically significant or at least some heightened non trivial chances of having powerful side effect(s) that would otherwise be unwanted aside from the utilitarian value of utilizing those substances or otherwise these chemical compositions with respect to the concerns identified for providing such. By utilizing the predictive teachings detailed herein, the cost benefit analysis can be better made (or indeed, the cost-benefit analysis can be made in the first instance whereas in some scenarios, a therapist is flying blind with respect to whether or not to implement such therapies, at least prior to the existence of fibrous tissue growth). Accordingly, embodiments include implementing one or more of the therapies detailed herein to reduce or otherwise prevent the growth of fibrous tissue in the absence of any or at least significant fibrous tissue growth or otherwise the absence of the beginnings thereof.

Embodiments include the utilization of predictive biomarkers to forecast the potential for future fibrous tissue growth, and then utilizing these predictive biomarkers to provide preventative treatment that would otherwise not be implemented or otherwise would be less likely to be implemented all other things being equal. As detailed above, embodiments utilize the increase in impedance by the levels detailed herein over the temporal periods detailed herein as a predictive biomarker. It is noted that in some embodiments, anti-fibrotics can be utilized for the treatment, while in other embodiments, and electrode triggered treatment can be utilized. In this regard, electrodes could be utilized at certain frequencies and/or certain currents in a manner that could reduce or otherwise prevent the fibrous tissue growth.

In at least some exemplary scenarios, the increase in the impedance can be a result of an acute inflammatory response, which can be the result of an amount of macrophages which continue to signal for more fibrosis to come into the cochlea. Embodiments thus can utilize the increase in impedance to detect such or otherwise determine that such is the case, and to predict the increase in fibrosis so that a treatment can be implemented. Thus, in an embodiment, within 12, 24, or 36 hours of the cochlear implant electrode array first entering the cochlea, there existed an occurrence in an increase an amount of a substance in the cochlea that causes subsequent fibrous tissue growth, such as, for example, macrophages, and the second set of intra-cochlear measurements captures a latent variable associated with the increase. In an embodiment, it is the increase of the impedance by the various amounts detailed herein with the times detailed herein that corresponds to the latent variable.

Embodiments include the application of systemic antifibrotics (or any other of the treatments detailed herein), such as those that would not be used as a matter of course without justification (as noted above), but the increase in impedance coupled with its predictive value can justify such. In this regard, the risk of giving everyone these drugs as a matter of course without reason is great in some embodiments (and too great in some embodiments). Thus, the methods herein provide for a reasoned based justification for giving a systemic therapy. Moreover, embodiments can include boosting of the baseline treatment based on the increase in impedance. Because of the predictive nature of the increase in impedance, if, after 2 or 3 or 4 or 5 months, there is no impedance rise or otherwise the impedance rise is statistically significantly lower than that which would be expected based on the empirical observations detailed above, the teachings detailed herein can provide an indication that the medication or the therapy was effective.

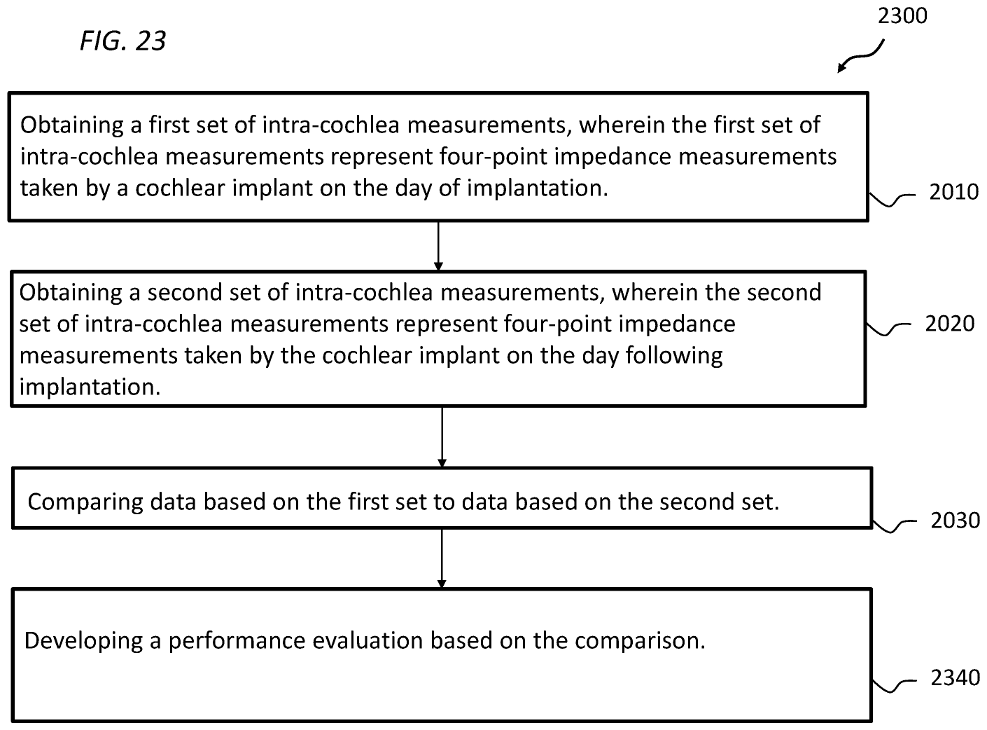

Accordingly, FIG. 23 presents an exemplary flowchart for an exemplary method, method 2300. Method 2300 include method actions 2010, 2020 and, 2030, which actions are described above, and will not be repeated here for purposes of textual economy. Method 2300 further includes method action 2340, which includes the action of developing a performance evaluation based on the comparison. In an embodiment, the performance evaluation is used to evaluate an efficacy of a therapeutic regime implemented based on the comparison. Here, the application of the therapeutic substances have an open-ended scenario in that it is heretofore effectively impossible to know whether the therapy worked per se. Of course, one can look at the absence of fibrous tissue growth with the absence of later increased impedance, or any the other scenarios detailed herein, and declare that the treatment worked. However, it is unclear, typically, as to whether it was the treatment that worked or was it simply a coincidence that the fibrous tissue growth, etc., did not result. Put another way, it is often uncertain whether or not the fibrous tissue growth will actually occur, and thus it is uncertain whether or not the application of the therapy was what prevented the fibrous tissue from growing. But now, with the predictive techniques disclosed herein, there is a much higher level of confidence, approaching if not being certainty, that fibrous tissue growth, or otherwise the higher impedances in the 2 or 3 or 4 or more month time frames, will occur. And thus, if the growth does not occur, or otherwise the high impedances do not occur, after the therapy, there can be a statistically significant otherwise confident correlation between the application of the therapy, and the prevention of the tissue growth or the high impedance. Thus, the teachings detailed herein can provide a performance evaluation that otherwise did not exist, or at least a level thereof that did not exist prior to the teachings detailed herein.

And note that while the method 2300 is presented as its own method, as with any one or more or all of the teachings detailed herein, unless otherwise noted, any one or more of the method actions herein can be combined with any one or more of the other method actions detailed herein. Accordingly, for example, method action 2340 can be combined with method action 2040 detailed above, any method of doing method action 2040 and/or method action 2340 exists in an exemplary embodiment by way of example.

Corollary to the above that in some embodiments, after a given period, such as after two or three months or mor months of providing the therapy, therapy can be stopped. Repeated impedance measurements can be taken during and/or after the therapy is halted. Indeed, the data collections of impedance values at the first and second temporal locations detailed above can be repeated frequently or otherwise as long as the recipient is alive (every day for example, or every hour, or at least every 2 or 3 or 4 or 5 hours or 6 hours or any value or range of values therebetween in one minute increments), and any increase that can be found within the aforementioned time periods can be an indication that future increase in impedance can occur, or that some trauma has occurred or otherwise that the physiological state of the cochlea has changed in a manner that can have deleterious consequences in the future, thus providing a prediction that some deleterious scenario will occur in the future, and the treatment or a new treatment can be implemented accordingly.

Continuing with the concept that the predictive values of the teachings detailed herein can provide an indicator later on that a given therapy was effective, such can provide a basis to stop or otherwise adjust the therapy. Such can also provide a basis to transition from one therapy to another therapy. Based on the evaluation of the performance of therapy, there can then be a basis for a change from the anti-prolific fiber treatment to bone prevention treatment. For example, a non-steroidal anti inflammation can be tried first, and then a clot buster could be tried, and then the therapy could move to anti proliferables, and then to drugs to stop bone formation.

Embodiments include using the teachings herein as a way of early assessment of improvements in new devices. For example, the teachings above can be a biomarker for insertion atraumaticity. That is, a given electrode array of a given model/design has superior insertion characteristics relative to others with respect to reducing/eliminating damage to the cochlea or otherwise causing some form of deleterious event

Accordingly, again with reference to method action 2340, the performance evaluation can be used to evaluate a recurring level of trauma associated with a type of electrode array inserted into the cochlea. By way of example only and not by way of limitation, in an exemplary embodiment, if an impedance rise is seen in the temporal periods detailed herein any certain level in a statistically significant manner for one type of electrode array, such as by a manufacturer or with respect to a given type relative to another type by a same manufacturer, or a given type of electrode array that is an industry standard (for example, pre-curved, straight, banned electrodes, but electrodes, semicircle electrodes, 12 versus 16 versus 22 electrodes on an array, etc.), and evaluation can be made whether or not a given electrode array induces more trauma during insertion relative to other electrode array, all other things being equal. This could provide the basis for altering the use of such arrays and/or provide the basis for developing alternate techniques or otherwise serve as the basis to develop alternat techniques for implanting that array, etc., at least in certain circumstances.

Also, the techniques herein can be used to monitor for repeated bad insertion techniques. Good insertion techniques will have fewer of the impedance increases.

Thus, embodiments include scenarios where the performance evaluation is used to evaluate a reoccurring level of trauma associated with a type of surgical technique used to insert the electrode array in the cochlea. By way of example only and not by way of limitation, this could be used to identify surgeons that implement methods that are less traumatic relative to other surgeons, this could be used to identify implantation techniques that are less traumatic relative to others (e.g., lateral wall hugging vs. perimodiolar hugging or center location, etc.).

Embodiments include automatic measuring. Also, subsequent measurement can be done remotely and/or using an automatic algorithm. But in some embodiments, testing can be done under the control/orders of a person, and can be combined automatic and “manual” testing.

Embodiments of the treatments detailed herein can include anti NBF drugs that prevent new bone formation. Embodiments include using the teachings herein to inform of modifications in surgical techniques, such as using healon flushing or monitoring for surgical improvements.

Note also that in some embodiments, precursor prodrugs can be applied in the middle ear, and based on the increase in impedance, a trigger can be implemented to activate the base drug. Prodrug drugs based on stimulation and/or light and/or radiation in the inner ear and/or the middle ear can be used as part of the therapies. Drug release coatings can be used, and the prodrug can change its composition to make it active and/or inactive, depending on the impedance increase.

In an exemplary embodiment, the therapeutic substances delivered to the recipient owing to the results of the evaluation of the impedance measurement(s) can be so-called “clot-busting” drugs. In an embodiment, the substance delivered can be one or more of Eminase (anistreplase), Retavase (reteplase), Streptase (streptokinase, kibikinase), t-PA, drugs that include Activase, TNKase (tenecteplase), Abbokinase, and/or Kinlytic (rokinase). In an embodiment, the therapeutic substance is a substance to treat thrombolysis.

It is noted that while in many instances, the therapies detailed herein have focused on the application of a therapeutic substance, therapies are not so limited to such. By way of example only and not by way of limitation, therapies that are implemented based on the existence of the increased impedances within the temporal periods detailed herein can include electrical therapy and/or activation therapy as detailed herein. Any therapy that can have utilitarian value with respect to preventing the alternate increase in impedance that is predicted based on the increase in impedance over the day or two after implantation can be utilized in at least some exemplary embodiments. Moreover, other actions can be taken, such as precaution actions or care giver actions, based on the impedance measurements. Some examples associated with electrical therapy and care giver actions will now be detailed.

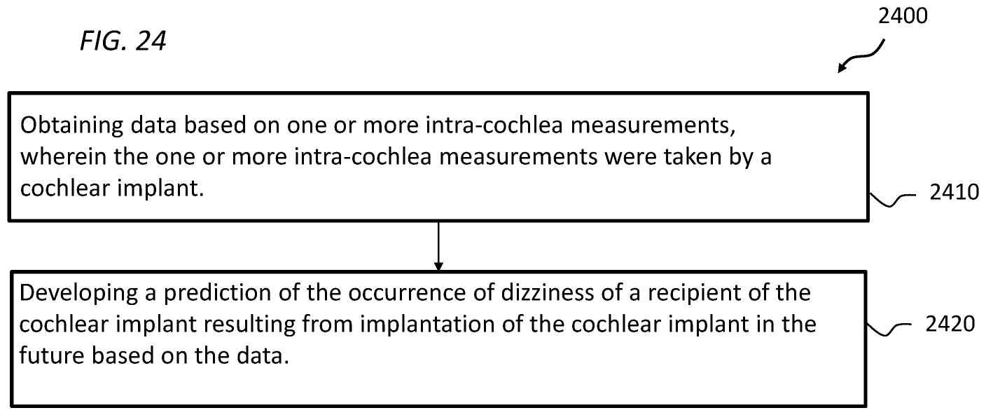

FIG. 24 presents an exemplary flowchart for an exemplary method, method 2400. This method is directed towards addressing issues of dizziness that are associated with implantation of the cochlear implant electrode array. Briefly, method 2400 includes method action 2410, which includes the action of obtaining data based on one or more intra-cochlea measurements, wherein the one or more intra-cochlea measurements were taken by a cochlear implant. Consistent with the teachings above, method action 2410 can be executed by actually utilizing the cochlear implant or otherwise obtaining the data directly there from in real time. By way of example only and not by way of limitation, method action 2400 can further be executed by obtaining the measurements from a third party. In an exemplary embodiment, data is obtained from a remote location, such as a hospital or even a recipient's home (in an exemplary embodiment, the one or more intracochlear measurements of method action 2410 can be obtained in an automatic manner by the implanted cochlear implant, and the measurements can be provided to the external component by way of telemetry, and the external component can provide such to a remote location via a wired or wireless link (for example, the external component can be Bluetooth compatible, and the external component can be in communication with a smart phone or the like, which smart phone can convey the measurements to a remote location—note that some embodiments can have an implant that is Bluetooth compatible)—this embodiment is applicable for the dizziness embodiment or for the other embodiments detailed herein providing that the art enable such). The point is that to execute method action 2410, the actor need not necessarily be operating the cochlear implant or otherwise be obtaining the data directly there from.

In an exemplary embodiment, the one or more intracochlear measurements of method action 2410 are measurements that are taken according to any one or more of the temporal periods detailed herein. In an exemplary embodiment, the measurements are taken within and/or at and/or after 6, 7, 8, 9, 10, 11, 12, 13, 14, 15, 16, 17, 18, 19, 20, 21, 22, 23, 24, 25, 26, 27, 28, 29, 30 him, 31, 32, 33, 34, 35, 36, 37, 38, 39, 40, 41, 42, 43, 44, 45, 46, 47, or 48 hours or more, or any value or range of values therebetween in 0.1 hour increments after the completion of the implantation of the cochlear implant and/or after any of the time triggering actions detailed herein, such as, for example, the cochlear implant electrode array first entering the cochlea. As will be described below, embodiments include taking measurements 72 and/or 96 and/or 120 hours and/or more after the completion of the implementation and/or after any of the time triggering actions detailed herein.

And it is briefly noted that consistent with the teachings detailed herein, the action of obtaining the data can occur after the measurements are taken, and need not be obtained in real time, although embodiments can include such. In an exemplary embodiment, the data is obtained a day or two or three or four or more or any value or range of values therebetween in one hour increments after the measurements are taken.

As can be seen from method 2400, method 2400 does not require taking a second set of measurements at a different temporal location, although it can include such. In this regard, at least some embodiments of method 2400 are directed towards evaluating the impedance values for what they are at the time of acquisition (versus a comparison to how those impedance values may change or have changed at a later time), and predicting a future event based thereon. Accordingly, method 2400 further includes method action 2420, which includes the action of developing a prediction of the occurrence of dizziness of a recipient of the cochlear implant resulting from implantation of the cochlear implant in the future based on the data. Thus, the one or more intra-cochlea measurements obtained at, for example, 24 hours after the completion of implantation, can be evaluated, and based on that evaluation, a determination can be made as to the likelihood that the recipient will experience dizziness resulting from the implantation of the cochlear implant. This is distinguished from, for example, pre-existing dizziness conditions. In this regard, in at least some exemplary scenarios, a recipient will experience dizziness owing to the fact that the electrode array has been placed into his or her inner ear. In other exemplary scenarios, the recipient will not experience dizziness. By evaluating the one or more intracochlear measurements, an estimation as to whether or not the recipient will experience dizziness/a likelihood of such experience, and/or the severity of the dizziness (which can be quantified by a risk scale of a fall, dizziness severity scales, etc., and/or subjective measures of dizziness can be used, and there could be objective concepts such as SCC and otolith function, and any other future way of measuring or evaluating a severity of dizziness), can be made, upon which the aforementioned prediction is based. Briefly, embodiments include a method including executing method 2400, and also obtaining data indicative of whether or not the recipient experienced dizziness prior to implantation of the cochlear implant. The preexisting dizziness could be due to a number of issues/preexisting conditions, and this dizziness could persist after the cochlear implant is implanted. This is discussed in greater detail below. This preexisting dizziness could be a dizziness that the recipient has learned to cope with or otherwise developed coping mechanisms. Moreover, the recipient could be under treatment/have been previously treated, for the underlying disease/ailment and/or the symptoms. Conversely, the dizziness associated with the implantation of the cochlear implant could be a dizziness of severity and/or a type to which the recipient is not accustomed. Thus, there can be utilitarian value with respect to forecasting the dizziness associated with method 2400 irrespective of whether or not the recipient suffers from dizziness associated with other conditions. Accordingly, in an exemplary embodiment, the developed prediction is in addition to any other prediction of dizziness, which other prediction of dizziness is in addition to dizziness resulting from implantation of the cochlear implant. That is, this differentiates from predictions unrelated to the implantation.

In an embodiment, the teachings herein can have utilitarian value with respect to identifying individuals prior to, for example, the individual leaving the hospital/implantation center, who may be “at risk” of future dizziness after the surgery. For instance, if an elderly person becomes dizzy, it may be unsafe for that person to go home (a fall could lead to a broken hip, and a broken hip can often result in an acceleration of end of life scenarios), and thus a recommendation might be made (probably will be made) that the person remains in the hospital (or a mandate, or at least a “refusal” to “permit” discharge—the hospital might not be able to “hold” the person at the hospital, but could refuse to approve discharge), or at least some assisted care center, instead of going home or otherwise not being in some form of care center. Corollary to this is that there could instead be some mandate, or at least recommendation for increased support, such as increased social support, to be put in place. In an embodiment, a visiting nurse or home helper could be assigned or at least recommended. Alternatively, release could be conditional on some 24 hour in-person assistance/other person presence at the residence of the person, or some more limited assistance/other person presence. This can be expensive, or otherwise result in additional costs beyond that which would otherwise be the case (for the extra “housing” (e.g., “use” of a hospital bed/room costs a certain amount per day, with the attendance of hospital personnel, food, checkups, etc. and/or for the extra care at home, and the potential for lost income for the loved one who is in increased attendance with the person, etc.) Accordingly, some way of forecasting/predicting that there might be dizziness resulting from the operation/the implantation of the implant, can be utilitarian, and corollary to this, some form of predicting that there will not be (or will be less likely, in a significant/clinical manner) dizziness resulting from the implant/the surgery, can be utilitarian, as that will impact the discharged time/procedures/conditions. To be clear, embodiments include determining a low likelihood of dizziness owing to the operation/implant, and thus discharging the person under “normal”/non-heightened care/requirements (beyond that which is normal for a cochlear implant recipient of that persons demographic/human factors engineering type, all other things being equal).

And note that in some embodiments, in view of this, the severity of the dizziness (“how dizzy” the person would potentially be for example) is not determined per se, or otherwise is a secondary issue. It is instead the consequence of the dizziness in the context of the individual's health, age and/or independence of living that is the goal of method action 2420 in at least some embodiments. But note that a goal here can be to determine that the forecasted dizziness will be of a different kind or of a different severity than that which the recipient is accustomed to experience. Put another way, if the person has lived with dizziness in the past, the future dizziness could be concomitant with the past dizziness, and thus the recipient may be accustomed there to/understands/knows/has experience in how to protect or otherwise safeguard his or her person against such, or even treat, in a limited manner at least, the dizziness to which he/she has become accustomed. Conversely, the dizziness resulting from the cochlear implant/the surgery, could be of a different kind and/or degree, warranting some extra caution/protection/intervention or otherwise a different course of action.

Thus, embodiments can include prescribing and/or mandating alternate care support, relative to that which would otherwise be the case in the absence of the prediction, for the human based on the prediction. Embodiments can include prescribing and/or mandating an extended stay in a healthcare facility after the surgical procedure based on the prediction relative to that which would otherwise be the case in the absence of the prediction. Embodiments can include prescribing and/or mandating that the human stay at least 24, 36, 48, 60, 72, 84, 96, 120, 144, 168, 192 or more hours or any value or range of values therebetween in 1 hour increments (where prescribing two days corresponds to “at least 48 hours by way of example) in a healthcare facility after the surgical procedure based on the prediction relative to that which would otherwise be the case in the absence of the prediction.

Embodiments include providing/prescribing/mandating, etc., the heightened/alternate/extended care/stay during a period of time where the human is more vulnerable to injury due to dizziness relative to other times. Note that in an embodiment, it can be that the human may leave the healthcare facility where the surgery took place after the surgery (e.g., within 12, 24, 36 or 48 hours of the surgery), and then return or go to another center, at least 24, 36, 48, 60, 72, 84, 96, 120, 144, 168, 192 or more hours or any value or range of values therebetween in 1 hour increments after being discharged/leaving the facility, where during the return period of heightened vulnerability, but upon returning to the facility, or going to the other center, the person has not experienced dizziness, at least not relating to the implant/surgery at that time. In an embodiment, the person does experience dizziness after returning/going to the center.

In an embodiment, elevated basal impedances relative to apical impedances (in the post-operative measurements) can also be used as a predictor. Embodiments thus include comparing one or more impedance values between electrodes or any other impedance values detailed herein associated with the more basal electrodes to one or more impedance values between electrodes or any other impedance values detailed herein associated with the one or more apical electrodes. And consistent with the teachings detailed herein, the mean median and/or mode values of the basal values can be utilized with respect to the data that is to be compared to the apical values, or any of the other statistical data points detailed herein providing that such can provide utilitarian value. And note also that in some embodiments, middle electrodes can be used instead of the apical electrodes and/or in addition to the apical electrodes.

Accordingly, in an exemplary embodiment, method action 2420 results in a prediction that dizziness will occur in the future, and in another exemplary embodiment, method action 2420 results in a prediction that dizziness will not occur in the future. Note that in some embodiments, it can be that the prediction is that it is unclear whether or not dizziness will occur in the future. In an embodiment, the methods herein can indicate when a reliability of a prediction is low or otherwise subject to some further evaluation or question. Dizziness could occur when there is a prediction that it will not occur, and vice versa. In an exemplary embodiment, method 2400 is executed 10, 15, 20, 25, 30, 35, 40, 45 or 50 times for as many different recipients and/or for as many different ears (a recipient could receive two cochlear implants, and thus method action 2400 would be executed for each ear) or any value or range of values therebetween in one increment intervals within a period of time of 3, 4, 5, 6, 7, 8, 9, 10, 11, 12, 13, 14, 15, 16, 17, 18, 19, 20, 21, 22, 23, 24, 25, 26, 27, 28, 29, or 30 weeks or months, or any value or range of values therebetween in one week increments with an accuracy rate of at least 65, 70, 75, 80, 85, 90, 91, 92, 93, 94, or 95%, or any value or range of values therebetween in 1% increments.

In an exemplary embodiment, there are method actions that entail evaluating one or more respective levels of the one or more of the measurements obtained in method action 2410. In an exemplary embodiment, the action of developing the prediction is based on the evaluation of the one or more respective levels. In an exemplary embodiment, the measurements are impedance values, such as any of the impedance values detailed herein. In an exemplary embodiment, the action of evaluating the levels includes determining whether or not the impedance value is at and/or above and/or below a predetermined value. Thus, in an embodiment, the one or more intra-cochlear measurements are one or more impedance measurements, and method 2400 can further include evaluating one or more relative levels of the one or more impedance measurements (e.g., if the level is relatively high or low) and developing the prediction based on the relative level. More on this in a moment.

With respect to the impedance values, a full set of impedance values can be obtained for every electrode pair that is possible (in the case of four (4) point impedance—note that in some embodiments, standard impedance measurements/monopolar impedance (MP1, MP2 or MP1+2) can be used, or otherwise measurements from each electrode to the common return, which can be an extra-cochlear electrode, which can be an electrode contact or ball, or could be an electrode plate on the receiver-stimulator, or a combination of these)). In an embodiment, point-to-point or two-point impedance can be used for impedance measurement can be used such as an intra-cochlear electrode and an extra-cochlear electrode, or an intra-cochlear electrode and another intra-cochlear electrode. That said, in embodiments, the data obtained in method action 2410 includes only a subset of the full set of impedance values that were actually taken. Moreover, the data obtained in method action 2410 can include only a subset of a subset of impedance values that were actually taken relative to the total number of values that could be taken. It is noted that this is the case with any of the embodiments detailed herein. In this regard, in an exemplary embodiment, it could be that impedance values were measured or otherwise taken for every electrode pair, but only the impedance values for the basal and/or apical and/or middle electrodes are obtained in method action 2410. Of course, the full sets of impedance values could be obtained, and only a subset can be utilized with respect to evaluating one or more respective levels of the one or more measurements. By way of example, in an exemplary embodiment, the action of evaluating entails evaluating only the levels for the basal electrodes. Any evaluation of one or more respective levels of the one or more of the measurements that can enable the teachings detailed herein can be utilized in at least some exemplary embodiments.

Note also that embodiments of evaluating one or more respective levels of the one or more of the measurements can include statistical analysis. By way of example only and not by way of limitation, the action of evaluating one or more respective levels can be executed by obtaining a mean, median, and/or mode of the impedance values various electrode pairs (whether the entire set or a subset) and evaluating the level of that mean value for example. More on this in a moment. Thus, the obtained data can be one or more impedance measurements from within the cavity and/or statistically analyzed data of one or more impedance measurements from within the cavity.

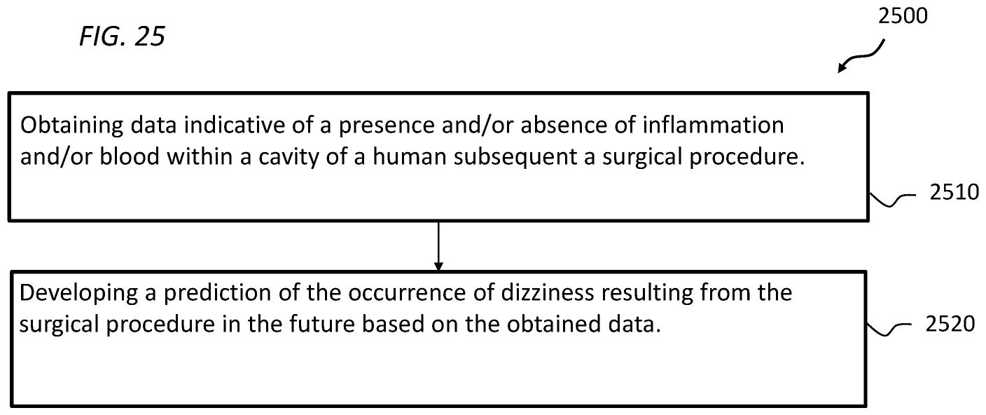

FIG. 25 presents another exemplary flowchart for an exemplary method, method 2500. Method 2500 includes method action 2510, which includes the action of obtaining data indicative of a presence and/or absence of inflammation and/or blood and/or cellularity (number of cells) within a cavity of a human subsequent to a surgical procedure. In an exemplary embodiment, consistent with the teachings herein, this surgical procedure can be the implantation of a cochlear implant electrode array into a cochlea. Still, in other embodiments, this could be the implantation of an electrode or an electrode array into a vestibular duct or some other part of the labryi a human body. Still further, consistent with the teachings herein, impedance values can be utilized to evaluate or otherwise determine whether or not there is the presence and/or absence of inflammation or blood or cellularity within the cavity, such as a cochlea. Accordingly, in an embodiment, the data obtained can be impedance values. That said, in an embodiment, the data can be data based on impedance values. For example, statistical values or harmonized data sets can be the data that is obtained. Note further that in an exemplary embodiment, the data obtained could be imaging data or some other set of information indicative of the presence and/or absence of inflammation and/or blood and/or cellularity within a cavity. Any device, system, and/or method that can enable a determination or otherwise an indication of a presence and/or absence of inflammation and/or blood and/or cellularity within a cavity of a human that can enable the teachings detailed herein can be utilized in at least some exemplary embodiments.

Method 2500 further includes method action 2520, as seen. Method action 2520 entails developing a prediction of a likelihood of occurrence of dizziness of a recipient of the cochlear implant resulting from implantation of the cochlear implant in the future based on the data. In an embodiment, this can be executed by evaluating one or more of the impedance measurements. For example, if the level of impedance is at and/or above a certain value, a determination can be made that there is a likelihood of dizziness that will occur in the future owing to the implantation of the cochlear implant. If the level of the impedance is at and/or below a certain value, a determination can be made that there is a low likelihood of dizziness that will occur in the future owing to the implementation of the cochlear implant.

In an exemplary embodiment, the prediction can be a percentage based prediction. In an exemplary embodiment, the prediction can be that based on the obtained data obtained in method action 2510, there is at least a 35, 40, 45, 50, 55, 60, 65, 70, 75, 80, 85, 90, 95, or 100%, or any value or range of values therebetween in 1% increments, likelihood that the recipient will experience dizziness associated with or otherwise owing to the implantation of the cochlear implant. In an exemplary embodiment, the prediction can be that based on the obtained data obtained in method action 2510, there is no more than a 35, 30, 25, 20, 15, 10, or 5% likelihood that the recipient will experience dizziness associated with or otherwise owing to the implantation of the cochlear implant.

The likelihood level can have utilitarian value with respect to prescribing a treatment or otherwise recommending a course of action in the future. By way of example only and not by way of limitation, if a percentage likelihood of the occurrence of dizziness in the future is above a given percentage, at least relative to a given person, recommendations may be made in order to care for that person. By way of example only and not by way of limitation, if the person, is for example, 70 years old, and thus relatively prone to greater damage to person owing to a fall, it may be recommended that the person utilize a wheelchair for a given period of time during a time period specified after the implantation. In an exemplary embodiment, the recommendation could be that the person not be alone or otherwise be with able-bodied people as much as possible, or otherwise that an able-bodied person spend more time with the recipient for a given period of time. That said, in an embodiment, the person may not necessarily need to be able-bodied. Instead, the person can simply be somebody who can observe or otherwise look out for the recipient and obtain medical attention in the event that dizziness causes a physical problem or otherwise injury to the recipient. Still, in an embodiment, there is utilitarian value with respect to having the recipient comport himself or herself more cautiously with respect to physical activities or otherwise movement. In an exemplary embodiment, the recipient could be recommended to utilize one or more canes and/or crutches or other walking aids for a given period of time, or otherwise at least have one or more readily accessible. For example, the recipient may carry around the cane but may not necessarily use the cane, but if the recipient experiences dizziness, the cane is available and the cane can be used.

In an exemplary embodiment, the recipient could be cautioned against driving a vehicle during a certain period of time and/or cautioned against playing certain sports for a certain period of time, or otherwise recommended to always hold onto a handrail of some sort when moving upstairs or moving around hard surface objects.

Embodiments include prescribing medicaments or otherwise therapeutic substances to treat the dizziness or at least reduce the likelihood of dizziness, alternatively and/or in addition to the above-noted actions (such as extended hospital stay for example). By way of example only and not by way of limitation, any one or more of the therapeutic substances detailed herein can be prescribed. In an embodiment, an anti-emetic is prescribed. In an embodiment, an antihistamine is prescribed. In an embodiment, prochlorperazine is prescribed. In an embodiment, anti-inflammatory substances are provided or at least prescribed. In an embodiment, a higher salt diet or a specific diet is recommended. Antibiotics can be prescribed and/or used. Therapeutic substances to avoid vomiting and/or nausea can be prescribed. In an embodiment, steroid medications can be prescribed. Diuretics can be prescribed. Substances that have combinations of glucocorticoid and mineralocorticoid activity can be prescribed.

In an embodiment, electrotherapy can be utilized to treat the dizziness (which includes avoiding the dizziness). By way of example only and not by way of limitation, the cochlear implant can be utilized to provide a current that can affect the vestibules, or more accurately, affect the signals provided from the vestibules to the brain or otherwise affect the signals provided to the brain to counter or otherwise minimize the effects of the dizziness. In an exemplary embodiment, a vestibular stimulator or an anti-dizziness stimulation device can be applied to the recipient. Indeed, depending on the likelihood of the severity of the dizziness, a second surgery can be undertaken to implant one of these dizziness compensating devices in the recipient. In an embodiment, balance disturbance can be addressed by using electrical stimulation to mask “noise” in the vestibular system. In an embodiment, one or more of the teachings of U.S. Pat. No. 11,351,372 are used in such a method, as well as variations thereof. For example, while stimulus is delivered to the otolith organ in the teachings of the '372 patent, in other embodiments the stimulation is applied to the cochlea using a cochlear implant, which stimulation can be specially controlled by the external component, including using sub-threshold electrical stimulation (or supra-threshold electrical stimulation, if needed to “reach” the vestibular system/nerves thereof).

Thus, embodiments include using electrical stimulation to ameliorate anticipated balance dysfunction following cochlear implant implantation. In one embodiment, there is a method where, based on the prediction of dizziness, there is the action of generating electrical stimulation signals configured to improve the recipient's sense of gravitational balance by masking vestibular noise generated by the peripheral vestibular system, and delivering, via one or more of the plurality of electrodes of the cochlear implant, electrical stimulation signals to the cochlea, which in some embodiments also reach at least the inferior branch of the vestibular nerve. In an embodiment, the cochlear implant includes a stimulator unit configured to generate and deliver one or more continuous electrical pulse trains to the inner ear, which can in some embodiments, reach to the inferior branch of the vestibular nerve of the recipient via one or more of the plurality of electrodes, wherein the one or more continuous electrical pulse trains are configured to suppress, in some embodiments, mostly in the inferior branch of the vestibular nerve and/or in some embodiments, partially in superior branch, erroneous balance information generated by the peripheral vestibular system of the inner ear that would otherwise be sent to the brain of the recipient by the vestibular nerve. Embodiments include methods of doing so/controlling the cochlear implant to do so.

Embodiments include applying one or more continuous pulse trains/delivering such to the inner ear, so that the recipient continually experiences a sense of balance for periods of time, such as throughout the entire day, or at least during a dizzy spell (and thus in an embodiment, the recipient can control the cochlea implant to implement anti-dizziness actions) while the recipient is in an upright position, while performing certain activities, etc. In an embodiment, so as to provide the recipient with an experience of a sense of balance, electrical stimulation signals are generally delivered for extended periods of time to account for the recipient's disbalance in some manner, while taking into account recipient-specific characteristics and the residual effects of the stimulation. In certain examples, the electrical stimulation signals are delivered to the recipient continually constantly through the day (e.g., continually deliver stimulation signals for 8 hours, 12 hours, 14 hours, etc.), and this can start at the forecasted/predicted onset time, or a bit before as a precaution. In other embodiments, the cochlear implant can be controlled to deliver stimulation signals to the inner ear, and ultimately reaching the vestibular nerve in some embodiments, at a specific duty cycle that ensures that the recipient continually experiences a sense of balance, or at least to counteract the dizziness.

In an embodiment, the cochlear implant is used as a “balance prosthesis” or “balance implant.” The implant can be controlled to assist recipients cope with the dizziness. In an embodiment, the implant is an implant according to or a variation of that described in U.S. Pat. No. 9,339,649. In an embodiment, the implant is a combination of a cochlear implant and that of the '649 patent. Embodiments thus can include treating balance disorders in a proactive rather than a reactive manner This can be done by using a vestibular nerve stimulator in some specific embodiments, or at least using the cochlear implant to stimulate, at least subtly, the vestibular nerve, by generating electrical stimulation signals that are specifically configured to evoke a response in one or more segments of the vestibular nerve, such as the vestibular ganglion, inferior branch of the vestibular nerve, and/or the superior branch of the vestibular nerve. That said, in some embodiments, there is stimulation applied to one or more parts of the peripheral vestibular system, such as the ampulla associated with the semicircular ducts.

Thus, embodiments include treating the human with a therapeutic substance and/or an electrostimulation device to reduce the likelihood and/or the magnitude of future dizziness resulting from the surgical procedure relative to that which would otherwise be the case in the absence of the treatment. In an embodiment, the existing implant, such as the one that cause the dizziness in the first instance, is utilized to treat the human, whether through electrostimulation and/or the utilization of a therapeutic substance, which can be delivered from the implant, by way of example. In an embodiment, the cochlear implant can include a vestibular electrode or more than one vestibular electrode, or otherwise can be configured to provide stimulation to the vestibular or the like in a manner that can have utilitarian value with respect to treating dizziness. Embodiments can include utilizing a standard cochlear implant, where a basal electrode or the basal electrodes is/are utilized to provide stimulation pulses that would affect the vestibular nerve.

Still, embodiments can focus on treatment and/or otherwise safeguards and precautions. There is utilitarian value here with respect to forecasting the likelihood so that more serious attempts to mitigate or otherwise safeguard against the deleterious effects of the occurrence of dizziness can be undertaken. In this regard, broad blanket statements directed to the possibility that dizziness can occur without underlying support might be discounted by the recipient or otherwise might not be forcefully explained to the recipient or otherwise the recipient's family if there is no underlying data to support the conclusion that dizziness is likely to occur. By rough analogy, air travelers will often sit without a seatbelt on an airline cruising altitude. But if there is a forecast of turbulence, people are very likely to utilize their seatbelts. This analogy can extend further in that is typically a good idea to always wear a seatbelt in an airplane irrespective of the presence of turbulence. However, people are people, and people do not always engage in best practices or otherwise the most cautious practice. Accordingly, the prediction can be analogous to a prediction of turbulence in the air at a future temporal location, and thus actions can be taken that do not necessarily avoid the turbulence, but mitigate the effects of the turbulence. And so it is that the effects of dizziness can be mitigated by, for example, restraining from engaging in certain activities or certain placements of one's body, and otherwise having a caregiver in closer attendance so that if the dizziness spell occurs, as it is more likely to occur, that person can more readily aid the recipient in his or her moments of need. Embodiments can include “warning” next of kin/loved ones/caregivers, that the recipient may experience dizziness. This is opposed to the recipient having to wait potentially minutes and/or hours for a caregiver to arrive. Indeed, it could be as simple as giving assistance to a recipient to travel from the bathroom to a bedroom, or vice versa. Or to aid in egress from a kitchen, which could have sharp countertops or otherwise hard surfaces to an area with carpeting and/or a sofa.

Embodiments include forecasting a temporal location of an onset of dizziness and/or a length of time of the occurrence of dizziness. By way of example only and not by way of limitation, embodiments can include before 12, 18, 24, 30, 36, 42, 48, 60, 72, 84, 96, 108, 120, 132, or 144 hours, or any value or range of values therebetween in one hour increments after the completion of the implantation of the cochlear implant or otherwise after any of the actions detailed herein that trigger the commencement of the time period, such as first insertion of the electrode array in the cochlea, making or otherwise developing the prediction of the likelihood of occurrence of dizziness, and such onset is likely to occur within 96, 120, 144, 168, 192, 220, 250, or 300 hours or any value or range of values therebetween in one hour increments from the completion of the implantation of the cochlear implant or otherwise after any of the actions detailed herein that trigger the commencement of the time. In an exemplary embodiment, any of the aforementioned percentages of the likelihood of the occurrence of dizziness can be the case for any one or more of these temporal locations. In an exemplary embodiment, the prediction can be that the period of heightened likelihood of dizziness can exist for 5, 6, 7, 8, 9, 10, 15, 20, 25, or 30 days or more, or any value or range of values therebetween in one day increments from the onset of dizziness. Accordingly, embodiments can include providing the aforementioned recommendations of heightened care for those periods of time and otherwise correlating the recommended actions to those time periods and/or temporal locations. A buffer at the beginning and/or end can also be applied. After the period lapses, the recommended course of action would cease to be implemented in at least some exemplary embodiments.

Note also that embodiments can include utilizing actual empirical results. By way of example only and not by way of limitation, if after a certain period of time from the estimated temporal location of the onset of likelihood of dizziness, the recipient has not experienced dizziness or at least severe dizziness for a certain period of time, the therapy or otherwise the recommended actions could be halted. By way of example only and not by way of limitation, if the recipient does not experience dizziness for 48, 60, 72, 96, 120, 144, 168, 192, 220, or 250 hours, or any value or range of values therebetween in one hour increments after a time during the commencement of the believed time period where there is a likelihood of dizziness to occur, the treatment could be stopped. Accordingly, in an exemplary scenario, a prediction can occur that the recipient is likely to experience dizziness owing to the implantation of the cochlear implant commencing at least 168 hours from the completion of the cochlear implantation surgery (it could be before in this scenario, hence the “at least”). If after 96 hours or a week for example from the 168 hour mark the recipient does not experience dizziness, or otherwise does not experience dangerous dizziness or otherwise severe dizziness, the treatment regime or the recommended course of actions can be halted (e.g., a caregiver could return to his or her home or stop living with the recipient, the recipient could begin driving a car again, etc.). This can also be the case if, for example, the recipient experiences dizziness during a certain period of time, and then the recipient has not experienced dizziness or otherwise does not experience severe dizziness for 60 hours for example (or any of the time period noted herein) since his or her last experience of severe dizziness.

Note that these exemplary scenarios are not to be confused with a recipient self reporting that he or she is experiencing dizziness and healthcare provider recommending or otherwise instructing a course of action to address the dizziness. That is not a forecast or a prediction. That is a reaction to an occurrence that has already taken place. Put another way, the forecasts and predictions detailed herein are for occurrences that have not occurred and otherwise would be totally speculative but for the innovative teachings detailed herein. Indeed, by analogy, this could be akin to whether or not an insurance company would sell an insurance policy for dizziness to a recipient of the cochlear implant. If the recipient was experiencing dizziness, an insurance carrier would not sell a policy (at least not at a reasonable rate) to ensure against dizziness because the recipient is already experiencing dizziness. Conversely, an insurance carrier could conceivably utilize the teachings detailed herein to evaluate whether or not an insurance policy should be sold to cover the dizziness or otherwise what premium should be charged. But to be clear, this example is provided so as to give a qualitative example of the utilitarian value of the prediction and to distinguish the prediction from a more pedestrian case of guessing or otherwise after-the-fact analysis.

And note that in an embodiment, the methods detailed herein are executed without taking into account demographic and/or historical features of the recipient. In this regard, in an exemplary embodiment, the controlling factor with respect to method actions 2420 and 2520 and/or to move on to a treatment or otherwise recommended course of action is the data obtained in method actions 2410 and 2510 respectively. Put another way, by way of example only and not by way of limitation, demographic data and/or historical data might or might not be utilized in the evaluation or otherwise as part of the determination process going into developing the prediction or otherwise determining that a recommended course of action or otherwise a treatment regime is appropriate. However, in some embodiments, the controlling factor will be the data obtained in the just noted method actions. In at least some exemplary embodiments, even if the historical data and/or demographic data, etc., indicates that the recipient might be predisposed to dizziness after the implantation, if the data obtained in the just noted method actions indicates a low likelihood of dizziness, that will control, and vice versa.

In an embodiment, the method further includes the action of comparing the obtained data to predetermined data. By way of example only and not by way of limitation, an impedance value obtained for a given electrode (intracochlear electrode) and/or between electrode pairs (intracochlear pairs or at least one intracochlear electrode) can be compared to a predetermined value, where in at least some embodiments, the electrode(s) are all intracochlear electrodes, and in other embodiments, at least one electrode is an intracochlear electrode. Still further by way of example only and not by way of limitation, two or more impedance values can be compared to a predetermined value and/or a plurality of predetermined values. In an exemplary embodiment, the action of developing the prediction is based on whether the comparison shows that the obtained data is higher and/or lower and/or at the predetermined data. In an exemplary embodiment, by way of example only and not by way of limitation, there can be a predefined set of data for various electrode pairs, such as for pairs 2-3, 3-4, 4-5, 5-6 by way of example only and not by way of limitation. In an exemplary embodiment, the predefined set of data can be W, X, Y, and Z impedance values for the respective pairs. If, for example, the impedance values for the pairs are below W, X, and Z respectively, but the value for the pairs 4-5 is higher or at Y by way of example, a prediction can be made that there will be the occurrence of dizziness. That said, in an exemplary embodiment, a determination can be made that there will not be the occurrence of dizziness because there is only one of the four pairs that show the increased value. In an exemplary embodiment, there must be one or two or three or four or five or six or seven or eight or nine or ten or any value or range of values therebetween in one increment that are at and/or above the values for the specific sets for there to be a determination that there will be an increased likelihood of dizziness. Indeed, there can be utilitarian value with respect to “discounting” or otherwise disregarding a limited amount of data as being extraneous. Still further, there can be utilitarian value with respect to taking a statistical analysis of the data, such as taking a mean and/or median and/or mode value of one or more of the impedance values.

In an exemplary embodiment, impedance values for any one or more or all of electrodes 1, 2, 3, 4, 5, 6, 7, 8, 9, 10, 11, 12, 13, 14, 15, 16, 17, 18, 19, 20, 21, or 22, where 1 is the most basal electrode, and/or impedance values for the adjacent electrode pairs with respect to four point impedance can be utilized or otherwise can be obtained and evaluated to make a determination as to whether or not there is an increased likelihood of dizziness. For example, impedance values for the four or five or six most basal electrodes (or pairs of electrodes) can be utilized, and the other impedance values could be disregarded or ignored, if they are obtained in the first instance. That said, impedance values for some electrodes can be weighted more heavily than others. Accordingly, in an exemplary embodiment, impedance values for the four or five or six most apical electrodes (or pairs of electrodes) could be weighted more or less than the impedance values for the other electrodes, such as the aforementioned basal electrodes. And the weighting could be different for each pair or a group of pairs (linear or geometric, for example). The weighting can have utilitarian value with respect to, for example, taking a mean and/or median and/or mode or otherwise taking a statistical analysis of the data.

By way of example only and not by way of limitation, a mean average bulk impedance of all electrode pairs, of the basal-most electrode pairs, of the apical-most electrode pairs, of the middle pairs, of the basal-most pairs and middle pairs, of the middle pairs and apical-most pairs, of the pairs made by electrodes 2-3, 3-4, 4-5, 5-6, 6-7, 7-8, 8-9, 9-10, 10-11, 11-12, 12-13, 13-14, 14-15, 15-16, 16-17, 17-18, 18-19, 19-20, and 20-21 or any combination thereof, including pairs (starting from 2-3) 1 and/or 2 and/or 3 and/or 4 and/or 5 and/or 6 and/or 7 and/or 8 and/or 9 and/or 10 and/or 11 and/or 12 and/or 13 and/or 14 and/or 15 and/or 16 and/or 17 and/or 18 and/or 19 of greater than and/or equal to 130, 135, 140, 145, 150, 155, 160, 165, 170, 175, 180, 185, 190, 195, 200, 205, 210, 215, 220, 225, 230, 235, 240, 245, 250, 260, 270, 280, 290, 300, 310, 320 or 330 22 or any value or range of values therebetween in 0.1 02 increments (e.g., 163.5, 172.4, 166.6, to 186.6, etc.) can be used as a basis to declare an increased likelihood of dizziness, and thus forecast such, in at least some embodiments.

In an embodiment, the data obtained in the method actions detailed above is one or more of (i) a mean four point impedance across at least 3, 4, 5, 6, 7, 8, 9, 10, 11, 12, 13, 14, 15, 16, 17, 18, 19, 20, or 21 electrodes, or any value or range of values therebetween in 1 increment of the cochlear implant (where by way of example, impedance across at least four electrodes could be electrode pairs 2-3 and 3-4 and 4-5, or 2-3 and 5-6, etc.); (ii) maximum four point impedance within a cochlea (e.g., identifying the maximum impedance between two electrodes); (iii) mean impedance within the cochlea (the mean of all the impedance readings); or (iv) maximum impedance at locations within the cochlea corresponding to a basal, middle and/or apical third of the cochlea. In an embodiment, the impedance data is data that is normalized, such as normalized relative to the intra-operative impedances. In an exemplary embodiment, the normalization techniques detailed herein can be utilized. In an embodiment, elevated impedance values relative to the intra-operative impedances by a certain amount can be indicative of future dizziness. Any one or more of these data can be compared to the baseline/threshold and evaluated to make the prediction.

In an embodiment, one or more of the data of the method includes a mean basal four-point impedance value or the method includes developing the mean basal four-point impedance value (e.g., if the data provided is the raw impedance values). And the method can further be such that the developed prediction of the occurrence of dizziness is based on the mean basal four-point impedance value.

Thus, in some embodiments, the data obtained in the method actions is data indicative of impedance between electrodes in at least a basal portion of the cochlea, in at least a middle portion of the cochlea and/or in at least the apical portion of the cochlea. The impedance between could be four point impedance, or could be bulk impedance between electrodes.

Note embodiments can include executing one or more of the actions herein while disregarding surface effects from the electrodes, providing that a given technology implicated enables such, or a variation thereof or an analogous action.

Embodiments include periodically testing/measuring/obtaining impedance values. In an embodiment, the data of the methods detailed above is data taken (as opposed to obtained—the data can be taken and obtained by the same entity, and taken and obtained by different entities, the taken being the actual acquisition using the cochlea implant for example) at at least a first temporal location. This first temporal location can be any of the noted locations above. In an embodiment, the method further includes obtaining second data that is taken at a second temporal location following the first temporal location by at least 10, 15, 20, 25, 30, 35, 40, 45, 50, 55, 60, 65, 70, 75, 80, 85, 90, 95, 100, 110, 120, 130, 140, or 150 hours, or any value or range of values therebetween in 1 hour increments. Note that additional data can be taken in between. Indeed, in an embodiment, the data can be taken at a rate of less than every hour, every two hours or every three hours etc. If subthreshold currents are utilized, under some situations, data can be taken continuously, although in an embodiment, there is utilitarian value with respect to limiting the usage of the cochlear implant until after a healing period has completed. In this exemplary embodiment, the data taken at the first temporal location is consistent with the data taken at the second temporal location. By way of example only and not by way of limitation, the impedance values are above the aforementioned threshold values. This can be the mean median and/or mode variants of evaluating the data or can be the individual data. In an embodiment, the impedance values, or the statistical analyze values, are within 5, 10, 15, 20, 25, 30, 35, or 40% of the data taken at the first temporal location (where that data is utilized as the denominator). In an exemplary embodiment, the method further includes obtaining third data and/or fourth data and/or fifth data and/or six data and/or seventh data and/or eighth data, where the data is taken successively in a manner where the respective temporal locations are separated by at least 10, 15, 20, 25, 30, 35, 40, 45, 50, 55, 60, 65, 70, 75, 80, 85, 90, 95, 100, 110, 120, 130, 140, or 150 hours, or any value or range of values therebetween in 1 hour increments. In an embodiment, the aforementioned phenomena associated with the values is seen for at least some and/or all of these data.

Thus, with respect to embodiments where the method includes identifying a therapy and/or a recommended course of action based on the data, the method can further include continuing the therapy and/or the recommended course of action based on the consistency.

In an embodiment, when data that is taken subsequent the first temporal location is not consistent with the data taken at the first temporal location, this could be an indication in some embodiments that dizziness is no longer likely to occur. In an exemplary embodiment, if the data at the subsequent temporal location meets any of the criteria detailed above with respect to an indication that dizziness is likely not to occur, embodiments can include halting the treatment or otherwise halting the recommended course of actions. Thus, with respect to embodiments where the method includes identifying a therapy and/or a recommended course of action based on the data, the method can further include discontinuing the therapy and/or the recommended course of action based on inconsistency.

Thus, embodiments can include a method where the prediction developed is that there is a likelihood of the occurrence of dizziness of the recipient (including any of the percentage likelihoods detailed above). The method can include recommending a course of action according to any of those herein or other actions. The method further includes the method further includes obtaining second data based on one or more second intra-cochlea measurements, wherein the one or more second intra-cochlea measurements were taken by the cochlear implant at least 72, 96, 120, 144, 168, 192, 220, 250, 270, 300, 325, or 350 hours, or any value or range of values therebetween in 1 hour increments after the one or more intra-cochlea measurements were taken and/or after the completion of the implant or any of the time triggers detailed herein. The method can include the action of developing a second prediction that dizziness is unlikely to occur in the recipient after the temporal location of obtaining the second data. And note that data collection can be taken prior to the temporal period where the second data is taken. Indeed, in an exemplary embodiment, data is collected two or three or four or five or six or seven or eight times or more between the data collection upon which the prediction was based and the second data collection. In an embodiment, data can be taken hundreds or even thousands of times or potentially even tens of thousands of times, providing that such does not deleteriously affect the implantee. In an embodiment, the various method actions detailed herein can be automated. By way of example only and not by way of limitation, the cochlear implant can be programmed to automatically take the impedance measurements, or can be controlled to take such. Indeed, in an embodiment, the analysis of the measurements can be automated as well. An algorithm can exist or otherwise be prepared to determine whether or not there is a likelihood of the occurrence of dizziness. By way of example only and not by way of limitation, if the mean, median, and/or mode of whatever set of electrodes is to be relied upon, or if the raw number(s) meet or exceed a threshold, an automatic determination can be made of the prediction, and/or vice versa (including meeting or being below a threshold). But in common practice, the initial evaluation will be made by a human, and then potentially automated data collection can take place. And thus, in an embodiment, by way of example only and not by way of limitation, the additional data collections can be evaluated or otherwise analyzed and a determination can be made that the elevated impedance levels remain or otherwise that there remains inflammation, and thus the prediction of continued dizziness remains. Conversely, an evaluation can be made automatically to determine that the impedance values are now below or at a threshold indicating that dizziness is not likely in the future. Still, in an embodiment, an evaluation can be made automatically that the values are low or at the threshold, and a human can then make another evaluation to determine (ratify) a prediction that the occurrence of dizziness is not likely. Accordingly, the above-noted method can include developing a second prediction that dizziness is unlikely to occur in the recipient after the temporal location of obtaining the second data. In an embodiment, the outlook for the lack of dizziness can be at least 1, 2, 3, 4, 5, 6, 7, or 8 weeks, and the dizziness is related to the cochlear implant.

Further, in embodiments where remedial action was taken based on the determination of the likelihood of the occurrence of dizziness (e.g., the recommendation that the recipient take a drug, or that the recipient refrain from using ladders, etc.), there are methods where, based on the second prediction, the remedial action is halted (e.g., the recipient is recommended that he or she can do certain things that previously were advised against doing, etc.).

In an embodiment, the dizziness predicted is a dizziness that prevents the recipient from standing straight, without adjusting his or her feet placement, for 5, 10, 15, 20, 25, 30, 35, 40, 45, 50, 55, or 60 seconds. In an embodiment, the dizziness predicted can be a prediction on how long it would take for a person to get out of a chair and/or walk a certain distance and return to the chair. The length of time can be a gauge of dizziness. And the tests herein can be used to estimate the time or otherwise estimate the qualification that would be associated with that time. In an embodiment, the dizziness predicted is a dizziness that prevents the recipient from meeting the just noted baseline immediately after getting up from a seated position. In an embodiment, the predicted dizziness is a dizziness that prevents the recipient from touching his or her nose with eyes closed at least one out of three times within 30, 45 or 50 seconds. In an embodiment, the predicted dizziness is a dizziness that prevents the recipient from passing a manual/physical sobriety test administered pursuant to the laws of the Commonwealth of Virginia, the Commonwealth of Pennsylvania, the United Kingdom, or New South Wales as of Dec. 21, 2022. In an embodiment, the predicted dizziness is a dizziness that prevents the recipient from walking in a straight line with his or her arms extended outward at the side for three (3) meters. Note that in all of these examples, the recipient could do so prior to the onset of dizziness.

In an embodiment, the predicted dizziness is not a debilitating dizziness, but a dizziness that just makes the recipient uncomfortable, irritable, or nauseous. In an embodiment, the predicted dizziness makes the recipient want to lie down.

In an embodiment, without therapeutic substance treatment and/or electrostimulation, the recipient experiences the dizziness at the aforementioned rates.

In an embodiment, dizziness (nystagmus) is measured using rotational chair testing, videonystagmogramphy (VNG), computerized dynamic posturography (CDP) and/or evoked potentials testing. That is, the forecasted/predicted dizziness is a dizziness that would be indicated by one or more of those tests as being dizziness. And in some embodiments, without treatment, the predicted dizziness comes to pass in a manner that would be detected by one or more of those tests. In an embodiment, the dizziness registered by one or more of these tests might or would warrant some form of intervention by the average, reasonable healthcare professional/one of ordinary skill in the art, or otherwise would prompt at least 30, 35, 40, 45, 50, 55, 60, 65, 70, 75, 80, 85 or 90% or more or any value or range of values therebetween in 1% increments of healthcare professionals in the art to recommend intervention.

In an embodiment, the data obtained in method actions 2410 and 2510 is used to assess the severity of the dizziness/forecasting/predicting the severity of the dizziness. In an embodiment, the higher the impedance values (raw or statistics or otherwise), the more severe the dizziness. Thus, embodiments include varying the recommended course of action/treatment, based on the predicted severity. By way of example only and not by way of limitation, if the data obtained in method actions 2410 and/or 2510 indicate that the dizziness will be minor and otherwise not severe, the recommendation could be to simply take some minor precautions or otherwise be on the lookout for the onset of sensation of dizziness and be ready to take actions accordingly. If for example, the prediction is for moderate dizziness, the recommendation might be to avoid driving or otherwise operating heavy machinery or riding a bicycle, etc. If the prediction is for severe dizziness, the recommendation might be to stay in bed and/or stay in a comfortable reclining chair or otherwise a sofa for as much as possible during the forecasted period of time, or otherwise always have another person in attendance or otherwise close by, or otherwise avoid hard objects or sharp objects, such as avoiding the kitchen or the bathroom, and otherwise having someone in attendance while in such areas. Indeed, in an exemplary scenario, a recommendation could be to wear a bicycle helmet or the like when not in bed or when not sitting, as an expedient precaution.

Note that in an embodiment, there is a correlation between the impedance values or the impedance data and the severity with respect to the results of testing for dizziness. By way of example only and not by way of limitation, and impedance value(s) of or greater than 25, 30, 35, 40, 45, or 50% or more of the above-noted impedance values or otherwise threshold values for making a prediction that there is a likelihood of dizziness could indicate a moderate case of dizziness, and way of example only and not by way of limitation, and impedance value(s) of or greater than 35, 40, 45, 50, 55, 60, 65, 70, 80, 90, or 100% or more of the above-noted impedance values or otherwise threshold values for making a prediction that there is a likelihood of dizziness could indicate a severe case of dizziness.

In an embodiment, the recommended courses of action for a prediction of dizziness could be to install handgrips and baths and/or showers and/or near toilets, always use handrails when walking up and down stairs, utilizing a cane and/or walker, practicing exercises that can improve balance, such as vestibular rehabilitation therapy, yoga, tai chi, gymnastics, or taekwondo (post implantation procedures). Recommended courses of action could also be the removal of floor clutter or objects on the floor over which one might trip, or otherwise moving objects that have a high center of gravity, or otherwise corralling small pets and/or children. In some embodiments, fires are not present during a given period of time, and/or the recipient stays away from stoves or the like. Indeed, in an embodiment, hard surfaces, such as a fireplace hearth or the like, can be covered with protective padding. Corollary to this is that in an embodiment, objects that move randomly or even in a pattern might be removed or eliminated or otherwise reduced (in number or occurrence). By way of example only and not by way of limitation, automatic vacuum cleaning robots might be disabled for a period of time. Strobing lights for example will be disabled, and it is possible that televisions will be disabled, or at least the viewing of programming on television will be limited to programs that do not have a lot of movement or chaotic actions. In an exemplary embodiment, a recommendation can be that the recipient sit on the side of the bed for a period of time, such as one or two or three or more minutes, before getting out of bed. In an embodiment, a recommendation can be that the recipient stand up from a sitting position, and then remain standing for 30 seconds or a minute or two or more, before proceeding to walk. In an embodiment, it can be recommended that the recipient avoid [poorly lit and or] dark places or otherwise that the recipient maintain or otherwise keep lights on at a higher level than that which might otherwise be the case. In an exemplary embodiment, the recipient is recommended to avoid high places or to avoid utilizing stairs or ladders, or otherwise wearing, for example, high heels, etc. In an exemplary embodiment, the recipient is recommended against running or swimming or skiing or playing certain sports that involve running or otherwise walking along busy streets or walking on concrete or asphalt surfaces, etc.

In an exemplary embodiment, the methods include retaking the impedance values or otherwise obtaining data indicative of the impedance values or otherwise the information and/or blood in the cochlea. These actions can be executed 5, 6, 7, 8, 9, 10, 11, 12, 13, 14, 15, 20, 25, 30, 35, 40, 45, 50, 55, 60, 70, 80, or 90 days, or more after the completion of the implantation surgery and/or any of the triggers detailed herein. In an exemplary embodiment, the data can be reevaluated, and if the data indicates a low likelihood of future dizziness owing to the cochlear implant implantation, the recommendations can be voided or otherwise changed. Conversely, if the data indicates continued likelihood of dizziness, the recommendations can be maintained or otherwise modified. Indeed, if the data indicates increased severity, more drastic measures or recommendations can be taken or otherwise at least recommended. Accordingly, embodiments can include executing any one or more of the methods detailed above, and making a recommendation that the recipients take any one or more of the actions detailed herein owing to the determination that there is a high likelihood of the recipient experiencing dizziness in the future owing to the cochlear implant. Then, after for example, five or 10 or 15 days, or any of the time periods detailed above, data can be retaken and then the data can be obtained and analyzed, and depending on the results of the data, further recommendations can be made and/or prior recommendations can be rescinded.

It is briefly noted that embodiments include discounting dizziness that occurs within the first, second, third, and/or fourth days after implantation. In an exemplary embodiment, the recommendations are based primarily or at least the controlling information with respect to whether or not to recommend actions to take a precaution owing to the prediction of future dizziness are based on the data obtained from inside the cochlea or otherwise the cavity and/or the data relating to the inflammation and/or blood in the cochlea or otherwise the cavity by way of example. Indeed, in an exemplary embodiment, there exists the method of warning the recipient or otherwise providing the recipient recommendations to take within the first week after implantation, and then indicating to the recipient that the recipient need not worry about dizziness owing to the cochlear implant after five or six or seven or eight or nine or 10 or 11 or 12 days from implantation or whatever time trigger is applicable.

Thus, embodiments include forecasting/predicting post-cochlear implantation dizziness. Dizziness can include unsteadiness, lightheadedness, new-onset vertigo and/or non-specific dizziness. In an embodiment, the dizziness is independent of age, cause for hearing loss and preoperative vestibular function. In an embodiment, the recipient does not have preexisting dizziness/is not afflicted with a condition that causes preexisting dizziness, and does not have preexisting dizziness associated with their original cause for hearing loss (e.g., Meniere's disease, delayed endolymphatic hydrops, autoimmune inner ear disease).

Embodiments include using, to measure or otherwise ascertain inflammation, such as a degree of inflammation, a level of inflammation, as quantified by impedance for example, four-point impedance (4PI). Embodiments include using 4PI to measure the bulk impedance between electrodes, and in an embodiment, an increased density of cells between measuring electrodes (e.g., from blood, inflammatory cells or fibrosis) would result in higher impedance. In embodiments, there is a positive correlation between 4PI and percentage of intracochlear fibrosis following cochlear implantation as well as the presence of blood.

In an embodiment, the predictions are not associated with a prediction of dizziness within one or two or three days of completion of implantation of the cochlear implant. In this regard, the experience of dizziness at the one or two day mark can be, in some embodiments, more prevalent, and thus forecasting this may be akin to forecasting that there will be precipitation next month.

In an embodiment, the information used to determine whether or not there is an increased likelihood dizziness is the mean basal 4PI (calculated from the mean of the basal-most 6 measurements). In an embodiment, mean 4PI is used and/or maximum basal 4PI is used, and the values of such or compared to a predetermined value to determine whether or not there is an increased likelihood of dizziness. Thus, in an embodiment, if the value is below a predetermined value and/or equal to a predetermined value, a determination is made that there is not an increased likelihood of dizziness, and vice versa.

Embodiments can include, but are not limited to, utilizing the basal-most 1 or 2 or 3 or 4 or 5 or 6 or 7 or 8 measurements and not using other measurements to make the determination as to whether there will be an increased likelihood of dizziness.

In an embodiment, post-operative CI-related dizziness at 1-week are predicted in recipients that have statistically higher 1-day mean basal 4PI, and, in some embodiments, this significant difference in mean basal 4PI continues at 1-week. In an embodiment, the values of the impedance (mean median and/or mode and/or individual impedance value(s) for example) can be at least 50, 75, 100, 125, 150, 175, 200, 225, 250, 275, 300, 350, or 400 percent, or any value or range of values therebetween in 1% increments for those recipients who are predicted to have an increased likelihood of dizziness more than that which would be for recipients who are not predicted to have an increased likelihood of dizziness. In an exemplary embodiment, the patients who are predicted to have an increased likelihood of dizziness have those impedance values relative to recipients that do not show or otherwise ultimately do not show symptoms of dizziness in the time frames detailed herein. In an exemplary embodiment, the patients who are predicted to have an increased likelihood of dizziness have those impedance values relative to at least 50, 55, 60, 65, 70, 75, 80, 85, 90, 95, or 100% or any value or range of values therebetween in 1% increments of the recipients who do not experience dizziness during the aft aforementioned time periods over at least 10, 15, 20, 25, 30, 35, 40, 45, 50, 55, 60, 65, 70, 75, or 80 consecutive predictions.

In an embodiment, the predicted dizziness and/or the actual resulting dizziness is a postoperative cochlear implant related dizziness, characterized by new unsteadiness (unsteadiness that did not exist prior to the implantation) and/or the absence of rotational vertigo, occurring at one week after the completion of the surgery.

In an embodiment, at 1 week after the completion of the surgery, there is no correlation between mean basal 4PI and SVV angle.

In an embodiment, the inflammation identified is utricular inflammation. In other embodiments, the inflammation is different from utricular inflammation. In an embodiment, the recipients that ultimately do experience dizziness do not experience acute labyrinthine dysfunction.

Techniques herein aim to preserve the patient's pre-operative natural hearing in order to achieve electro-acoustic stimulation, allowing for an improved hearing experience relative to that which would otherwise be the case. Although “soft” surgical techniques have been extensively developed, patients are still losing residual hearing post cochlear implantation in more than half the cases. There are generally two types of hearing loss reported: immediate or delayed hearing loss. Immediate hearing loss may be a consequence of intra-cochlear injury during implantation where integral structures within the cochlea are damaged, resulting in cochlear cell death or a disruption in the mechanics involved in natural hearing pathways. Delayed hearing loss may occur several months following cochlear implantation with the determining factors largely unknown. A foreign body reaction and fibrosis formation can often provoke a delayed loss of hearing. At least some embodiments include using readings from within the cochlea to avoid or otherwise reduce the likelihood of these occurrences relative to that which would otherwise be the case in the absence of the teachings herein.

Electrical impedance, which is a measure of the electrical opposition a circuit presents when current is applied, can be used to obtain data regarding an environment within a cochlea of a human. These can be used to assess the implant's integrity and compliance for stimulation. Impedance can be measured in two ways (at least). First is the use of a monopolar or common ground configuration, where common ground impedance (CGI) stimulates one intra-cochlear electrode while the remaining electrodes are used as the ground, and is largely influenced by the changes occurring at the electrode-tissue interface.

A second impedance measurement, four-point impedance (4PI), can be used. As detailed above, 4PI involves four adjacent intra-cochlear electrodes, with the current applied to the two outer electrodes while the voltage differential is measured between the inner two. This configuration minimizes the effect of the electrode-tissue interface as different electrodes are used for stimulation and voltage recording, which allows for a more direct measurement of the environment surrounding the electrode array. Four-point impedance was an outcome metric in a clinical trial testing a steroid-eluting electrode array, an array believed to reduce inflammation within the cochlea, with the results showing decreased values in the steroid eluting group compared to control. The research on the use of 4PI is mixed. One clinical report involving 2 participants reported no significant differences in 4PIs from 1-day to 1-year post cochlear implantation.

There is currently no measure that can be utilized as an early detection for inflammation and fibrosis as it relates to hearing outcomes. Conversely, the tests mentioned above identified fluctuations in impedance measurements following cochlear implantation, and identified relationships to cochlear pathologies that could potentially affect hearing. Specifically, changes in four-point impedance measure were tracked over time, and this provided insight into changes in the intracochlear environment after implantation. The above noted testing investigated cochlear implant recipients who received the Cochlear 532/632 perimodiolar array™ which can be used for patients with minimal residual hearing.

In the above-noted testing, electrical impedance was measured in adult cochlear implant candidates. All candidates were implanted with a Cochlear CI532 or CI632 Nucleus implant™, a perimodiolar array with 22-half band electrodes and a pre-curved design (Cochlear Ltd., Sydney, Australia). Insertions were made into the scala tympani either via an extended round window approach or cochleostomy anteriorly to the round window. Implant switch-on occurred 2 weeks post-implantation. Candidates had no measurable hearing (or >80 dB HL) from an audiometry prior to implantation and therefore no hearing outcomes were collected. The surgical approach (either cochleostomy or round window insertion) was recorded.

Impedance measurements were taken immediately after the implant was placed in its final position during surgery; 1 day-, 1 week-, 4/6 weeks- and 3 months-post-implantation. Stimulation was a charge-balanced biphasic pulse, with a duration of 25 μs and an inter-phase gap of 7.5 μs at 120 current levels (approx. 0.11 mA). A single voltage measurement was taken at the end of the first phase of the biphasic pulse, using the inbuilt voltage measurement function of the implant. The measurements were transferred to an external sound processor, connected to a Freedom™ programming device (POD) interfaced by USB with a PC laptop (Dell, TX, U.S.A.). Two types of impedances were measured in the testing: CGI and 4PI. Common ground produces one measurement per intra-cochlear electrode, thus producing 22 per stimulus. However, for 4PI, since the implant contains 22 intra-cochlear electrodes, it provides 19 sets of 4 consecutive electrodes along the array, thus 19 impedance measurements.

High-resolution temporal bone CT scans were obtained post cochlear implantation to assess angular depth of insertion and other occurrences that affect impedances such as tip fold-over. To do this, the raw DICOM files were analyzed through multi-planar reconstruction (MPR) using HOROS medical image viewer (The Horos Project, Geneva, Switzerland). A single layer image with a thickness of 150 μm, displaying an optimal view of the cochlea, including all intra-cochlear electrodes, round window and the modiolus of the cochlea, was used for analysis. Adapting from a technique previously described by Miguel et al. (Miguel A'R, Argudo A A, Barreiro S A B, González J C F, Macías A R. Imaging evaluation of electrode placement and effect on electrode discrimination on different cochlear implant electrode arrays. European Archives of Oto-Rhino-Laryngology. 2018; 275(6):1385-1394.), coordinates were collected for all intra-cochlear electrodes, round window and the modiolus of the cochlea which were then analyzed in a custom-made script in MATLAB (Mathworks) to obtain the angular insertion depth.

All statistical analyses were performed using R Statistical Software (version 4.04 (R Core Team. R: A Language and Environment for Statistical Computing. R Foundation for Statistical Computing Vienna, Austria 2021.), packages: emmeans (Lenth R V. emmeans: Estimated Marginal Means, aka Least-Squares Means 2021. R package version 1.6.1.), lme4 (Bates D, Mächler M. Bolker B, Walker S. Fitting Linear Mixed-Effects Models Using lme4. Journal of Statistical Software. 2015; 67(1):1-48)). Linear mixed-effects models were fitted to the impedance data. In all models, the impedance data was transformed on a logarithmic scale in order to ensure model assumptions were met, however results are reported after reverse-transform of the data. The first linear mixed-effects model was fitted to the impedance per electrode to compare impedances along the electrode array across time and between impedance types. Electrode number and time along with all interaction terms were included as fixed effects. Electrode number was treated as a categorical variable. Random effects were patient, time within patient, and electrode number within patient. This was applied to both CGI and 4PI data. A second model was fitted to determine different patterns in 4PI amongst patients after cochlear implantation. Changes in the average 4PI measurements across time were normalized to the average impedance measured intra-operatively. The model resembled the previous model; however, the patients were separated into groups depending on the changes seen at 1-day post-surgery. This model was fitted to CGI and 4PI. No adjustments for multiple comparisons were made.

From 21 cochlear implant recipients, 88 4PI measurements were collected with 21 measurements intra-operatively, 19 1-day, 16 at 1-week, 17 at 4/6 weeks and 15 at 3-months. For CG, 84 measurements were obtained, 18 from surgery, 19 at 1-day, 15 at 1-week, 17 at 4/6 weeks and 15 at 3-months.

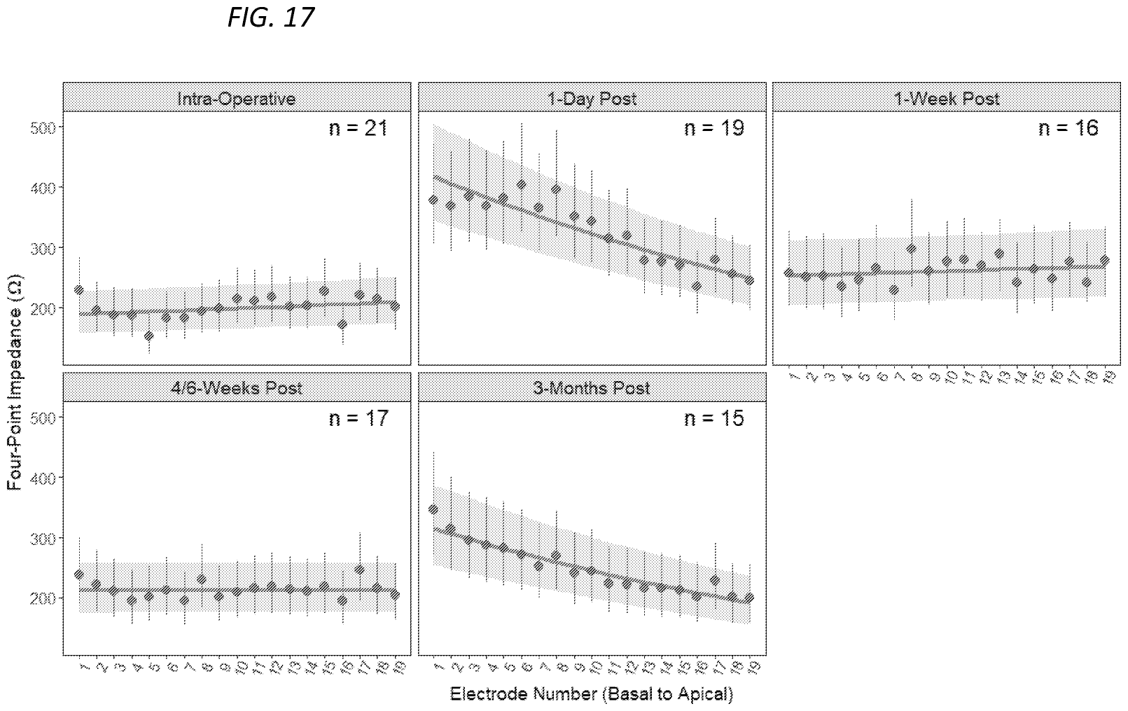

Estimated marginal means were calculated for every electrode along the array at across time for CGI and 4PIs. Overall tests from the first linear mixed model indicated time, electrode number, and the interaction between the electrode number and time effect 4PI (All p<0.002). FIG. 17 displays the means for all 19 4PI measurements at each time point, with 1 being the most basal measurement and 19 the most apical. Intra-operatively, the impedance was similar along the electrode array with an average of 205 Ω and a maximum impedance of 229 Ω at the most basal measurement (measurement 1). Comparing intra-operative to 1-day, impedances increased for all electrodes with a mean impedance of 342 Ω. The highest values occurred at the 8 most basal measurements with an average of 340 Ω. The impedances reduced from measurement 9 to 13, ranging from 311 Ω to 248 Ω, while the 6 most apical measurements had an average of 232 Ω. The increase in impedance from intra-operative to 1-day post was, on average, 138 Ω for measurements 1 to 10 (All p<0.05). At 1-week after implantation, the basal measurements were lower than at 1-day, and were of similar magnitude to the remaining electrodes along the array. There was an increase in impedance along the array from intra-operative to 1-week, with an average increase of 70 Ω. Impedances at 4/6 weeks decreased to levels similar to intra-operative with a slightly higher average of 220 Ω. At 3-months, impedances increased across the array with a decreasing trend from basal to apical. From electrode 1 to 14, the impedance decreased from a maximum of 350 Ω to 220 Ω. The remaining electrodes had similar magnitudes with an average of 211 Ω, a slight increase from intra-operative with the remaining electrodes maintaining this average. The 8 most basal measurements had, on average, an 85 Ω increase from intra-operative to 3 months (All p<0.025).

Linear trends were calculated for 4PI at all time points, with electrode number treated as a continuous variable. FIG. 17 displays the linear trends and 95% confidence intervals along the array (grey area). Note the trends were fitted while impedance data was transformed on a logarithmic scale, hence some curvature in the lines. Intra-operative, 1-week and 4-6 weeks exhibited similar trends, being relatively straight with a slight increase from basal to apical. There were no significant differences in trends between intra-operative with 1-week (mean=0.0006, p=0.850), intra-operative with 4-6 weeks (mean=0.003, p=0.218) and between 1-week and 4-6 weeks (mean=0.003, p=0.350). The linear trends resembled each other at 1-day post and 3-months post, featuring elevated basal measurements and decreasing apically (mean=−0.006, p=0.717).

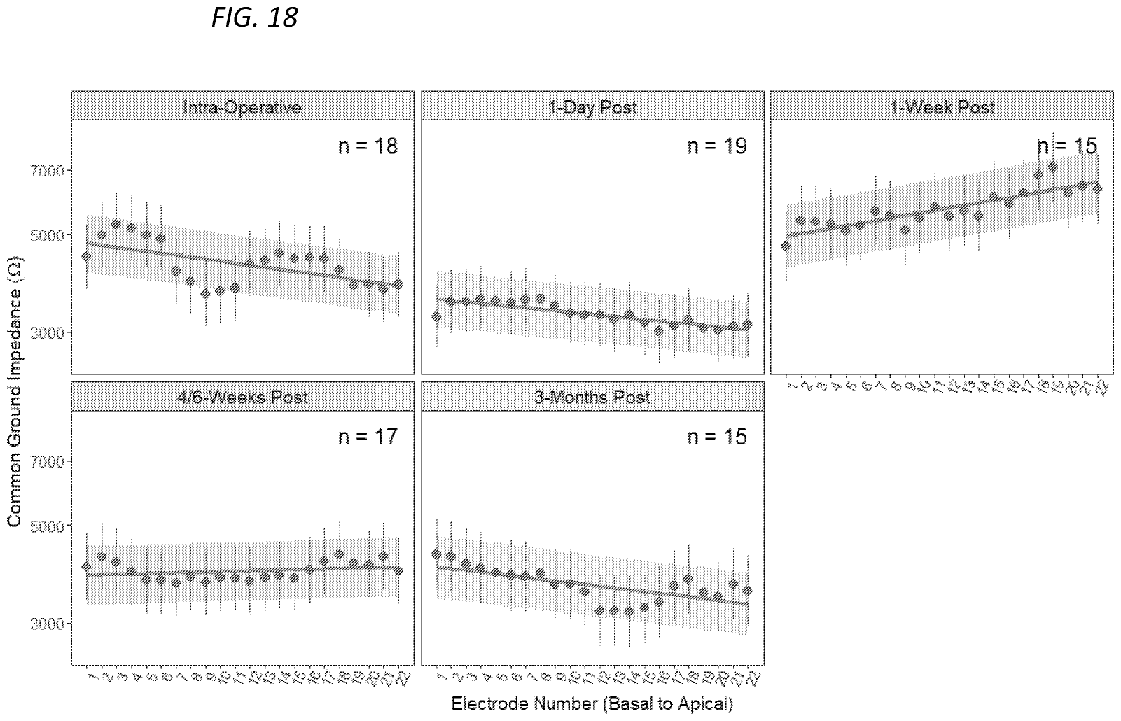

For CGI, the estimated marginal means can be seen in FIG. 18. Overall tests showed electrode number, time and the interaction between the two effected CGI measurements (All p<0.009). Intra-operatively, impedance fluctuated across the array, with an average of 4278 Ω. At 1-day, all impedances were lower than the intra-operative levels, with the average decreasing by 986 Ω (p=0.043). At 1-week, the impedance exhibited an increasing trend from basal to apical, with a minimum value of 4693 Ω for the most basal electrode and a maximum of 7119 Ω at electrode 19. Electrodes 7 to 22, excluding 12, 13, 14 and 16, were significantly different from all other time points (All p<0.045). At 4/6 weeks and 3-months, the measurements decreased to levels marginally lower than intra-operative with an average decrease of 349 Ω (p=0.898) and 626 Ω (p=0.497), respectively.

Linear trends were quantified for CGI at all time points (FIG. 18, grey area). The linear trends were fitted whilst the impedance data was transformed on a logarithmic scale. Intra-operative, 1-day and 3-months displayed decreasing trends from basal to apical. These trends were not significantly different from each other (All p>0.114). Linear trends at 1-week and 4-6 weeks increased from basal to apical however were significantly different (mean=0.011, p<0.001).

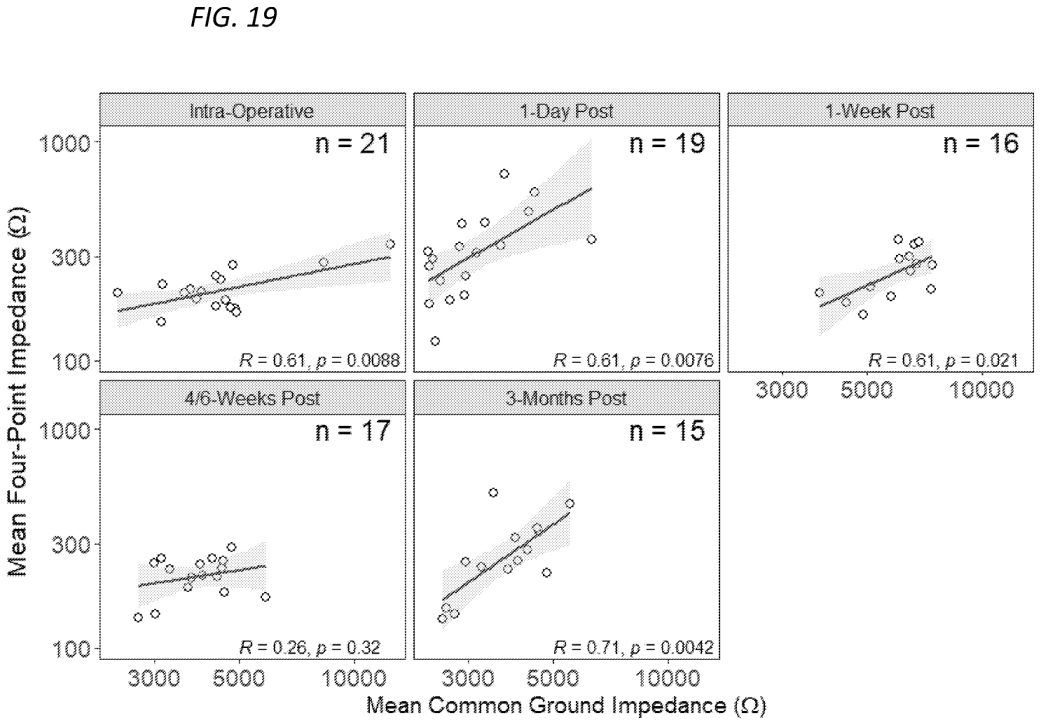

The average common ground and 4PI was calculated for every patient at all time points. FIG. 19 displays the comparison between the two impedance measurements for all time points, including linear regression lines fitted to the data. Strong correlations were found between four-point and CGI at intra-operative, 1-day, 1-week and 3-months. Further analysis was conducted to assess whether the increases seen in 4PI at 1-day post were correlated to the increases in CGI at 1-week post. The mean impedances were calculated for 4PI at 1-day post and CGI 1-week post for every patient. A Pearson's correlation was performed and showed there was no relationship regarding these two time points and impedance measurements (r=0.031, p=0.92, Pearson's correlation).

Further analysis was performed to assess whether larger increases in 4PI at 1-day were correlated with increases at other time points. The average 4PI was calculated for all time points and normalized to the intra-operative average for every patient. Patients were subdivided into two groups; those who experienced less than a 10% increase (n=5) in 4PI at 1-day compared to intra-operative, and those who had an increase higher than 10% (n=10). The 10% cut-off was based on visual inspection of the data. All normalized changes were averaged across all time points and plotted with their respective 95% confidence intervals for both groups (FIG. 4). Overall tests indicated 4PI did not significantly vary across time points for the low group (p=0.491), but did for the high group (p<0.001). At 1-day, the normalized changes from intra-operative were on average 1.265 higher for the high group when compared to the low group (p<0.001), with the low group exhibiting a slight decrease (mean=0.973). Minimal differences were shown at 1- and 4/6 weeks post, however the normalized change at 3-months was, on average, 0.710 higher than the low group (p=0.012). Interestingly, the difference from intra-operative levels at 1-day post and 3-months post were strongly correlated for electrodes 3 to 12 (r>0.560).

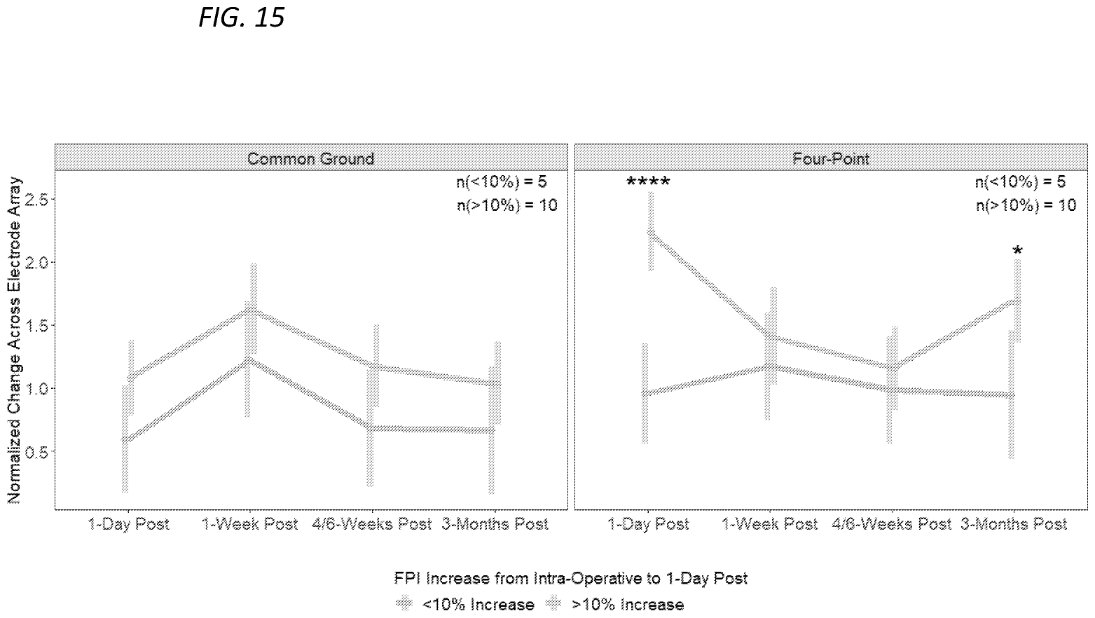

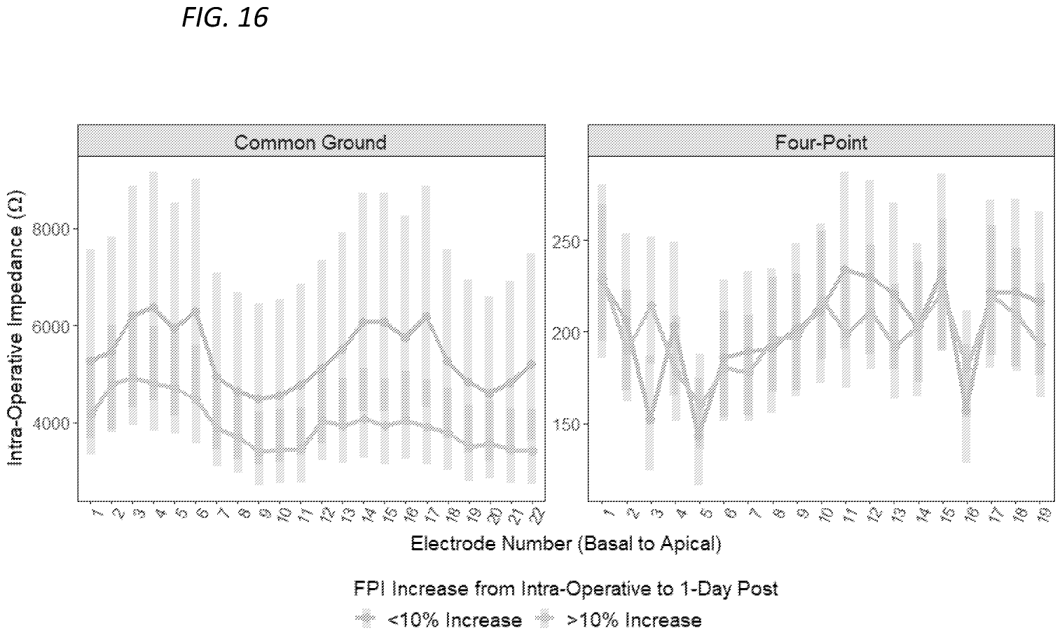

The same analysis was performed on CGI, maintaining the same subgroups as the 4PI data (FIG. 15). Overall tests showed the high group had significant changes across time (p=0.035) while the low group did not (p=0.102). No significant differences between subgroups were detected, however the high group maintained higher normalized changes for all time points. Four-point and common ground impedances were compared between the two subgroups at intra-operative (FIG. 16). The 4PI across the electrode array was almost identical between groups with no significant differences. For CGI, the low group displayed higher impedance measurements throughout the array when compared to the high group but was not significant.

Temporal bone CT scans were collected for 12 patients, 6 from each group. Results showed the average angular insertion depths to be similar across both groups (<10% mean=390°,>10% mean=412°). The difference was not significant (p=0.260). Furthermore, surgical approach did not influence angular insertion depth nor 4PI fluctuations post-implantation as the number of patients that had cochleostomies or extended round windows were equal in both groups.

It was found that the two impedance measurements investigated in the study associated with the above testing are influenced by different aspects of the environment surrounding the electrode array. Common ground impedance is heavily affected by the electrode-tissue interface, representing cell adherence to the electrode surface. However this was found to have minimal effect on 4PI, which instead closely represents the environment in the perilymphatic space in close proximity to the stimulated electrodes (16, 17, 20, 22, 23, 31).

Four-point impedance at 1-day substantially increased, particularly in the basal region of the cochlea.

In this study, increases in 4PI 1-day post implantation were observed in a subgroup of patients. There was no sign these increases are a direct consequence of physical trauma considering intra-operative impedances were similar across both groups (FIG. 16). However these increases may indicate the extent of the acute inflammatory response initiated by cell death pathways. Since impedance measure the electrical opposition of the applied current, an influx of inflammatory cells into the perilymphatic space would influence measurements. The impedance of a conductive fluid, such as perilymph, is relatively low as it presents little opposition to the current, however if cells infiltrated the perilymphatic space the applied current is unable to penetrate through the cells' membranes. This inability is believed to reduce the volume available to conduct current, resulting in elevated impedance measurements.

Further mechanical trauma may have transpired during insertion due to surgical approach. An extended round window or cochleostomy were used in the insertion of the CI532 perimodiolar array in this study. Both approaches require drilling of the cochlea wall in order to reach the scala tympani which may cause bleeding, the infiltration of bone dust into the cochlea, or a loss of perilymph from the scala tympani. All events mentioned above encourage a more extensive inflammatory response and may have contributed to the increased impedances at the basal region.

It is believed that the increases in 4PI were not caused by changes occurring at the electrode-tissue interface, but rather a change within the environment of the scala tympani (because CGI did not increase within 24 hours). Increases presented at 1-week in CGI align is believed to be a result of electrode polarization due to a build-up of cells on the electrode surface causing the impedance to increase, which is reduced when the implant is turned on. When the implant is inactive, cells are able to consistently adhere to the electrode surface, increasing impedance measurements. Once the implant is switched on 2 weeks after implantation, electrical stimulation commences and subsequently blows the cells off the electrode surface. As a result, CGI decreases and remains relatively stable thereafter. Electrode polarization did not affect 4PI or had a negligible impact, supporting the observation that changes at the electrode-tissue interface have minimal effect on 4PI measurements.

The 4PI at 3-months increased in the basal region of the cochlea, similar to 1-day post-implantation. A chronic inflammatory response is anticipated to initiate in the months following cochlear implantation. This may be accompanied by fibrous tissue formation as part of the natural healing process, or in conjunction with chronic inflammation, in addition to areolar tissue and new bone growth. The increase in the basal region implies fibrosis formation is situated in this region. This is concomitant with the formation of fibrous tissue originating from the insertion site and decreasing in volume as it progresses throughout the cochlea.

Statistical analysis revealed 4PI at 1-day was highly predictive of 3-months impedance in cochlear implant recipients. This indicates that the severity of the acute inflammatory response in the cochlea may be correlated with the extent of the chronic inflammatory response, and subsequent fibrosis formation. Contrary to the strong correlations between common ground and four-point impedance (FIG. 19), CGI did not have the same prediction power as 4PI, and did not increase at the time points aligned with inflammatory responses. The subgroup with changes larger than 10% in 4PI did present higher CGIs at all time points. This may be indicative of more cell adherence to the electrode surface or greater fibrosis formation surrounding the electrode array. It is believed that the increases seen in 4PI related to an extensive inflammatory response.

At least some exemplary embodiments according to the teachings detailed herein utilize advanced learning signal processing techniques, which are able to be trained or otherwise are trained to detect higher order, and/or non-linear statistical properties of signals. An exemplary signal processing technique is the so called deep neural network (DNN). At least some exemplary embodiments utilize a DNN (or any other advanced learning signal processing technique) to process a signal representative of captured sound, which processed signal is utilized to evoke a hearing percept. At least some exemplary embodiments entail training signal processing algorithms to process signals indicative of captured light or the reflected sonic energy and/or radio frequency energy. That is, some exemplary methods utilize learning algorithms or regimes or systems such as DNNs or any other system that can have utilitarian value where that would otherwise enable the teachings detailed herein to analyze the data captured by the electrodes. Some embodiments use an expert system.

A “neural network” is a specific type of machine learning system, and embodiments include using a neural network to analyze the data captured by the sensor. Any disclosure herein of the species “neural network” constitutes a disclosure of the genus of a “machine learning system.” While embodiments herein focus on the species of a neural network, it is noted that other embodiments can utilize other species of machine learning systems accordingly, any disclosure herein of a neural network constitutes a disclosure of any other species of machine learning system that can enable the teachings detailed herein and variations thereof. To be clear, at least some embodiments according to the teachings detailed herein are embodiments that have the ability to learn without being explicitly programmed. Accordingly, with respect to some embodiments, any disclosure herein of a device or system constitutes a disclosure of a device and/or system that has the ability to learn without being explicitly programmed, and any disclosure of a method, at least one that constitutes analysis, constitutes actions that results in learning without being explicitly programmed for such.

Some of the specifics of the DNN utilized in some embodiments will be described below, including some exemplary processes to train such DNN. First, however, some of the exemplary methods of utilizing such a DNN (or any other system that can have utilitarian value) will be described.

It is noted that in at least some exemplary embodiments, the DNN or the product from machine learning, etc., is utilized to achieve a given functionality and/or method action as detailed herein. In some instances, for purposes of linguistic economy, there will be disclosure of a device and/or a system that executes an action or the like, and in some instances structure that results in that action or enables the action to be executed. Any method action detailed herein or any functionality detailed herein or any structure that has functionality as disclosed herein corresponds to a disclosure in an alternate embodiment of a DNN or product from machine learning, etc., that when used, results in that functionality, unless otherwise noted or unless the art does not enable such.

Accordingly, embodiments can use a DNN or a product from machine learning or other types of artificial intelligence systems to analyze the data based on data from the electrodes (which could be the data directly from the sensors, or data compiled based on the data from the sensors).

In an exemplary embodiment, there is a product of machine learning of analyzing the various data herein, and that product is a chip that is fabricated based on the results of machine learning. In an exemplary embodiment, the product is a neural network, such as a deep neural network (DNN). The product can be based on or be from a neural network. In an exemplary embodiment, the product is code. In an exemplary embodiment, the product is a logic circuit that is fabricated based on the results of machine learning. The product can be an ASIC (e.g., an artificial intelligence ASIC). The product can be implemented directly on a silicon structure or the like. Any device, system and/or method that can enable the results of artificial intelligence to be utilized in accordance with the teachings detailed herein, such as in a hearing prosthesis or a component that is in communication with a hearing prosthesis, can be utilized in at least some exemplary embodiments. Indeed, as will be detailed below, in at least some exemplary embodiments, the teachings detailed herein utilize knowledge/information from an artificial intelligence system or otherwise from a machine learning system.

Exemplary embodiments include utilizing a trained neural network to implement or otherwise execute at least one or more of the method actions detailed herein, and thus embodiments include a trained neural network configured to do so. Exemplary embodiments also utilize the knowledge of a trained neural network/the information obtained from the implementation of a trained neural network to implement or otherwise execute at least one or more of the method actions detailed herein, and accordingly, embodiments include devices, systems and/or methods that are configured to utilize such knowledge. In some embodiments, these devices can be processors and/or chips that are configured utilizing the knowledge. In some embodiments, the devices and systems herein include devices that include knowledge imprinted or otherwise taught to a neural network. The teachings detailed herein include utilizing machine learning methodologies and the like to establish sensory prosthetic devices or supplemental components utilized with sensory prostatic devices (e.g., a smart phone), implement at least some of the teachings herein. By way of example, additional testing can be executed, and the results of the testing can be provided to a neural network for analysis, and the product of that can be used to ascertain whether or not to implement a treatment regime.

In exemplary embodiment, there can be a component that can be a processor or a chip “programmed” or having access to programming to execute one or more of the functions herein.

And to be clear, in an exemplary embodiment, there are products of machine learning algorithms (e.g., the code from the trained machine learning algorithm) that are included in any one or more of the systems/subsystems detailed herein, that can be utilized to analyze any of the data obtained or otherwise available disclosed above that can be utilized or otherwise is utilized to evaluate the utilitarian value of any one or more of the implants detailed herein. This can be embodied in software code and/or in computer chip(s) that are included in the system(s).

An exemplary system includes an exemplary device/devices that can enable the teachings detailed herein, which in at least some embodiments can utilize automation. That is, an exemplary embodiment includes executing one or more or all of the methods detailed herein and variations thereof, at least in part, in an automated or semiautomated manner using any of the teachings herein. Conversely, embodiments include devices and/or systems and/or methods where automation is specifically prohibited, either by lack of enablement of an automated feature or the complete absence of such capability in the first instance.

It is further noted that any disclosure of a device and/or system detailed herein also corresponds to a disclosure of otherwise providing that device and/or system and/or utilizing that device and/or system.

It is also noted that any disclosure herein of any process of manufacturing other providing a device corresponds to a disclosure of a device and/or system that results there from. Is also noted that any disclosure herein of any device and/or system corresponds to a disclosure of a method of producing or otherwise providing or otherwise making such.

An exemplary system includes an exemplary device/devices that can enable the teachings detailed herein, which in at least some embodiments can utilize automation, as will now be described in the context of an automated system. That is, an exemplary embodiment includes executing one or more or all of the methods detailed herein and variations thereof, at least in part, in an automated or semiautomated manner using any of the teachings herein.

It is further noted that any disclosure of a device and/or system detailed herein also corresponds to a disclosure of otherwise providing that device and/or system and/or utilizing that device and/or system.

It is also noted that any disclosure herein of any process of manufacturing other providing a device corresponds to a disclosure of a device and/or system that results there from. Is also noted that any disclosure herein of any device and/or system corresponds to a disclosure of a method of producing or otherwise providing or otherwise making such.

Any embodiment or any feature disclosed herein can be combined with any one or more or other embodiments and/or other features disclosed herein, unless explicitly indicated and/or unless the art does not enable such. Any embodiment or any feature disclosed herein can be explicitly excluded from use with any one or more other embodiments and/or other features disclosed herein, unless explicitly indicated that such is combined and/or unless the art does not enable such exclusion.

Any function or method action detailed herein corresponds to a disclosure of doing so an automated or semi-automated manner.

While various embodiments of the present invention have been described above, it should be understood that they have been presented by way of example only, and not limitation. It will be apparent to persons skilled in the relevant art that various changes in form and detail can be made therein without departing from the spirit and scope of the invention.

1-24. (canceled)

25. A method, comprising:
detecting a near-term physiological change resulting from implantation of a cochlear implant electrode array in a cochlea and/or detecting a near-term latent variable change that is impacted by a physiological change resulting from the implantation of the cochlear implant electrode array using the cochlear implanted electrode array;
evaluating a percentage change of the detected near-term physiological change and/or the detected near-term latent variable change; and
controlling a component of the cochlear implant and/or another implanted component implanted in the human based on the evaluation.

26. The method of claim 25, further comprising:
controlling the component of the cochlear implant based on the evaluation, wherein the action of controlling the cochlear implant executes an implementation of a therapeutic substance delivery regime.

27. The method of claim 25, further comprising:
controlling the component of the cochlear implant based on the evaluation, wherein the action of controlling the cochlear implant releases a therapeutic substance from the cochlear implanted in the human at the time that the cochlear implant was implanted.

28. (canceled)

29. The method of claim 25, further comprising:
controlling the component of the cochlear implant based on the evaluation, wherein the action of controlling the cochlear implant controls a mechanical component of the cochlear implant to release a chemical from the cochlear implant, which chemical would not be released in a short-term period in the absence of an evaluation of the percentage change being above or at a certain level.

30. (canceled)

31. The method of claim 25, wherein:
one of the detected changes is an increase in impedance within a duct of the cochlea in which the electrode array is located.

32. (canceled)

33. The method of claim 25, wherein:
the action of evaluating the percentage change includes comparing first data obtained during the surgery or effectively immediately thereafter to second data obtained after 18 hours after the first data was obtained.

34-35. (canceled)

36. The method of claim 25, wherein:
the percentage change of the near-term change detected is at least a 10% increase in a mean value of four-point impedance values for at least 15 measurement locations along the electrode array.

37-68. (canceled)

69. A method comprising:
obtaining data based on one or more intra-cochlea measurements, wherein the one or more intra-cochlea measurements were taken by a cochlear implant; and
developing a prediction of a likelihood of occurrence of dizziness of a recipient of the cochlear implant resulting from implantation of the cochlear implant in the future based on the data.

70. The method of claim 69, wherein:
the prediction is that dizziness will occur in the future.

71. (canceled)

72. The method of claim 69, further comprising:
evaluating one or more respective levels of the one or more of the measurements, wherein
the action of developing the prediction is based on the evaluation of the one or more respective levels.

73. The method of claim 69, wherein:
the one or more intra-cochlear measurements is one or more impedance measurements.

74. (canceled)

75. The method of claim 69, wherein:
the one or more intra-cochlear measurements are one or more impedance measurements; and
the method further comprises evaluating one or more relative levels of the one or more impedance measurements and developing the prediction based on the relative level.

76. The method of claim 69, wherein the data is one or more of:
a mean four point impedance across at least 8 electrodes of the cochlear implant;
maximum four point impedance within a cochlea;
mean impedance within the cochlea; or
maximum impedance at locations within the cochlea corresponding to a basal, middle and/or apical third of the cochlea.

77. The method of claim 69, further comprising:
obtaining data indicative of whether or not the recipient experienced dizziness prior to implantation of the cochlear implant, wherein
the developed prediction is in addition to any prediction of dizziness that is in addition to dizziness resulting from implantation of the cochlear implant.

78-80. (canceled)

81. A method comprising:
obtaining data indicative of a presence and/or absence of inflammation and/or blood within a cavity of a human subsequent a surgical procedure; and
developing a prediction of the occurrence of dizziness resulting from the surgical procedure in the future based on the obtained data.

82. The method of claim 81, wherein:
the surgical procedure is implantation of a cochlear implant electrode array implanted in a cochlea.

83. The method of claim 81, wherein:
the data obtained is indicative of a presence of inflammation and/or blood within a cochlea of a human; and
the prediction of the occurrence of dizziness is that dizziness will occur within 10 days.

84. The method of claim 81, wherein:
the data obtained is data indicative of impedance between electrodes within a cochlea;
the impedance is indicative of inflammation and/or blood and/or cellularity within the cochlea, and thus the data is indicative of the presence of inflammation and/or blood within the human; and
the impedance between electrodes within the cochlea is obtained within 36 hours of the completion of the surgical procedure.

85-88. (canceled)

89. The method of claim 81, wherein:
the data is data taken at least a first temporal location;
the method further includes obtaining second data that is taken at a second temporal location following the first temporal location by at least 120 hours;
the data taken at the first temporal location is consistent with the data taken at the second temporal location.

90. The method of claim 89, further comprising:
identifying a therapy and/or a recommended course of action based on the data; and
continuing the therapy and/or the recommended course of action based on the consistency.

91-94. (canceled)
